(19) United States
(12) Patent Application Publication
(10) Pub. No.: US 2024/0166731 A1
(43) Pub. Date: May 23, 2024

(54) HUMANIZED ANTIBODIES AGAINST PAIRED HELICAL FILAMENT TAU AND USES THEREOF

(71) Applicant: Janssen Biotech, Inc., Horsham, PA (US)

(72) Inventors: Rupesh Nanjunda, Horsham, PA (US); Partha Chowdhury, Horsham, PA (US); Fern Sha, Horsham, PA (US)

(21) Appl. No.: 18/284,209

(22) PCT Filed: Mar. 25, 2022
(86) PCT No.: PCT/IB2022/052763

§ 371 Date: Sep. 26, 2023

Related U.S. Application Data
(60) Provisional application No. 63/269,225, filed on Mar. 11, 2022.
(60) Provisional application No. 63/166,524, filed on Mar. 26, 2021.

(51) Int. Cl. C07K 16/18; C07K 16/28; C12N 15/63

(52) U.S. Cl. CPC C07K 16/18; C07K 16/2881; C07K 16/2896; C12N 15/63; C07K 2317/24; C07K 2317/34; C07K 2317/565; C07K 2317/622; C07K 2317/92; C07K 2319/00

(57) ABSTRACT

Humanized anti-PHF-tau antibodies and antigen-binding fragments thereof are described. Also described are nucleic acids encoding the antibodies, conjugates and fusion constructs of the antibodies, compositions comprising the antibodies, methods of producing the antibodies and using the antibodies for treating or preventing conditions such as tauopathies.

### FIELD OF THE INVENTION

The application relates to humanized anti-PHF-tau antibodies, antibody conjugates, nucleic acids and expression vectors encoding the antibodies, recombinant cells containing the vectors, and compositions comprising the antibodies. Methods of making the antibodies, methods of using the antibodies to treat conditions including tauopathies, and methods of using the antibodies to diagnose diseases such as tauopathies are also provided.

### REFERENCE TO SEQUENCE LISTING SUBMITTED ELECTRONICALLY

This application contains a sequence listing, which is submitted electronically via EFS-Web as an ASCII formatted sequence listing with a file name “065768.98US2 Sequence Listing” and a creation date of Mar. 8, 2022, and having a size of 125 kb. The sequence listing submitted via EFS-Web is part of the specification and is herein incorporated by reference in its entirety.

### BACKGROUND OF THE INVENTION

Alzheimer's Disease (AD) is a degenerative brain disorder characterized clinically by progressive loss of memory, cognition, reasoning, judgment and emotional stability that gradually leads to profound mental deterioration and ultimately death. AD is a very common cause of progressive mental failure (dementia) in aged humans and is believed to represent the fourth most common medical cause of death in the United States. AD has been observed in ethnic groups worldwide and presents a major present and future public health problem.

The brains of individuals with AD exhibit characteristic lesions termed senile (or amyloid) plaques, amyloid angiopathy (amyloid deposits in blood vessels) and neurofibrillary tangles. Large numbers of these lesions, particularly amyloid plaques and neurofibrillary tangles of paired helical filaments, are generally found in several areas of the human brain important for memory and cognitive function in patients with AD.

The current AD treatment landscape includes only therapies approved to treat cognitive symptoms in patients with dementia. There are no approved therapies that modify or slow the progression of AD. Potential disease modifiers are anti-amyloid antibodies including Eli Lilly's Donanemab, a humanized IgG1 monoclonal antibody recognizing Aβ(p3-42), a pyroglutamate form of Aβ and aducanumab, a human IgG1 monoclonal antibody against a conformational epitope found on AD. These therapies, and most other potential disease modifiers that may launch in the next decade, target Aβ (the principle component of the amyloid plaques that are one of the two “hallmark” pathological signs of AD).

Neurofibrillary tangles, the second hallmark pathological sign of AD, are primarily composed of aggregates of hyper-phosphorylated tau protein. The main physiological function of tau is microtubule polymerization and stabilization. The binding of tau to microtubules takes place by ionic interactions between positive charges in the microtubule binding region of tau and negative charges on the microtubule lattice (Butner and Kirschner, J Cell Biol. 115(3):717-30, 1991). Tau protein contains 85 possible phosphorylation sites, and phosphorylation at many of these sites interferes with the primary function of tau. Tau that is bound to the axonal microtubule lattice is in a hypo-phosphorylation state, while aggregated tau in AD is hyper-phosphorylated, providing unique epitopes that are distinct from the physiologically active pool of tau.

A tauopathy transmission and spreading hypothesis has been described and is based on the Braak stages of tauopathy progression in the human brain and tauopathy spreading after tau aggregate injections in preclinical tau models (Frost et al., J Biol Chem. 284:12845-52, 2009; Clavaguera et al., Nat Cell Biol. 11:909-13, 2009).

Developing therapeutics preventing or clearing tau aggregation has been of interest for many years and candidate drugs, including anti-aggregation compounds and kinase inhibitors, have entered in clinical testing (Brunden et al., Nat Rev Drug Discov. 8:783-93, 2009). Multiple studies have been published that show the beneficial therapeutic effects of both active and passive tau immunization in transgenic mouse models (Chai et al., J Biol Chem. 286:34457-67, 2011; Boutajangout et al., J Neurochem. 118:658-67, 2011; Boutajangout et al., J Neurosci. 30:16559-66, 2010; Asuni et al., J Neurosci. 27:9115-29, 2007). Activity has been reported with both phospho-directed and non-phospho-directed antibodies (Schroeder et al., J Neuroimmune Pharmacol. 11(1):9-25, 2016).

Despite the progress, there remains a need for effective therapeutics that prevent tau aggregation and tauopathy progression to treat tauopathies such as AD, and other neurodegenerative diseases.

### BRIEF SUMMARY OF THE INVENTION

The application satisfies this need by providing humanized anti-PHF-tau antibodies or antigen-binding fragments thereof that have high binding affinity towards paired helical filament (PHF)-tau and are selective for phosphorylated tau. Humanized antibodies of the application were generated by human framework adaptation (HFA) of mouse PHF-tau-specific antibodies. It is thought that the selectivity of the antibodies for phosphorylated tau allows for efficacy against pathogenic tau without interfering with normal tau function. The application also provides nucleic acids encoding the antibodies, compositions comprising the antibodies, and methods of making and using the antibodies. Humanized anti-PHF-tau antibodies or antigen-binding fragments thereof of the application inhibit tau seeds, as measured by cellular assays using tau seeds derived from HEK cell lysates or from spinal cord lysates from mutant tau transgenic mice. In addition, a chimeric antibody with variable regions of anti-PHF-tau antibodies of the application and mouse Ig constant regions, such as mouse IgG2a constant regions, blocked seeding activity in an in vivo mutant tau transgenic mouse model.

The progression of tauopathy in an AD brain follows distinct special spreading patterns. It has been shown in preclinical models that extracellular phospho-tau seeds can induce tauopathy in neurons (Clavaguera et al., PNAS 110(23):9535-40, 2013). It is therefore believed that tauopathy can spread in a prion-like fashion from one brain region to the next. This spreading process would involve an externalization of tau seeds that can be taken up by nearby neurons and induce further tauopathy. While not wishing to be bound by theory, it is thought that anti-PHF-tau antibodies or antigen-binding fragments thereof of the application prevent tau aggregation or the spreading of tauopathy in the brain by interacting with phospho-tau seeds.

In one general aspect, the application relates to an isolated humanized antibody or an antigen-binding fragment thereof that binds PHF-tau.

In another general aspect, the application relates to an isolated humanized antibody or antigen-binding fragment thereof that binds to a tau protein at an epitope of the tau protein consisting of or within the amino acid sequence of SEQ ID NO: 1, wherein the antibody or antigen-binding fragment thereof binds paired helical filament (PHF)-tau, preferably human PHF-tau.

According to a particular aspect, the application relates to an isolated humanized antibody or antigen-binding fragment thereof that binds to a tau protein at an epitope of the tau protein consisting of or within the amino acid sequence of SEQ ID NO: 1, wherein the antibody or antigen-binding fragment thereof binds paired helical filament (PHF)-tau, preferably human PHF-tau, wherein the epitope of the tau protein comprises one or more of phosphorylated T427, phosphorylated S433 and phosphorylated S435 of the tau protein, but does not comprise all of phosphorylated T427, phosphorylated S433 and phosphorylated S435.

According to another particular aspect, the isolated humanized antibody or antigen-binding fragment thereof comprises immunoglobulin heavy chain complementarity determining regions (HCDRs) HCDR1, HCDR2 and HCDR3 having the polypeptide sequences of SEQ ID NOs: 4, 5 and 6, respectively; and immunoglobulin light chain complementarity determining regions (LCDRs) LCDR1, LCDR2 and LCDR3 having the polypeptide sequences of SEQ ID NOs: 7 or 14, 8 and 9, respectively; wherein the isolated humanized antibody or antigen-binding fragment thereof comprises a heavy chain variable region having a polypeptide sequence at least 90% identical to SEQ ID NO: 12 or 18, and a light chain variable region having a polypeptide sequence at least 90% identical to SEQ ID NO: 13, 19, 23 or 59.

According to another particular aspect, the isolated humanized antibody or antigen-binding fragment thereof comprises a heavy chain variable region having the polypeptide sequence of SEQ ID NO: 12 or 18, and a light chain variable region having the polypeptide sequence of SEQ ID NO: 13, 19, 23 or 59.

According to another particular aspect, the isolated humanized antibody or antigen-binding fragment thereof comprises:
(a) a heavy chain variable region having the polypeptide sequence of SEQ ID NO: 12, and a light chain variable region having the polypeptide sequence of SEQ ID NO: 13;
(b) a heavy chain variable region having the polypeptide sequence of SEQ ID NO: 18, and a light chain variable region having the polypeptide sequence of SEQ ID NO: 19;
(c) a heavy chain variable region having the polypeptide sequence of SEQ ID NO: 12, and a light chain variable region having the polypeptide sequence of SEQ ID NO: 23; or
(d) a heavy chain variable region having the polypeptide sequence of SEQ ID NO: 18, and a light chain variable region having the polypeptide sequence of SEQ ID NO: 59.

According to another particular aspect, the isolated humanized antibody or antigen-binding fragment thereof comprises:
(a) a first heavy chain having the polypeptide sequence of SEQ ID NO: 15 or 20;
(b) two light chains each independently having the polypeptide sequence of SEQ ID NO: 16, 21, 24 or 60; and
(c) a second heavy chain having the polypeptide sequence of SEQ ID NO: 17 or 22.

According to another particular aspect, the isolated humanized antibody or antigen-binding fragment thereof comprises:
(a) a heavy chain having the polypeptide sequence of SEQ ID NO: 61 or 62; and
(a) a light chain having the polypeptide sequence of SEQ ID NO: 16 or 24, or 21 or 60, respectively.

In another general aspect, the application relates to a conjugate comprising an isolated humanized antibody or antigen-binding fragment thereof of the application coupled to an anti-CD98 or anti-TfR antibody or antigen-binding fragment thereof.

According to a particular aspect, the anti-CD98 or anti-TfR antibody or antigen-binding fragment thereof binds to a CD98, preferably human CD98hc, or a TfR, preferably human TfR1, respectively, with a dissociation constant K_{D} of at least 1 nM, preferably 1-500 nM, at neutral pH and an off-rate constant k_{d} of at least 10⁻⁴ sec⁻¹, preferably 10⁻⁴ to 10⁻¹ sec⁻¹, at an acidic pH, preferably pH 5.

According to another particular aspect, the anti-CD98 or anti-TfR antibody or antigen-binding fragment thereof has an off-rate constant k_{d} of 2×10⁻² to 2×10⁻⁴ sec⁻¹, preferably 8×10⁻³ sec⁻¹ at the neutral pH.

According to another particular aspect, the anti-CD98 or anti-TfR antibody or antigen-binding fragment thereof comprises a heavy chain variable region comprising HCDR1, HCDR2 and HCDR3, and a light chain variable region comprising LCDR1, LCDR2 and LCDR3, wherein:
(a) the HCDR1, HCDR2, HCDR3, LCDR1, LCDR2 and LCDR3 of the anti-CD98 antibody or antigen-binding fragment thereof have the amino acid sequences of SEQ ID NOs: 26, 27, 28 or 33, 29, 30 and 31, respectively; or
(b) the HCDR1, HCDR2, HCDR3, LCDR1, LCDR2 and LCDR3 of the anti-TfR antibody or antigen-binding fragment thereof have the amino acid sequences of SEQ ID NOs: 35, 36, 37, 38, 39 and 40, respectively.

According to another particular aspect, the anti-CD98 or anti-TfR antibody or antigen-binding fragment thereof is a single-chain variable fragment (scFv) comprising the heavy chain variable region covalently linked to the light chain variable region via a linker, preferably, the linker has the amino acid sequence of SEQ ID NO: 50 or 51, more preferably, the scFv comprises an amino acid sequence having at least 80%, such as at least 85%, 90%, 95% or 100%, sequence identity to the amino acid sequence of SEQ ID NO: 25, SEQ ID NO: 32, SEQ ID NO: 34, or SEQ ID NO: 41.

In another general aspect, the application relates to a fusion construct comprising a conjugate of the application, wherein the anti-CD98 or anti-TfR antibody or antigen-binding fragment thereof is covalently linked to the carboxy terminus of only one of the two heavy chains of the isolated humanized antibody or antigen-binding fragment thereof via a linker, preferably wherein the linker has the amino acid sequence of SEQ ID NO: 52.

According to a particular aspect, each of the two heavy chains of the isolated humanized antibody or antigen binding fragment thereof comprises one or more heterodimeric mutations, such as a modified heterodimeric CH3 domain, or one or more knob and hole mutations, as compared to a wild-type CH3 domain polypeptide.

According to another particular aspect, the modified heterodimeric CH3 domain of the first heavy chain comprises amino acid modifications at positions T350, L351, F405, and Y407, and the modified heterodimeric CH3 domain of the second heavy chain comprises amino acid modifications at positions T350, T366, K392 and T394, wherein the amino acid modification at position T350 is T350V, T350I, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V or T366M, the amino acid modification at position K392 is K392F, K392L or K392M, and the amino acid modification at position T394 is T394W, and wherein the numbering of amino acid residues is according to the EU index as set forth in Kabat.

According to another particular aspect, the modified heterodimeric CH3 domain of the first heavy chain comprises mutation T366W, and the modified heterodimeric CH3 domain of the second heavy chain comprises mutations T366S, L368A and Y407V.

According to another particular aspect, the isolated humanized antibody or antigen-binding fragment thereof comprises one or more mutations in the Fc domain that enhance binding of the fusion to the neonatal Fc receptor (RcRn), preferably the one or more mutations enhance the binding at an acidic pH, more preferably the Fc has the M252Y/S254T/T256E (YTE) mutations, wherein the numbering of amino acid residues is according to the EU index as set forth in Kabat.

According to another particular aspect, the isolated humanized antibody or antigen binding fragment thereof comprises one or more mutations in the Fc domain that reduce or eliminate the effector function, preferably the Fc has one or more amino acid modifications at positions L234, L235, D270, N297, E318, K320, K322, P331, and P329, such as one, two or three mutations of L234A, L235A and P331S, wherein the numbering of amino acid residues is according to the EU index as set forth in Kabat.

In another general aspect, the application relates to a fusion construct comprising:
(a) a first heavy chain having an amino acid sequence selected from the group consisting of SEQ ID NOs: 42-49;
(b) two light chains each independently having an amino acid sequence selected from the group consisting of SEQ ID NOs: 16, 21, 24 and 60; and
(c) a second heavy chain having an amino acid sequence selected from the group consisting of SEQ ID NOs: 17 and 22.

In another general aspect, the application relates to an isolated nucleic acid encoding an isolated humanized antibody or antigen-binding fragment thereof of the application, a conjugate of the application, or a fusion construct of the application.

In another general aspect, the application relates to a vector comprising an isolated nucleic acid of the application.

In another general aspect, the application relates to a host cell comprising an isolated nucleic acid of the application or a vector of the application.

In another general aspect, the application relates to a method of producing a humanized antibody or antigen-binding fragment thereof of the application, a conjugate of the application, or a fusion construct of the application, comprising culturing a cell comprising a nucleic acid encoding the humanized antibody or antigen-binding fragment thereof, the conjugate, or the fusion construct under conditions to produce the humanized antibody or antigen-binding fragment thereof, the conjugate, or the fusion construct, and recovering the humanized antibody or antigen-binding fragment thereof, the conjugate, or the fusion construct from the cell or cell culture.

In another general aspect, the application relates to a pharmaceutical composition comprising an isolated humanized antibody or antigen-binding fragment thereof of the application, a conjugate of the application, or a fusion construct of the application, and a pharmaceutically acceptable carrier.

In another general aspect, the application relates to a method of blocking tau seeding in a subject in need thereof, comprising administering to the subject a pharmaceutical composition of the application.

In another general aspect, the application relates to a method of inducing antibody dependent phagocytosis (ADP) without stimulating secretion of a pro-inflammatory cytokine in a subject in need thereof, comprising administering to the subject a pharmaceutical composition of the application.

In another general aspect, the application relates to a method of treating a tauopathy in a subject in need thereof, comprising administering to the subject a pharmaceutical composition of the application.

In another general aspect, the application relates to a method of reducing pathological tau aggregation or spreading of tauopathy in a subject in need thereof, comprising administering to the subject a pharmaceutical composition of the application.

According to a particular aspect, the tauopathy is selected from the group consisting of familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Sträussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

According to another particular aspect, the pharmaceutical composition is administered intravenously.

According to another particular aspect, the pharmaceutical composition is delivered across the blood-brain barrier (BBB) of the subject.

According to another particular aspect, the administration reduces Fc-mediated effector function and/or does not induce rapid reticulocyte depletion.

In another general aspect, the application relates to a method of detecting the presence of PHF-tau in a biological sample from a subject, comprising contacting the biological sample with a humanized antibody or antigen-binding fragment thereof of the application, and detecting binding of the humanized antibody or antigen-binding fragment thereof to PHF-tau in the sample from the subject.

According to a particular aspect, the biological sample is a blood, serum, plasma, interstitial fluid, or cerebral spinal fluid sample.

Other aspects, features and advantages of the invention according to embodiments of the application will be apparent from the following disclosure, including the detailed description of the application and its preferred embodiments and the appended claims.

### BRIEF DESCRIPTION OF THE DRAWINGS

The foregoing summary, as well as the following detailed description of the application, will be better understood when read in conjunction with the appended drawings. It should be understood that the application is not limited to the precise embodiments shown in the drawings.

FIG. 1 is a graph showing ELISA binding of PT1B1142, humanized variant, to full-length Tau protein, in comparison to PT1B995 and PT1B585.

FIG. 2 is a graph showing the thermal stability ELISA signals of PT1B1142, humanized variant, over a range of temperatures, in comparison to PT1B995 and PT1B585.

FIG. 3 is a series of graphs showing representative SPR binding data of the parent and humanized PT66 antibodies to human full-length recombinant tau. The sensorgrams represent stepwise injection of tau protein in single-cycle kinetics mode at 2.2 nM, 6.7 nM, 20 nM, and 60 nM. The solid black overlays indicate global kinetics fitting with 1:1 Langmuir model.

FIG. 4 is a SPR sensorgram of PT1B1183 binding to full-length recombinant Tau protein. The sensorgrams represent step-wise injection in single-cycle kinetics mode of tau protein at 1.1 nM, 3.3 nM, 10 nM, and 30 nM. Association time for each Tau concentration is 3 minutes and the dissociation time is 120 minutes. The solid black overlay indicate global kinetics fitting with 1:1 Langmuir model.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the application. Such discussion is not an admission that any or all of these matters form part of the prior art with respect to any inventions disclosed or claimed.

### Definitions

Unless defined otherwise, all technical and scientific terms used herein have the same meaning commonly understood to one of ordinary skill in the art to which this application pertains. Otherwise, certain terms used herein have the meanings as set in the specification. It must be noted that as used herein and in the appended claims, the singular forms “a,” “an,” and “the” include plural reference unless the context clearly dictates otherwise.

Unless otherwise stated, any numerical value, such as a concentration or a concentration range described herein, is to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Throughout this specification and the claims which follow, unless the context requires otherwise, the word “comprise,” and variations such as “comprises” and “comprising,” will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the application can be replaced with the term “consisting of” or “consisting essentially of” to vary scopes of the disclosure.

As used herein, the term “isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if such composition is not part of the native environment of the nucleic acid, peptide, or protein. The term also embraces nucleic acids, peptides and proteins prepared by recombinant expression in a host cell as well as chemically synthesized nucleic acids.

As used herein, the term “antibody” or “immunoglobulin” is used in a broad sense and includes immunoglobulin or antibody molecules including polyclonal antibodies, monoclonal antibodies including murine, human, human-adapted, humanized and chimeric monoclonal antibodies and antibody fragments.

In general, antibodies are proteins or peptide chains that exhibit binding specificity to a specific antigen. Antibody structures are well known. Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Each of the four IgG subclasses has different biological functions known as effector functions. These effector functions are generally mediated through interaction with the Fc receptor (FcγR) and/or by binding C1q and fixing complement. Binding to FcγR can lead to antibody dependent cell mediated cytolysis or antibody-dependent cellular cytotoxicity (ADCC), whereas binding to complement factors can lead to complement mediated cell lysis or complement-dependent cytotoxicity (CDC). Antibodies of the application include those that have variations in their Fc region such that they have altered properties as compared to wild type Fc regions including, but not limited to, extended half-life, reduced or increased ADCC or CDC and silenced Fc effector functions. Accordingly, the antibodies of the application can be of any of the five major classes or corresponding sub-classes. Preferably, the antibodies of the application are IgG1, IgG2, IgG3 or IgG4. Antibody light chains of any vertebrate species can be assigned to one of two clearly distinct types, namely kappa and lambda, based on the amino acid sequences of their constant domains. Accordingly, the antibodies of the application can contain a kappa or lambda light chain constant domain. According to particular embodiments, the antibodies of the application include heavy and/or light chain constant regions from mouse antibodies or human antibodies.

In addition to the heavy and light chain constant domains, antibodies contain light and heavy chain variable regions. An immunoglobulin light or heavy chain variable region consists of a “framework” region interrupted by “antigen-binding sites.” The antigen-binding sites are defined using various terms and numbering schemes as follows:
(i) Kabat: “Complementarity Determining Regions” or “CDRs” are based on sequence variability (Wu and Kabat, J Exp Med. 132:211-50, 1970). Generally, the antigen-binding site has three CDRs in each variable region (e.g., HCDR1, HCDR2 and HCDR3 in the heavy chain variable region (VH) and LCDR1, LCDR2 and LCDR3 in the light chain variable region (VL));
(ii) Chothia: The term “hypervariable region,” “HVR” or “HV” refers to the regions of an antibody variable domain which are hypervariable in structure as defined by Chothia and Lesk (Chothia and Lesk, J Mol Biol. 196:901-17, 1987). Generally, the antigen-binding site has three hypervariable regions in each VH (H1, H2, H3) and VL (L1, L2, L3). Numbering systems as well as annotation of CDRs and HVs have been revised by Abhinandan and Martin (Abhinandan and Martin, Mol Immunol. 45:3832-9, 2008);
(iii) IMGT: Another definition of the regions that form the antigen-binding site has been proposed by Lefranc (Lefranc et al., Dev Comp Immunol. 27:55-77, 2003) based on the comparison of V domains from immunoglobulins and T-cell receptors. The International ImMunoGeneTics (IMGT) database provides a standardized numbering and definition of these regions. The correspondence between CDRs, HVs and IMGT delineations is described in Lefranc et al., 2003, Id.;
(iv) AbM: A compromise between Kabat and Chothia numbering schemes is the AbM numbering convention described by Martin (Martin A C R (2010) Antibody Engineering, eds Kontermann R, Dubel S (Springer-Verlag, Berlin), Vol 2, pp 33-51);
(v) The antigen-binding site can also be delineated based on “Specificity Determining Residue Usage” (SDRU) (Almagro, Mol Recognit. 17:132-43, 2004), where SDR, refers to amino acid residues of an immunoglobulin that are directly involved in antigen contact.

“Framework” or “framework sequence” is the remaining sequences within the variable region of an antibody other than those defined to be antigen-binding site sequences. Because the exact definition of an antigen-binding site can be determined by various delineations as described above, the exact framework sequence depends on the definition of the antigen-binding site. The framework regions (FRs) are the more highly conserved portions of variable domains. The variable domains of native heavy and light chains each comprise four FRs (FR1, FR2, FR3 and FR4, respectively) which generally adopt a beta-sheet configuration, connected by the three hypervariable loops. The hypervariable loops in each chain are held together in close proximity by the FRs and, with the hypervariable loops from the other chain, contribute to the formation of the antigen-binding site of antibodies. Structural analysis of antibodies revealed the relationship between the sequence and the shape of the binding site formed by the complementarity determining regions (Chothia et al., J. Mol. Biol. 227: 799-817, 1992; Tramontano et al., J. Mol. Biol. 215:175-182, 1990). Despite their high sequence variability, five of the six loops adopt just a small repertoire of main-chain conformations, called “canonical structures.” These conformations are first of all determined by the length of the loops and secondly by the presence of key residues at certain positions in the loops and in the framework regions that determine the conformation through their packing, hydrogen bonding or the ability to assume unusual main-chain conformations.

An “antibody” can also be a single variable domain on a heavy chain (VHH) antibody, also referred to as a heavy chain only antibody (HcAb), which are devoid of light chains and can be naturally produced by camelids or sharks. The antigen binding portion of the HcAb is comprised of a VHH fragment.

The term “recombinant antibody”, as used herein, refers to an antibody (e.g. a chimeric, humanized, or human antibody or antigen-binding fragment thereof) that is expressed by a recombinant host cell comprising nucleic acid encoding the antibody. Examples of “host cells” for producing recombinant antibodies include: (1) mammalian cells, for example, Chinese Hamster Ovary (CHO), COS, myeloma cells (including YO and NSO cells), baby hamster kidney (BHK), Hela and Vero cells; (2) insect cells, for example, sf9, sf21 and Tn5; (3) plant cells, for example plants belonging to the genus Nicotiana (e.g. Nicotiana tabacum); (4) yeast cells, for example, those belonging to the genus Saccharomyces (e.g. Saccharomyces cerevisiae) or the genus Aspergillus (e.g. Aspergillus niger); (5) bacterial cells, for example Escherichia, coli cells or Bacillus subtilis cells, etc.

An “antigen-binding fragment” of an antibody is a molecule that comprises a portion of a full-length antibody which is capable of detectably binding to the antigen, typically comprising one or more portions of at least the VH region. Antigen-binding fragments include multivalent molecules comprising one, two, three, or more antigen-binding portions of an antibody, and single-chain constructs wherein the VL and VH regions, or selected portions thereof, are joined by synthetic linkers or by recombinant methods to form a functional, antigen-binding molecule.

Antigen-binding fragments can also be a single-domain antibody (sdAb), also known as a nanobody, which is an antibody fragment consisting of a single monomeric variable antibody domain (VHH). While some antigen-binding fragments of an antibody can be obtained by actual fragmentation of a larger antibody molecule (e.g., enzymatic cleavage), most are typically produced by recombinant techniques. The antibodies of the application can be prepared as full-length antibodies or antigen-binding fragments thereof. Examples of antigen-binding fragments include Fab, Fab′, F(ab)₂, F(ab′)₂, F(ab)₃, Fv (typically the VL and VH domains of a single arm of an antibody), single-chain Fv (scFv, see e.g., Bird et al., Science 1988; 242:423-426; and Huston et al. PNAS 1988; 85:5879-5883), dsFv, Fd (typically the VH and CH1 domain), and dAb (typically a VH domain) fragments; VH, VL, VHH, and V-NAR domains; monovalent molecules comprising a single VH and a single VL chain; minibodies, diabodies, triabodies, tetrabodies, and kappa bodies (see, e.g., Ill et al., Protein Eng 1997; 10:949-57); camel IgG; IgNAR; as well as one or more isolated CDRs or a functional paratope, where the isolated CDRs or antigen-binding residues or polypeptides can be associated or linked together so as to form a functional antibody fragment. Various types of antibody fragments have been described or reviewed in, e.g., Holliger and Hudson, Nat Biotechnol 2005; 23:1126-1136; WO2005040219, and published U.S. Patent Applications 20050238646 and 20020161201. Antibody fragments can be obtained using conventional recombinant or protein engineering techniques, and the fragments can be screened for antigen-binding or other function in the same manner as are intact antibodies.

Various techniques have been developed for the production of antibody fragments. Traditionally, these fragments were derived via proteolytic digestion of full-length antibodies (see, e.g., Morimoto et al., Journal of Biochemical and Biophysical Methods, 24:107-117 (1992); and Brennan et al., Science, 229:81 (1985)). However, these fragments can now be produced directly by recombinant host cells. Alternatively, Fab′-SH fragments can be directly recovered from E. coli and chemically coupled to form F(ab′)2 fragments (Carter et al., Bio/Technology, 10:163-167 (1992)). According to another approach, F(ab′)2 fragments can be isolated directly from recombinant host cell culture. In other embodiments, the antibody of choice is a single-chain Fv fragment (scFv). See WO 1993/16185; U.S. Pat. Nos. 5,571,894; and 5,587,458. The antibody fragment may also be a “linear antibody”, e.g., as described in U.S. Pat. No. 5,641,870, for example. Such linear antibody fragments can be monospecific or bispecific.

The term “antibody derivative” as used herein refers to a molecule comprising a full-length antibody or an antigen-binding fragment thereof, wherein one or more amino acids are chemically modified or substituted. Chemical modifications that can be used in antibody derivative includes, e.g., alkylation, PEGylation, acylation, ester formation or amide formation or the like, e.g., for linking the antibody to a second molecule. Exemplary modifications include PEGylation (e.g., cysteine-PEGylation), biotinylation, radiolabeling, and conjugation with a second agent (such as a cytotoxic agent).

Antibodies herein include “amino acid sequence variants” with altered antigen-binding or biological activity. Examples of such amino acid alterations include antibodies with enhanced affinity for antigen (e.g. “affinity matured” antibodies), and antibodies with altered Fc region, if present, e.g. with altered (increased or diminished) antibody dependent cellular cytotoxicity (ADCC) and/or complement dependent cytotoxicity (CDC) (see, for example, WO 00/42072, Presta, L. and WO 99/51642, Iduosogie et al); and/or increased or diminished serum half-life (see, for example, WO00/42072, Presta, L.).

A “multispecific molecule” comprises an antibody, or an antigen-binding fragment thereof, which is associated with or linked to at least one other functional molecule (e.g. another peptide or protein such as another antibody or ligand for a receptor) thereby forming a molecule that binds to at least two different binding sites or target molecules. Exemplary multispecific molecules include bi-specific antibodies and antibodies linked to soluble receptor fragments or ligands.

The term “human antibody”, as used herein, is intended to include antibodies having variable regions in which both the framework and CDR regions are derived from (i.e., are identical or essentially identical to) human germline immunoglobulin sequences. Furthermore, if the antibody contains a constant region, the constant region also is “derived from” human germline immunoglobulin sequences. The human antibodies of the application can include amino acid residues not encoded by human germline immunoglobulin sequences (e.g., mutations introduced by random or site-specific mutagenesis in vitro or by somatic mutation in viva). However, the term “human antibody”, as used herein, is not intended to include antibodies in which CDR sequences derived from the germline of another mammalian species, such as a mouse, have been grafted onto human framework sequences.

A “humanized” antibody is a human/non-human chimeric antibody that contains a minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or non-human primate having the desired specificity, affinity, and capacity. In some instances, FR residues of the human immunoglobulin are replaced by corresponding non-human residues. Furthermore, humanized antibodies can comprise residues that are not found in the recipient antibody or in the donor antibody. These modifications are made to further refine antibody performance. In general, a humanized antibody will comprise substantially all of at least one, and typically two, variable domains, in which all or substantially all of the hypervariable loops correspond to those of a non-human immunoglobulin and all or substantially all of the FR residues are those of a human immunoglobulin sequence. The humanized antibody can optionally also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992), WO 92/02190, US Patent Application 20060073137, and U.S. Pat. Nos. 6,750,325, 6,632,927, 6,639,055, 6,548,640, 6,407,213, 6,180,370, 6,054,297, 5,929,212, 5,895,205, 5,886,152, 5,877,293, 5,869,619, 5,821,337, 5,821,123, 5,770,196, 5,777,085, 5,766,886, 5,714,350, 5,693,762, 5,693,761, 5,530,101, 5,585,089, and 5,225,539.

As used herein, the term “epitope” refers to a site on an antigen to which an immunoglobulin, antibody, or antigen-binding fragment thereof, specifically binds. Epitopes can be formed either from contiguous amino acids or from noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, G. E. Morris, Ed. (1996).

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

The ability of a target antibody to “block” the binding of a target molecule to a natural target ligand, means that the antibody, in an assay using soluble or cell-surface associated target and ligand molecules, can detectably reduce the binding of a target molecule to the ligand in a dose-dependent fashion, where the target molecule detectably binds to the ligand in the absence of the antibody.

As used herein, the term “tau” or “tau protein” refers to an abundant central and peripheral nervous system protein having multiple isoforms. In the human central nervous system (CNS), six major tau isoforms ranging in size from 352 to 441 amino acids in length exist due to alternative splicing (Hanger et al., Trends Mol Med. 15:112-9, 2009). The isoforms differ from each other by the regulated inclusion of 0-2 N-terminal inserts, and 3 or 4 tandemly arranged microtubule-binding repeats, and are referred to as 0N3R (SEQ ID NO: 53), 1N3R (SEQ ID NO: 54), 2N3R (SEQ ID NO: 55), 0N4R (SEQ ID NO: 56), 1N4R (SEQ ID NO: 57) and 2N4R (SEQ ID NO: 58). As used herein, the term “control tau” refers to the tau isoform of SEQ ID NO: 58 that is devoid of phosphorylation and other post-translational modifications. As used herein, the term “tau” includes proteins comprising mutations, e.g., point mutations, fragments, insertions, deletions and splice variants of full-length wild type tau. The term “tau” also encompasses post-translational modifications of the tau amino acid sequence. Post-translational modifications include, but are not limited to, phosphorylation. As used herein, the phrase “phosphorylated S433 of the tau protein” and similar phrases refer to a phosphorylated amino acid at a certain position, e.g., serine at position 433, of the full-length wild type tau protein.

Tau binds microtubules and regulates transport of cargo through cells, a process that can be modulated by tau phosphorylation. In AD and related disorders, abnormal phosphorylation of tau is prevalent and thought to precede and/or trigger aggregation of tau into fibrils, termed paired helical filaments (PHF). The major constituent of PHF is hyper-phosphorylated tau. As used herein, the term “paired helical filament-tau” or “PHF-tau” refers to tau aggregates in paired helical filaments. Two major regions in PHF structure are evident in electron microscopy, the fuzzy coat and the core filament; the fuzzy coat being sensitive to proteolysis and located outside of the filaments, and the protease-resistant core of filaments forming the backbone of PHFs (Wischik et al. Proc Natl Acad Sci USA. 85:4884-8, 1988).

An “isolated humanized antibody that binds PHF-tau” or an “isolated humanized anti-PHF-tau antibody”, as used herein, is intended to refer to a humanized anti-PHF-tau antibody which is substantially free of other antibodies having different antigenic specificities (for instance, an isolated humanized anti-PHF-tau antibody is substantially free of antibodies that specifically bind antigens other than PHF-tau). An isolated humanized anti-PHF-tau antibody can, however, have cross-reactivity to other related antigens, for instance from other species (such as PHF-tau species homologs).

As used herein, the term “specifically binds” or “specific binding” refers to the ability of an anti-PHF-tau antibody of the application to bind to a predetermined target with a dissociation constant (K_{D}) of about 1×10⁻⁶ M or tighter, for example, about 1×10⁻⁷ M or less, about 1×10⁻⁸ M or less, about 1×10⁻⁹ M or less, about 1×10⁻¹⁰ M or less, about 1×10⁻¹¹ M or less, about 1×10⁻¹² M or less, or about 1×10⁻¹³ M or less. The K_{D} is obtained from the ratio of Kd to Ka (i.e., Kd/Ka) and is expressed as a molar concentration (M). K_{D} values for antibodies can be determined using methods in the art in view of the present disclosure. For example, the K_{D} value of an anti-PHF-tau antibody can be determined by using surface plasmon resonance, such as by using a biosensor system, e.g., a Biacore® system, a ProteOn instrument (BioRad), a KinExA instrument (Sapidyne), ELISA or competitive binding assays known to those skilled in the art. Typically, an anti-PHF-tau antibody binds to a predetermined target (i.e. PHF-tau) with a K_{D} that is at least ten fold less than its K_{D} for a nonspecific target as measured by surface plasmon resonance using, for example, a ProteOn Instrument (BioRad). The anti-PHF-tau antibodies that specifically bind to PHF-tau can, however, have cross-reactivity to other related targets, for example, to the same predetermined target from other species (homologs), such as human or monkey, for example Macaca fascicularis (cynomolgus, cyno), Pan troglodytes (chimpanzee, chimp) or Callithrix jacchus (common marmoset, marmoset). While a monospecific antibody specifically binds one antigen or one epitope, a bispecific antibody specifically binds two distinct antigens or two distinct epitopes.

A “conjugate” as used herein refers to an antibody or protein covalently linked to one or more heterologous molecule(s), including but not limited to a therapeutic peptide or protein, an antibody, a label, or a neurological disorder drug.

As used herein the term “coupled” refers to the joining or connection of two or more objects together. When referring to chemical or biological compounds, coupled can refer to a covalent connection between the two or more chemical or biological compounds. By way of a non-limiting example, an antibody of the application can be coupled with a peptide of interest to form an antibody coupled peptide. An antibody coupled peptide can be formed through specific chemical reactions designed to conjugate the antibody to the peptide. In certain embodiments, an antibody of the application can be covalently coupled with a peptide of the application through a linker. The linker can, for example, be first covalently connected to the antibody or the peptide, then covalently connected to the peptide or the antibody.

A “linker” as used herein refers to a chemical linker or a single chain peptide linker that covalently connects two different entities. A linker can be used to connect any two of an antibody or a fragment thereof, a blood brain barrier shuttle, a fusion protein and a conjugate of the present application. The linker can connect, for example, the VH and VL in scFv, or the humanized antibody or antigen-binding fragment thereof with a second molecule, such as a second antibody. In some embodiments, if the monovalent binding entity comprises a scFv directed to CD98, preferably human CD98hc, and the therapeutic molecule comprises an antibody directed to Tau, then the linker can connect the scFv to the antibody directed to Tau. In some embodiments, if the monovalent binding entity comprises a scFv directed to TfR, preferably huTfR1, and the therapeutic molecule comprises an antibody directed to a CNS target, such as Tau, then the linker can connect the scFv to the antibody directed to Tau. Single chain peptide linkers, comprised of from 1 to 25 amino acids, such as 1, 2, 3, 4, 5, 6, 7, 8, 9, 10, 11, 12, 13, 14, 15, 16, 17, 18, 19, 20, 21, 22, 23, 24, or 25 amino acids, joined by peptide bonds, can be used. In certain embodiments, the amino acids are selected from the twenty naturally occurring amino acids. In certain other embodiments, one or more of the amino acids are selected from glycine, alanine, proline, asparagine, glutamine and lysine. Chemical linkers, such as a hydrocarbon linker, a polyethylene glycol (PEG) linker, a polypropylene glycol (PPG) linker, a polysaccharide linker, a polyester linker, a hybrid linker consisting of PEG and an embedded heterocycle, and a hydrocarbon chain can also be used.

The term “CD98” or “CD98hc” as used herein, refers to an integral membrane protein consisting of a cluster of differentiation 98 heavy chain (CD98hc) that links to any of multiple light chains by a disulfide bond. When associated with LAT1 or LAT2, the heterodimer transporter complexes behave as obligatory amino acid exchangers. CD98hc has a molecular weight of about 80 kDa. Preferably, the CD98hc is a human CD98hc (huCD98hc). huCD98hc is encoded by the SLC3A2 gene.

The term “transferrin receptor” or “TfR,” as used herein, refers to a cell surface receptor necessary for cellular iron uptake by the process of receptor-mediated endocytosis. carrier protein for transferrin. TfR is involved in iron uptake in vertebrates and is regulated in response to intracellular iron concentration. It imports iron by internalizing the transferrin-iron complex through receptor-mediated endocytosis. Two transferrin receptors in humans, transferrin receptor 1 and transferrin receptor 2, have been characterized. Both these receptors are transmembrane glycoproteins. TfR1 is a high affinity ubiquitously expressed receptor. TfR2 binds to transferrin with a 25-30-fold lower affinity than TfR1. The expression of TfR2 is restricted to certain cell types and is unaffected by intracellular iron concentrations. In one embodiment, the TfR is a human TfR comprising the amino acid sequence as in Schneider et al. Nature 311: 675-678 (1984), for example. It can have a molecular weight of about 180,000 Dalton, having two subunits each of apparent molecular weight of about 90,000 Dalton. Preferably, the TfR is a human TfR1 (huTfR1).

The phrases “sequence identity” or “percent (%) sequence identity” or “% identity” or “% identical to” when used with reference to an amino acid sequence describe the number of matches (“hits”) of identical amino acids of two or more aligned amino acid sequences as compared to the number of amino acid residues making up the overall length of the amino acid sequences. In other terms, using an alignment, for two or more sequences the percentage of amino acid residues that are the same (e.g. 90%, 91%, 92%, 93%, 94%, 95%, 97%, 98%, 99%, or 100% identity over the full-length of the amino acid sequences) may be determined, when the sequences are compared and aligned for maximum correspondence as measured using a sequence comparison algorithm as known in the art, or when manually aligned and visually inspected. The sequences which are compared to determine sequence identity may thus differ by substitution(s), addition(s) or deletion(s) of amino acids. Suitable programs for aligning protein sequences are known to the skilled person. The percentage sequence identity of protein sequences can, for example, be determined with programs such as CLUSTALW, Clustal Omega, FASTA or BLAST, e.g. using the NCBI BLAST algorithm (Altschul S F, et al (1997), Nucleic Acids Res. 25:3389-3402).

The term “substantially identical” in the context of two amino acid sequences means that the sequences, when optimally aligned, such as by the programs GAP or BESTFIT using default gap weights, share at least about 50 percent sequence identity. Typically sequences that are substantially identical will exhibit at least about 60, at least about 70, at least about 80, at least about 90, at least about 95, at least about 98, or at least about 99 percent sequence identity.

“Polypeptide” or “protein” means a molecule that comprises at least two amino acid residues linked by a peptide bond to form a polypeptide. Small polypeptides of less than 50 amino acids may be referred to as “peptides”.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the application. The “host cell” can be any type of cell, e.g., a primary cell, a cell in culture, or a cell from a cell line. In one embodiment, a “host cell” is a cell transfected with a nucleic acid molecule of the application. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed humanized antibody or antigen-binding fragment thereof that binds PHF-tau can be within the cytoplasm of a host cell, into the extracellular milieu such as the growth medium of a cell culture, or anchored to the cell membrane.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the application or the biological activity of a composition according to the application. According to particular embodiments, in view of the present disclosure, any pharmaceutically acceptable carrier suitable for use in an antibody pharmaceutical composition can be used in the application.

As used herein, the term “subject” refers to an animal, and preferably a mammal. According to particular embodiments, the subject is a mammal including a non-primate (e.g., a camel, donkey, zebra, cow, pig, horse, goat, sheep, cat, dog, rat, rabbit, guinea pig or mouse) or a primate (e.g., a monkey, chimpanzee, or human). In particular embodiments, the subject is a human.

The term “administering” with respect to the methods of the application, means a method for therapeutically or prophylactically preventing, treating or ameliorating a syndrome, disorder or disease as described herein by using an antibody, antigen-binding fragment thereof, or a conjugate of the application, or a form, composition or medicament thereof. Such methods include administering an effective amount of said antibody, antigen-binding fragment thereof, or conjugate, or a form, composition or medicament thereof at different times during the course of a therapy or concurrently in a combination form. The methods of the application are to be understood as embracing all known therapeutic treatment regimens.

As used herein, the term “therapeutically effective amount” refers to an amount of an active ingredient or component that elicits the desired biological or medicinal response in a subject. A therapeutically effective amount can be determined empirically and in a routine manner, in relation to the stated purpose. For example, in vitro assays can optionally be employed to help identify optimal dosage ranges. Selection of a particular effective dose can be determined (e.g., via clinical trials) by those skilled in the art based upon the consideration of several factors, including the disease to be treated or prevented, the symptoms involved, the patient's body mass, the patient's immune status and other factors known by the skilled artisan. The precise dose to be employed in the formulation will also depend on the route of administration, and the severity of disease, and should be decided according to the judgment of the practitioner and each patient's circumstances. Effective doses can be extrapolated from dose-response curves derived from in vitro or animal model test systems.

As used herein, the terms “treat,” “treating,” and “treatment” are all intended to refer to an amelioration or reversal of at least one measurable physical parameter related to a tauopathy which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the tauopathy. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

As used herein a “tauopathy” encompasses any neurodegenerative disease that involves the pathological aggregation of tau within the brain. In addition to familial and sporadic AD, other exemplary tauopathies are frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Sträussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, and chronic traumatic encephalopathy, such as dementia pugulistica (boxing disease) (Morris et al., Neuron, 70:410-26, 2011).

The “blood-brain barrier” or “BBB” refers a physiological barrier between the peripheral circulation and the brain and spinal cord which is formed by tight junctions within the brain capillary endothelial plasma membranes, creating a tight barrier that restricts the transport of molecules into the brain. The BBB can restrict the transport of even very small molecules such as urea (60 Daltons) into the brain. Examples of the BBB include the BBB within the brain, the blood-spinal cord barrier within the spinal cord, and the blood-retinal barrier within the retina, all of which are contiguous capillary barriers within the CNS. The BBB also encompasses the blood-CSF barrier (choroid plexus) where the barrier is comprised of ependymal cells rather than capillary endothelial cells.

A “blood-brain barrier receptor” (abbreviated “R/BBB” herein) is an extracellular membrane-linked receptor protein expressed on brain endothelial cells which is capable of transporting molecules across the BBB or be used to transport exogenous administrated molecules. Examples of R/BBB include, but are not limited to, Large neutral Amino acid Transporter (LAT) complex, including CD98 component, transferrin receptor (TfR), insulin receptor, insulin-like growth factor receptor (IGF-R), low density lipoprotein receptors including without limitation low density lipoprotein receptor-related protein 1 (LRP1) and low density lipoprotein receptor-related protein 8 (LRP8), and heparin-binding epidermal growth factor-like growth factor (HB-EGF). An exemplary R/BBB herein is CD98hc or transferrin receptor (TfR).

The “central nervous system” or “CNS” refers to the complex of nerve tissues that control bodily function, and includes the brain and spinal cord.

A “neurological disorder” as used herein refers to a disease or disorder which affects the CNS and/or which has an etiology in the CNS. Exemplary CNS diseases or disorders include, but are not limited to, neuropathy, amyloidosis, cancer, an ocular disease or disorder, viral or microbial infection, inflammation, ischemia, neurodegenerative disease, seizure, behavioral disorders, and a lysosomal storage disease. For the purposes of this application, the CNS will be understood to include the eye, which is normally sequestered from the rest of the body by the blood-retina barrier. Specific examples of neurological disorders include, but are not limited to, neurodegenerative diseases (including, but not limited to, Lewy body disease, postpoliomyelitis syndrome, Shy-Draeger syndrome, olivopontocerebellar atrophy, Parkinson's disease, multiple system atrophy, striatonigral degeneration, spinocerebellar ataxia, spinal muscular atrophy), tauopathies (including, but not limited to, Alzheimer disease and supranuclear palsy), prion diseases (including, but not limited to, bovine spongiform encephalopathy, scrapie, Creutz-feldt-Jakob syndrome, kuru, Gerstmann-Sträussler-Scheinker disease, chronic wasting disease, and fatal familial insomnia), bulbar palsy, motor neuron disease, and nervous system heterodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), cancer (e.g. of the CNS and/or brain, including brain metastases resulting from cancer elsewhere in the body).

A “neurological disorder drug” is a drug or therapeutic agent useful in treating or ameliorating the effects of one or more neurological disorder(s). Neurological disorder drugs of the application include, but are not limited to, small molecule compounds, antibodies, peptides, proteins, natural ligands of one or more CNS target(s), modified versions of natural ligands of one or more CNS target(s), aptamers, inhibitory nucleic acids (i.e., small inhibitory RNAs (siRNA) and short hairpin RNAs (shRNA)), ribozymes, or active fragments of any of the foregoing. Exemplary neurological disorder drugs of the application are described herein and include, but are not limited to: antibodies, aptamers, proteins, peptides, inhibitory nucleic acids and small molecules and active fragments of any of the foregoing that either are themselves or specifically recognize and/or act upon (i.e., inhibit, activate, or detect) a CNS antigen or target molecule such as, but not limited to, amyloid precursor protein or portions thereof, amyloid beta, beta-secretase, gamma-secretase, tau, alpha-synuclein, parkin, huntingtin, DR6, presenilin, ApoE, glioma or other CNS cancer markers, and neurotrophins Non-limiting examples of neurological disorder drugs and the corresponding disorders they may be used to treat: Brain-derived neurotrophic factor (BDNF), Chronic brain injury (Neurogenesis), Fibroblast growth factor 2 (FGF-2), Anti-Epidermal Growth Factor Receptor Brain cancer, (EGFR)-antibody, Glial cell-line derived neural factor Parkinson's disease, (GDNF), Brain-derived neurotrophic factor (BDNF) Amyotrophic lateral sclerosis, depression, Lysosomal enzyme Lysosomal storage disorders of the brain, Ciliary neurotrophic factor (CNTF) Amyotrophic lateral sclerosis, Neuregulin-1 Schizophrenia, Anti-HER2 antibody (e.g. trastuzumab) Brain metastasis from HER2-positive cancer.

A “target antigen” or “brain target,” as used herein, refers to an antigen and/or molecule expressed in the CNS, including the brain, which can be targeted with an antibody or small molecule. Examples of such antigens and/or molecules include, without limitation: beta-secretase 1 (BACE1), amyloid beta (Abeta), epidermal growth factor receptor (EGFR), human epidermal growth factor receptor 2 (HER2), Tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), and caspase 6. In some embodiments, the target antigen is BACE1. In some preferred embodiments, the target antigen is Tau.

The term “pharmaceutical formulation” refers to a preparation which is in such form as to permit the biological activity of an active ingredient contained therein to be effective, and which contains no additional components which are unacceptably toxic to a subject to which the formulation would be administered.

As used herein, “pharmaceutically acceptable carrier or diluent” means any substance suitable for use in administering to an individual. For example, a pharmaceutically acceptable carrier can be a sterile aqueous solution, such as phosphate buffer saline (PBS) or water-for-injection.

As used herein, “pharmaceutically acceptable salts” means physiologically and pharmaceutically acceptable salts of compounds, such as oligomeric compounds or oligonucleotides, i.e., salts that retain the desired biological activity of the parent compound and do not impart undesired toxicological effects thereto.

Pharmaceutically acceptable acidic/anionic salts for use in the application include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide. Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid. Pharmaceutically acceptable basic/cationic salts include, and are not limited to aluminum, 2-amino-2-hydroxymethyl-propane-1,3-diol (also known as tris(hydroxymethyl)aminomethane, tromethane or “TRIS”), ammonia, benzathine, t-butylamine, calcium, chloroprocaine, choline, cyclohexylamine, diethanolamine, ethylenediamine, lithium, L-lysine, magnesium, meglumine, N-methyl-D-glucamine, piperidine, potassium, procaine, quinine, sodium, triethanolamine, or zinc.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

### Humanized Anti-PHF-Tau Antibodies

In one general aspect, the application relates to isolated humanized antibodies or antigen-binding fragments thereof that bind PHF-tau. Such anti-PHF-tau antibodies can have the properties of binding a phosphorylated epitope on PHF-tau or binding to a non-phosphorylated epitope on PHF-tau. Anti-PHF-tau antibodies can be useful as therapeutics, and as research or diagnostic reagents to detect PHF-tau in biological samples, for example in tissues or cells.

According to a particular aspect, the application relates to an isolated humanized antibody or an antigen-binding fragment thereof that binds to a tau protein at an epitope in the C-terminus domain of the tau protein. In some embodiments, the isolated humanized antibody or antigen-binding fragment thereof binds to a tau protein at an epitope of the tau protein having or within the amino acid sequence of SEQ ID NO: 1, wherein the antibody or antigen-binding fragment thereof binds PHF-tau, preferably human PHF-tau. Preferably, the isolated humanized antibody or antigen-binding fragment thereof binds to a tau protein at an epitope of the tau protein consisting of or within the amino acid sequence of SEQ ID NO: 1, wherein the antibody or antigen-binding fragment thereof binds PHF-tau, preferably human PHF-tau.

In some embodiments, the epitope of the tau protein comprises one or more of phosphorylated T427, phosphorylated S433 and phosphorylated S435 of the tau protein, but does not comprise all of phosphorylated T427, phosphorylated S433 and phosphorylated S435.

Humanized antibodies have variable region framework residues substantially from a human antibody (termed an acceptor antibody) and complementarity determining regions substantially from a mouse-antibody, (referred to as the donor immunoglobulin). See Queen et al., Proc. Natd. Acad. Sci. USA. 86:10029-10033, 1989, WO 90/07861, U.S. Pat. Nos. 5,693,762, 5,693,761, 5,585,089, 5,530,101, and 5,225,539. The constant region(s), if present, are also substantially or entirely from a human immunoglobulin. The human variable domains are usually chosen from human antibodies whose framework sequences exhibit a high degree of sequence identity with the murine variable region domains from which the CDRs were derived. The heavy and light chain variable region framework residues can be derived from the same or different human antibody sequences. The human antibody sequences can be the sequences of naturally occurring human antibodies or can be consensus sequences of several human antibodies. See WO 92/22653. Certain amino acids from the human variable region framework residues are selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid should usually be substituted by the equivalent framework amino acid from the mouse antibody when it is reasonably expected that the amino acid: (1) noncovalently binds antigen directly, (2) is adjacent to a CDR region, (3) otherwise interacts with a CDR region (e.g. is within about 6 angstroms of a CDR region), or (4) participates in the VL-VH interface.

Other candidates for substitution are acceptor human framework amino acids that are unusual for a human immunoglobulin at that position. These amino acids can be substituted with amino acids from the equivalent position of the mouse donor antibody or from the equivalent positions of more typical human immunoglobulins. The variable region frameworks of humanized immunoglobulins usually show at least 85% sequence identity to a human variable region framework sequence or consensus of such sequences. Exemplary framework substitutions include, e.g., those described in Example 1 below.

Antibody humanization can be accomplished using well known methods, such as specificity determining residues resurfacing (SDRR) (US2010/0261620), resurfacing (Padlan et al., Mol. Immunol. 28:489-98, 1991), super humanization (WO 04/006955) and human string content optimization (U.S. Pat. No. 7,657,380). Human framework sequences useful for grafting or humanization can be selected from relevant databases by those skilled in the art. The selected frameworks can further be modified to preserve or enhance binding affinity by techniques such as those disclosed in Queen et al., 1989, Id. According to particular embodiments, methods for humanizing anti-PHF-tau antibodies from mouse parental antibodies include those described in Example 1 below.

Antibodies of the present application can be produced by a variety of techniques, for example by the hybridoma method (Kohler and Milstein, Nature. 256:495-7, 1975). Chimeric monoclonal antibodies containing a light chain and heavy chain variable region derived from a donor antibody (typically murine) in association with light and heavy chain constant regions derived from an acceptor antibody (typically another mammalian species such as human) can be prepared by a method disclosed in U.S. Pat. No. 4,816,567. CDR-grafted monoclonal antibodies having CDRs derived from a non-human donor immunoglobulin (typically murine) and the remaining immunoglobulin-derived parts of the molecule being derived from one or more human immunoglobulins can be prepared by techniques known to those skilled in the art such as that disclosed in U.S. Pat. No. 5,225,539. Fully human monoclonal antibodies lacking any non-human sequences can be prepared from human immunoglobulin transgenic mice by techniques referenced in Lonberg et al., Nature. 368:856-9, 1994; Fishwild et al., Nat Biotechnol. 14:845-51, 1996; and Mendez et al., Nat Genet. 15:146-56, 1997. Human monoclonal antibodies can also be prepared and optimized from phage display libraries (see, e.g., Knappik et al., J Mol Biol. 296:57-86, 2000; Krebs et al., J Immunol Methods. 254:67-84, 2001; Shi et al., J Mol Biol. 397:385-96, 2010).

In some embodiments, the isolated humanized antibody or antigen-binding fragment thereof comprises immunoglobulin heavy chain complementarity determining regions (HCDRs) HCDR1, HCDR2 and HCDR3 having the polypeptide sequences of SEQ ID NOs: 4, 5 and 6, respectively; and immunoglobulin light chain complementarity determining regions (LCDRs) LCDR1, LCDR2 and LCDR3 having the polypeptide sequences of SEQ ID NOs: 7 or 14, 8 and 9, respectively; wherein the antibody or antigen-binding fragment thereof binds PHF-tau, preferably human PHF-tau, and wherein the framework regions in the heavy chain variable region domain and in the light chain variable region domain comprise amino acid sequences from a human immunoglobulin.

In some embodiments, the isolated humanized antibody or antigen-binding fragment thereof comprises a heavy chain variable region having a polypeptide sequence at least 80%, preferably at least 85% or 90%, more preferably at least 95%, and most preferably 100%, identical to SEQ ID NO: 12 or 18, or a light chain variable region having a polypeptide sequence at least 80%, preferably at least 85% or 90%, more preferably at least 95%, and most preferably 100%, identical to SEQ ID NO: 13, 19, or 23.

In some embodiments, the isolated humanized antibody or antigen-binding fragment thereof comprises a heavy chain variable region having the polypeptide sequence at least 80%, preferably at least 85% or 90%, more preferably at least 95%, and most preferably 100%, identical to SEQ ID NO: 12 or 18, and a light chain variable region having the polypeptide sequence at least 80%, preferably at least 85% or 90%, more preferably at least 95%, and most preferably 100%, identical to SEQ ID NO: 13, 19, or 23.

In some embodiments, the isolated humanized antibody or antigen-binding fragment thereof comprises a heavy chain variable region having the polypeptide sequence of SEQ ID NO: 12, and a light chain variable region having the polypeptide sequence of SEQ ID NO: 13. In some embodiments, the isolated humanized antibody or antigen-binding fragment thereof comprises a heavy chain variable region having the polypeptide sequence of SEQ ID NO: 18, and a light chain variable region having the polypeptide sequence of SEQ ID NO: 19. In some embodiments, the isolated humanized antibody or antigen-binding fragment thereof comprises a heavy chain variable region having the polypeptide sequence of SEQ ID NO: 12, and a light chain variable region having the polypeptide sequence of SEQ ID NO: 23.

In some embodiments, the isolated humanized antibody or antigen-binding fragment thereof comprises:
(a) a first heavy chain having the polypeptide sequence of SEQ ID NO: 15 or 20; (b) two light chains each independently having the polypeptide sequence of SEQ ID NO: 16, 21, or 24, or 60; and
(c) a second heavy chain having the polypeptide sequence of SEQ ID NO: 17 or 22.

According to another particular aspect, the isolated humanized antibody or antigen-binding fragment thereof comprises:
(b) a heavy chain having the polypeptide sequence of SEQ ID NO: 61 or 62; and
(c) a light chain having the polypeptide sequence of SEQ ID NO: 16 or 24, or 21 or 60, respectively.

In some embodiments, the isolated humanized antibody or antigen-binding fragment thereof comprises a first heavy chain having the polypeptide sequence of SEQ ID NO: 15, two light chains each having the polypeptide sequence of SEQ ID NO: 16, and a second heavy chain having the polypeptide sequence of SEQ ID NO: 17. In some embodiments, the isolated humanized antibody or antigen-binding fragment thereof comprises a first heavy chain having the polypeptide sequence of SEQ ID NO: 20, two light chains each having the polypeptide sequence of SEQ ID NO: 21, and a second heavy chain having the polypeptide sequence of SEQ ID NO: 22. In some embodiments, the isolated humanized antibody or antigen-binding fragment thereof comprises a first heavy chain having the polypeptide sequence of SEQ ID NO: 15, two light chains each having the polypeptide sequence of SEQ ID NO: 24, and a second heavy chain having the polypeptide sequence of SEQ ID NO: 17. In some embodiments, the isolated humanized antibody or antigen-binding fragment thereof comprises a first heavy chain having the polypeptide sequence of SEQ ID NO: 20, two light chains each having the polypeptide sequence of SEQ ID NO: 60, and a second heavy chain having the polypeptide sequence of SEQ ID NO: 22.

In some embodiments, the isolated humanized antibody comprises two heavy chains each having the polypeptide sequence of SEQ ID NO: 61 and two light chains each having the polypeptide sequence of SEQ ID NO: 16 or 24. In some embodiments, the isolated humanized antibody comprises two heavy chains each having the polypeptide sequence of SEQ ID NO: 62 and two light chains each having the polypeptide sequence of SEQ ID NO: 21 or 60.

In some embodiments, the isolated monoclonal antibody or antigen-binding fragment thereof, wherein the antibody or antigen-binding fragment binds to PHF-tau with a dissociation constant (K_{D}) of 5×10⁻⁹ M or less, preferably a K_{D} of 1×10⁻⁹ M or less or 1×10⁻¹⁰ M or less, wherein the K_{D} is measured by surface plasmon resonance analysis, such as by using a Biacore or ProteOn system.

The functional activity of humanized antibodies and antigen-binding fragments thereof that bind PHF-tau can be characterized by methods known in the art and as described herein. Methods for characterizing antibodies and antigen-binding fragments thereof that bind PHF-tau include, but are not limited to, affinity and specificity assays including Biacore, ELISA, and FACS analysis; immunohistochemistry analysis; in vitro cellular assays and in vivo injection assays to determine the efficacy of the antibodies in inhibiting tau seeding; cell cytotoxicity assays to detect the presence of antibody-dependent cell-mediated cytotoxicity (ADCC), and complement dependent cytotoxicity (CDC) activity of the antibodies; etc. According to particular embodiments, methods for characterizing antibodies and antigen-binding fragments thereof that bind PHF-tau include those described in the Examples below. An exemplary mouse parental antibody of humanized antibodies binding PHF-tau but not control tau is antibody PT3, which is described in U.S. Pat. No. 9,371,376, the content of which is incorporated herein by reference in its entirety.

Several well-known methodologies can be employed to determine the binding epitope of the antibodies of the application. For example, when the structures of both individual components are known, in silico protein-protein docking can be carried out to identify compatible sites of interaction. Hydrogen-deuterium (H/D) exchange can be carried out with the antigen and antibody complex to map regions on the antigen that are bound by the antibody. Segment and point mutagenesis of the antigen can be used to locate amino acids important for antibody binding. The co-crystal structure of an antibody-antigen complex is used to identify residues contributing to the epitope and paratope.

Antibodies of the application can be bispecific or multispecific. An exemplary bispecific antibody can bind two distinct epitopes on PHF-tau or can bind PHF-tau and amyloid beta (Abeta). Another exemplary bispecific antibody can bind PHF-tau and an endogenous blood-brain barrier transcytosis receptor such as insulin receptor, transferrin receptor, insulin-like growth factor-1 receptor, and lipoprotein receptor. An exemplary antibody is of IgG1 type.

Immune effector properties of the antibodies of the application can be enhanced or silenced through Fc modifications by techniques known to those skilled in the art. For example, Fc effector functions such as C1q binding, complement dependent cytotoxicity (CDC), antibody-dependent cell-mediated cytotoxicity (ADCC), phagocytosis, down regulation of cell surface receptors (e.g., B cell receptor; BCR), etc. can be provided and/or controlled by modifying residues in the Fc responsible for these activities. Pharmacokinetic properties can also be enhanced by mutating residues in the Fc domain that extend antibody half-life (Strohl, Curr Opin Biotechnol. 20:685-91, 2009).

An anti-PHF-tau antibody can have no or minimal effector function, but retains its ability to bind FcRn, the binding of which can be a primary means by which antibodies have an extended in vivo half-life. Binding of FcγR or complement (e.g., C1q) to an antibody is caused by defined protein-protein interactions at the so-called Fc part binding site. Such Fc part binding sites are known in the art. Such Fc part binding sites include, e.g., the amino acids L234, L235, D270, N297, E318, K320, K322, P331, and P329 (numbering according to EU index of Kabat).

In some embodiments, an anti-PHF-tau antibody contains one or more substitutions in one or more Fc part binding sites to eliminate the effector function. For example, an anti-PHF-tau antibody can contain a Fc region containing one or more of the following substitutions: substitution of proline for glutamate at residue 233, alanine or valine for phenylalanine at residue 234 and alanine or glutamate for leucine at residue 235 (EU numbering, Kabat, E. A. et al. (1991) Sequences of Proteins of Immunological Interest, 5th Ed. U.S. Dept. of Health and Human Services, Bethesda, Md., NIH Publication no. 91-3242). Preferably, the antibody of interest contains one, two or three mutations of L234A, L235A and P331S (EU numbering, Kabat).

Antibodies of subclass IgG1, IgG2, and IgG3 usually show complement activation including C1q and C3 binding, whereas IgG4 does not activate the complement system and does not bind C1q and/or C3. Human IgG4 Fc region has reduced ability to bind FcγR and complement factors compared to other IgG sub-types. Preferably, an anti-PHF-tau antibody of the application comprises a Fc region derived from human IgG4 Fc region. More preferably, the Fc region contains human IgG4 Fc region having substitutions that eliminate effector function. For example, removing the N-linked glycosylation site in the IgG4 Fc region by substituting Ala for Asn at residue 297 (EU numbering) is another way to ensure that residual effector activity is eliminated.

Additionally, antibodies of the application can be post-translationally modified by processes such as glycosylation, isomerization, deglycosylation or non-naturally occurring covalent modification such as the addition of polyethylene glycol moieties and lipidation. Such modifications can occur in vivo or in vitro. For example, the antibodies of the application can be conjugated to polyethylene glycol (PEGylated) to improve their pharmacokinetic profiles. Conjugation can be carried out by techniques known to those skilled in the art. Conjugation of therapeutic antibodies with PEG has been shown to enhance pharmacodynamics while not interfering with function (Knight et al., Platelets. 15:409-18, 2004; Leong et al., Cytokine. 16:106-19, 2001; Yang et al., Protein Eng. 16:761-70, 2003).

In another general aspect, the application relates to an isolated polynucleotide encoding a humanized antibody or antigen-binding fragment thereof of the application. It will be appreciated by those skilled in the art that the coding sequence of a protein can be changed (e.g., replaced, deleted, inserted, etc.) without changing the amino acid sequence of the protein.

Accordingly, it will be understood by those skilled in the art that nucleic acid sequences encoding humanized antibodies or antigen-binding fragments thereof of the application can be altered without changing the amino acid sequences of the proteins. Exemplary isolated polynucleotides are polynucleotides encoding polypeptides comprising the immunoglobulin heavy chain and light chains described in the Examples (e.g., SEQ ID NOs: 15-17, 20-22, 24, and 60-62) and polynucleotides encoding polypeptides comprising the heavy chain variable regions (VH) and light chain variable regions (VL) (e.g., SEQ ID NOs: 12, 13, 18, 19, 23, and 59). Other polynucleotides which, given the degeneracy of the genetic code or codon preferences in a given expression system, encode the antibodies of the application are also within the scope of the application. The isolated nucleic acids of the present application can be made using well known recombinant or synthetic techniques. DNA encoding the monoclonal antibodies is readily isolated and sequenced using methods known in the art. Where a hybridoma is produced, such cells can serve as a source of such DNA. Alternatively, display techniques wherein the coding sequence and the translation product are linked, such as phage or ribosomal display libraries, can be used.

In another general aspect, the application relates to a vector comprising an isolated polynucleotide encoding a humanized antibody or antigen-binding fragment thereof of the application. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments of the application.

In another general aspect, the application relates to a host cell comprising an isolated polynucleotide encoding a humanized antibody or antigen-binding fragment thereof of the application. Any host cell known to those skilled in the art in view of the present disclosure can be used for recombinant expression of antibodies or antigen-binding fragments thereof of the application. Such host cells can be eukaryotic cells, bacterial cells, plant cells or archaeal cells. Exemplary eukaryotic cells can be of mammalian, insect, avian or other animal origins. Mammalian eukaryotic cells include immortalized cell lines such as hybridomas or myeloma cell lines such as SP2/0 (American Type Culture Collection (ATCC), Manassas, Va., CRL-1581), NSO (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATTC CRL-TIB-196). Other useful cell lines include those derived from Chinese Hamster Ovary (CHO) cells such as CHO-K1 SV (Lonza Biologics), CHO-K1 (ATCC CRL-61, Invitrogen) or DG44.

In another general aspect, the application relates to a method of producing a humanized antibody or antigen-binding fragment thereof of the application, comprising culturing a cell comprising a polynucleotide encoding the humanized antibody or antigen-binding fragment thereof under conditions to produce a humanized antibody or antigen-binding fragment thereof of the application, and recovering the antibody or antigen-binding fragment thereof from the cell or cell culture (e.g., from the supernatant). Expressed antibodies or antigen-binding fragments thereof can be harvested from the cells and purified according to conventional techniques known in the art.

Conjugates and Fusion Constructs with Anti-CD98 or Anti-TfR Antibodies or Antigen-Binding Fragments Thereof

While the blood-brain barrier (BBB) prevents harmful substances from entering the brain and is essential for brain homeostasis, it presents a formidable obstacle for efficiently delivering drugs to the brain. Numerous approaches have been studied to improve the brain delivery of therapeutic monoclonal antibodies (mAbs), including the use of receptor-mediated transcytosis (RMT). RMT utilizes abundantly expressed receptors on the luminal side of the BBB for transport through brain endothelial cells. Previous efforts to generate a clinically feasible platform for delivery of therapeutic mAbs into the brain have been focused on antibody engineering to increase the efficiency of transcytosis, with gains made through observations on valency of binding, pH dependency and affinity (reviewed in Goulatis et al., 2017, Curr Opin Struct Biol 45: 109-115). However, translation into non-human primates (NHPs) and the clinic has been limited by rapid peripheral clearance from target-mediated drug disposition (TMDD) and safety from acute reticulocyte depletion (Gadkar K, et al. Eur J Pharm Biopharm. 2016; 101: 53-61).

In a general aspect, the application relates to a humanized anti-PHF-tau antibody or antigen-binding fragment thereof coupled to an optimized platform for brain delivery. The platform utilizes a CD98 or TfR binding molecule, in particular, an antibody or antigen-binding fragment thereof that binds to CD98 or TfR, preferably a human CD98 heavy chain (huCD98hc) or human TfR1 (huTfR1). The inventors discovered, unexpectedly, the optimal values are not simply the fastest on-rate kₐ values and the slowest off-rate k_{d} values as one might expect in typical antibody-target interactions. That is, for this system, one would not necessarily want to use a molecule that “binds” and associates with CD98 or TfR at a relatively high rate and then dissociates from the CD98 or TfR more slowly to have the longest life span of the antibody-target complex. Instead, a neutral off-rate that is neither too fast nor too slow is required for optimal brain PK and PD of an agent (such as an mAb) to be efficiently delivered by the anti-CD98 or anti-TfR antibody or antigen binding fragment thereof. It was also discovered that engineered antibody constant region with increased binding to the neonatal Fc receptor (FcRn) resulted in decreased peripheral clearance and enhancement in brain concentration.

Additional Fc mutations are introduced to abolish binding to Fc gamma receptors (FcγR) and avoid effector function mediated toxicity. When coupled with a high affinity anti-Tau binding mAb, these mutations prevent effector function mediated toxicity in the periphery while maintaining antibody dependent phagocytosis (ADP) through a novel, non-FcγR mechanism for microglial uptake and target degradation. This mechanism is dependent upon internalization through CD98 or TfR and is more efficient in promoting target degradation than traditional FcγR mediated ADP without the stimulating the secretion of pro-inflammatory cytokines.

Preferably, an anti-CD98 or anti-TfR antibody or antigen binding fragment thereof of the application is pH-sensitive, e.g., it has different binding affinities to CD98 or TfR at different pHs. For example, an anti-CD98 or anti-TfR antibody of the application can bind to CD98 orTfR at a neutral pH, such as physiological pH (e.g., pH 7.4), with high affinity, but upon internalization into an endosomal compartment, dissociates from CD98 or TfR at an acidic pH, such as the relatively lower pH (pH 5-6.0). Affinity is a measure of the strength of binding between two moieties, e.g., an antibody and an antigen. Affinity can be expressed in several ways. One way is in terms of the dissociation constant (K_{D}) of the interaction. K_{D} can be measured by routine methods, include equilibrium dialysis or by directly measuring the rates of antigen-antibody dissociation and association, the k_{off} (kd or k_{dis}) and kₒₙ(or ka) rates, respectively (see e.g., Nature, 1993 361:186-87). The ratio of k_{off}/kₒₙ cancels all parameters not related to affinity, and is equal to the dissociation constant K_{D} (see, generally, Davies et al., Annual Rev Biochem, 1990 59:439-473). Thus, a smaller K_{D} means a higher affinity. Another expression of affinity is Kₐ, which is the inverse of K_{D}, or kₒₙ/k_{off}. Thus, a higher Kₐ means a higher affinity. For example, a CD98 or TfR antibody or antigen binding fragment thereof for use in a composition and/or method of the application can be an antibody or fragment thereof that binds to a CD98 or TfR with a K_{D} of 1 nanomolar (nM, 10⁻⁹ M) or more at a neutral pH (e.g., pH 6.8-7.8), such as a physiological pH (e.g., pH 7.4), and dissociates from CD98 or TfR with a k_{dis} of 10⁻⁴ sec⁻¹ or more at an acidic pH (e.g., pH 4.5-6.5), such as pH 5.0.

Accordingly, a general aspect of the application relates to a humanized anti-PHF-tau antibody or antigen-binding fragment thereof coupled to an anti-CD98 or anti-TfR antibody or antigen-binding fragment thereof for delivery to the brain of a subject in need thereof, wherein the anti-CD98 or anti-TfR antibody or antigen-binding fragment thereof binds to a CD98, preferably a CD98hc, more preferably a human CD98hc, or a TfR, preferably a TfR1, more preferably a human TfR1, with a dissociation constant K_{D} of at least 1 nM, preferably 1 nM to 500 nM, at neutral pH and an off-rate constant k_{d} of at least 10⁻⁴ sec⁻¹, preferably 10⁻⁴ to 10⁻¹ sec 1, at an acidic pH, preferably pH 5.

In some embodiments, the anti-CD98 or anti-TfR antibody or antigen-binding fragment thereof of the application has an off-rate constant k_{d} of 2×10⁻² to 2×10⁻⁴ sec⁻¹, such as 2×10⁻², 1×10⁻², 9×10⁻³, 8×10⁻³, 7×10⁻³, 6×10⁻³, 5×10⁻³, 4×10⁻³, 3×10⁻³, 2×10⁻³, 1×10⁻³, 9×10⁻⁴, 8×10⁻⁴, 7×10⁻⁴, 6×10⁻⁴, 5×10⁻⁴, 4×10⁻⁴, 3×10⁻⁴, 2×10⁻⁴ sec⁻¹, or any value in between, at the neutral pH.

In some embodiments, the antibody or antigen-binding fragment thereof that binds to human CD98 or TfR is single-chain variable fragment (scFv) comprising the heavy chain variable region (Hv) covalently linked to the light chain variable region (Lv) via a flexible linker. The scFv can retain the specificity of the original immunoglobulin, despite removal of the constant regions and the introduction of the linker. In a scFv, the order of the domains can be either Hv-linker-Lv, or Lv-linker-Hv. The linker can be designed de novo or derived from known protein structure to provide a compatible length and conformational in bridging the variable domains of a scFv without serious steric interference. The linker can have 10 to about 25 amino acids in length. Preferably, the linker is a peptide linker spanning about 3.5 nm (35 Å) between the carboxy terminus of the variable domain and the amino terminus of the other domain without affecting the ability of the domains to fold and form an intact antigen-binding site (Huston et al., Methods in Enzymology, vol. 203, pp. 46-88, 1991, which is incorporated herein by reference in its entirety). The linker preferably comprises a hydrophilic sequence in order to avoid intercalation of the peptide within or between the variable domains throughout the protein folding (Argos, Journal of Molecular Biology, vol. 211, no. 4, pp. 943-958, 1990). For example, the linker can comprise Gly and Ser residues and/or together with the charged residues such as Glu, Thr and Lys interspersed to enhance the solubility. In some embodiments, the linker has the amino acid sequence of SEQ ID NO: 50 (GGGSGGSGGCPPCGGSGG) or SEQ ID NO: 51 (GTEGKSSGSGSESKST). Any other suitable linker can also be used in view of the present disclosure.

In some embodiments, the anti-CD98 or anti-TfR antibody or antigen-binding fragment thereof comprises a heavy chain variable region comprising HCDR1, HCDR2 and HCDR3, and a light chain variable region comprising LCDR1, LCDR2 and LCDR3, wherein:
(a) the HCDR1, HCDR2, HCDR3, LCDR1, LCDR2 and LCDR3 of the anti-CD98 antibody or antigen-binding fragment thereof have the amino acid sequences of SEQ ID NOs: 26, 27, 28 or 33, 29, 30 and 31, respectively; or
(b) the HCDR1, HCDR2, HCDR3, LCDR1, LCDR2 and LCDR3 of the anti-TfR antibody or antigen-binding fragment thereof have the amino acid sequences of SEQ ID NOs: 35, 36, 37, 38, 39 and 40, respectively.

In some embodiments, the anti-CD98 or anti-TfR antibody or antigen-binding fragment thereof is a single-chain variable fragment (scFv) comprising the heavy chain variable region covalently linked to the light chain variable region via a linker, preferably, the linker has the amino acid sequence of SEQ ID NO: 50 or 51, more preferably, the scFv comprises an amino acid sequence having at least 80%, such as at least 85%, 90%, 95% or 100%, sequence identity to the amino acid sequences of SEQ ID NO: 25, SEQ ID NO: 32, SEQ ID NO: 34, or SEQ ID NO: 41.

In a preferred embodiment, an antibody or antigen binding fragment thereof that binds to CD98, preferably CD98hc, more preferably human CD98hc, or that binds to TfR, preferably TfR1, more preferably human TfR1, does not contain a free cysteine.

An anti-CD98 or anti-TfR antibody or antigen-binding fragment thereof (such as a scFv fragment) can be produced using suitable methods in the art in view of the present disclosure. For example, a scFv fragment can be recombinantly produced by growing a recombinant host cell (such as a bacterial, yeast or mammalian cell) under suitable conditions for the production of the antibody fragment and recovering the fragment from the cell culture. Exemplary anti-CD98 or anti-TfR antibodies or antigen-binding fragments thereof are found in, e.g., U.S. 63/036,020 and U.S. 63/035,961, the disclosures of which are incorporated herein in their entireties.

A humanized anti-PHF-tau antibody or antigen-binding fragment thereof of the application can be delivered in a combined form or linked to an anti-CD98 or anti-TfR antibody or antigen binding fragment thereof, parenterally, e.g., intravenously. For example, the humanized anti-PHF-tau antibody or antigen-binding fragment thereof can be non-covalently attached to the anti-CD98 or anti-TfR antibody or antigen binding fragment thereof. The humanized anti-PHF-tau antibody or antigen-binding fragment thereof can also be covalently attached to the anti-CD98 or anti-TfR antibody or antigen binding fragment thereof to form a conjugate. In certain embodiments, the conjugation is by construction of a protein fusion (i.e., by genetic fusion of the two genes encoding a humanized anti-PHF-tau antibody or antigen-binding fragment thereof and an anti-CD98 or anti-TfR antibody or antigen binding fragment thereof and expression as a single protein). Known methods can be used to link a humanized anti-PHF-tau antibody or antigen-binding fragment thereof to a CD98 or TfR antibody or antigen binding fragment thereof in view of the present disclosure. See, for example, Wu et al., Nat Biotechnol., 23(9):1137-46, 2005; Trail et al., Cancer Immunol Immunother., 52(5):328-37, 2003; Saito et al., Adv Drug Deliv Rev., 55(2):199-215, 2003; Jones et al., Pharmaceutical Research, 24(9):1759-1771, 2007.

In some embodiments, a humanized anti-PHF-tau antibody or antigen-binding fragment thereof to be delivered to the brain and an anti-CD98 or anti-TfR antibody or antigen binding fragment thereof can be covalently linked together (or conjugated) via a non-peptide linker or a peptide linker. Examples of non-peptide linkers include, but are not limited to, polyethylene glycol, polypropylene glycol, copolymer of ethylene glycol and propylene glycol, polyoxyethylated polyol, polyvinyl alcohol, polysaccharides, dextran, polyvinyl ether, biodegradable polymer, polymerized lipid, chitins, and hyaluronic acid, or derivatives thereof, or combinations thereof. A peptide linker can be a peptide chain consisting of 1 to 50 amino acids linked by peptide bonds or a derivative thereof, whose N-terminus and C-terminus can be covalently linked to an anti-CD98 or anti-TfR antibody or an antigen binding fragment thereof.

In certain embodiments, a conjugate of the application is a multi-specific antibody comprising a first antigen binding region which binds a PHF-tau and a second antigen binding region which binds a CD98 or TfR. Techniques for making multi-specific antibodies include, but are not limited to, recombinant co-expression of two immunoglobulin heavy chain-light chain pairs having different specificities (see Milstein and Cuello, Nature 305: 537, 1983), WO 93/08829, and Traunecker et al, EMBO J. 10: 3655, 1991), and “knob-in-hole” engineering (see, e.g., U.S. Pat. No. 5,731,168). Multi-specific antibodies can also be made by engineering electrostatic steering effects (WO 2009/089004A1); cross-linking two or more antibodies or fragments (see, e.g., U.S. Pat. No. 4,676,980, and Brennan et al, Science, 229: 81, 1985); using leucine zippers (see, e.g., Kostelny et al, J. Immunol., 148(5): 1547-1553, 1992)); using “diabody” technology (see, e.g., Hollinger et al, Proc. Natl. Acad. Sci. USA, 90:6444-6448, 1993)); using single-chain Fv (sFv) dimers (see, e.g. Gruber et al, J. Immunol, 152:5368 (1994)); and preparing trispecific antibodies as described, e.g., in Tutt et al. J. Immunol. 147: 60, 1991. A multi-specific antibody of the application also encompasses antibodies having three or more functional antigen binding sites, including “Octopus antibodies” or “dual-variable domain immunoglobulins” (DVDs) (see, e.g. US 2006/0025576A1, and Wu et al. Nature Biotechnology, 25(11):1290-7, 2007). A multi-specific antibody of the application also encompasses a “Dual Acting Fab” or “DAF” comprising an antigen binding region that binds to CD98 or TfR as well as the brain antigen (e.g., Tau) (see, US 2008/0069820, for example). In one embodiment, the antibody is an antibody fragment, various such fragments being disclosed herein.

In one embodiment, a multi-specific antibody of the application is a fusion construct comprising a humanized anti-PHF-tau antibody or antigen-binding fragment thereof of the application covalently linked (or fused) to an anti-CD98 or anti-TfR antibody or antigen-binding fragment thereof. The anti-CD98 or anti-TfR antibody or antigen-binding fragment thereof can be fused to the carboxy- and/or amino-terminus of a light and/or heavy chain of the humanized anti-PHF-tau antibody or antigen-binding fragment thereof, directly or via a linker.

In one embodiment, the anti-CD98 or anti-TfR antibody or antigen-binding fragment thereof is fused to the carboxy-terminus of a light chain of the humanized anti-PHF-tau antibody or antigen-binding fragment thereof, directly or via a linker.

In another embodiment, the anti-CD98 or anti-TfR antibody or antigen-binding fragment thereof is fused to the amino-terminus of a light chain of the humanized anti-PHF-tau antibody or antigen-binding fragment thereof, directly or via a linker.

In another embodiment, the anti-CD98 or anti-TfR antibody or antigen-binding fragment thereof is fused to the carboxy-terminus of a heavy chain of the humanized anti-PHF-tau antibody or antigen-binding fragment thereof, directly or via a linker.

In another embodiment, the anti-CD98 or anti-TfR antibody or antigen-binding fragment thereof is fused to the amino-terminus of a heavy chain of the humanized anti-PHF-tau antibody or antigen-binding fragment thereof, directly or via a linker.

In a preferred embodiment, a fusion construct of the application comprises an anti-CD98 or anti-TfR antibody or antigen-binding fragment thereof, preferably an anti-huCD98hc scFv fragment or anti-huTfR1 scFv fragment, of the application covalently linked, via a linker, to the carboxy terminus of only one of the two heavy chains of a humanized anti-PHF-tau antibody or antigen-binding fragment thereof that binds to PHF-tau. Preferably, the linker has the amino acid sequence of SEQ ID NO: 52 (GGAGGA).

To facilitate the formation of a heterodimer between the two heavy chains, e.g., one with a fusion of the anti-CD98 or anti-TfR antibody or antigen-binding fragment thereof and one without, or one containing the Fc for the anti-CD98 or anti-TfR arm and one for the anti-PHF-tau arm, heterodimeric mutations can be introduced into the Fc of the two heavy chains. Examples of such Fc mutations include, but are not limited to, the Zymework mutations (see, e.g., U.S. Pat. No. 10,457,742) and the “knob in hole” mutations (see, e.g., Ridgway et al., Protein Eng., 9(7): 617-621, 1996). Other heterodimer mutations can also be used in the application. In some embodiments, a modified CH3 as described herein is used to facilitate the formation of a heterodimer between the two heavy chains.

In some embodiments, each of the two heavy chains of the isolated humanized antibody or antigen binding fragment thereof comprises one or more heterodimeric mutations, such as a modified heterodimeric CH3 domain, or one or more knob and hole mutations, as compared to a wild-type CH3 domain polypeptide. In some embodiments, the modified heterodimeric CH3 domain of the first heavy chain comprises amino acid modifications at positions T350, L351, F405, and Y407, and the modified heterodimeric CH3 domain of the second heavy chain comprises amino acid modifications at positions T350, T366, K392 and T394, wherein the amino acid modification at position T350 is T350V, T350I, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V or T366M, the amino acid modification at position K392 is K392F, K392L or K392M, and the amino acid modification at position T394 is T394W, and wherein the numbering of amino acid residues is according to the EU index as set forth in Kabat. In some embodiments, the modified heterodimeric CH3 domain of the first heavy chain comprises mutations T350V, L351Y, F405A and Y407V, and the modified heterodimeric CH3 domain of the second heavy chain comprises mutations T350V, T366L, K392L and T394W. In some embodiments, the modified heterodimeric CH3 domain of the first heavy chain comprises an amino acid modification at position T366, and the modified heterodimeric CH3 domain of the second heavy chain comprises amino acid modifications at positions T366, L368 and Y407, wherein the amino acid modification at position T366 is T366W or T366S, the amino acid modification at position L368 is L368A, and the amino acid modification at position Y407 is Y407V. In some embodiments, the modified heterodimeric CH3 domain of the second heavy chain further comprises H435R and Y436F amino acid modifications to disrupt Protein A binding and remove hole-hole homodimers during purification. In some embodiments, the modified heterodimeric CH3 domain of the first heavy chain comprises mutation T366W, and the modified heterodimeric CH3 domain of the second heavy chain comprises mutations T366S, L368A, Y407V, H435R, and Y436F.

In addition to the heterodimeric mutations, other mutations can also be introduced. In some embodiments, the Fc region of the fusion construct or bispecific antibody further comprises one or more mutations that alter (increase or diminish), preferably eliminate ADCC/CDC (such as the AAS mutations described herein), and/or one or more mutations that alter (increase or diminish), preferably increase, the binding of the fusion construct or bispecific antibody to FcRn (such as the YTE mutations described herein). In some embodiments, one or more cysteine residues in the fusion construct or bispecific antibody are substituted with other amino acids, such as serine. In some embodiments, the isolated humanized antibody or antigen-binding fragment thereof comprises one or more mutations in the Fc domain that enhance binding of the fusion to the neonatal Fc receptor (RcRn), preferably the one or more mutations enhance the binding at an acidic pH, more preferably the Fc has the M252Y/S254T/T256E (YTE) mutations, wherein the numbering of amino acid residues is according to the EU index as set forth in Kabat. In some embodiments, the isolated humanized antibody or antigen binding fragment thereof comprises one or more mutations in the Fc domain that reduce or eliminate the effector function, preferably the Fc has one or more amino acid modifications at positions L234, L235, D270, N297, E318, K320, K322, P331, and P329, such as one, two or three mutations of L234A, L235A and P331S, wherein the numbering of amino acid residues is according to the EU index as set forth in Kabat.

In certain embodiments, a fusion construct of the application comprises:
(1) a first heavy chain having an amino acid sequence that is at least 80%, such as at least 85%, 90%, 95% or 100%, identical to an amino acid sequence selected from the group consisting of SEQ ID NOs: 42-49;
(2) two light chains each independently having an amino acid sequence that is at least 80%, such as at least 85%, 90%, 95% or 100%, identical to an amino acid sequence selected from the group consisting of SEQ ID NOs: 16, 21, 24, and 60; and
(3) a second heavy chain having an amino acid sequence that is at least 80%, such as at least 85%, 90%, 95% or 100%, identical to an amino acid sequence selected from the group consisting of SEQ ID NOs: 17 and 22.

In some embodiments, a fusion construct of the application comprises a first heavy chain having the amino acid sequence of SEQ ID NO: 42, two light chains having the amino acid sequence of SEQ ID NO: 16, and a second heavy chain having the amino acid sequence of SEQ ID NO: 17. In some embodiments, a fusion construct of the application comprises a first heavy chain having the amino acid sequence of SEQ ID NO: 42, two light chains having the amino acid sequence of SEQ ID NO: 24, and a second heavy chain having the amino acid sequence of SEQ ID NO: 17. In some embodiments, a fusion construct of the application comprises a first heavy chain having the amino acid sequence of SEQ ID NO: 43, two light chains having the amino acid sequence of SEQ ID NO: 21, and a second heavy chain having the amino acid sequence of SEQ ID NO: 22. In some embodiments, a fusion construct of the application comprises a first heavy chain having the amino acid sequence of SEQ ID NO: 44, two light chains having the amino acid sequence of SEQ ID NO: 16, and a second heavy chain having the amino acid sequence of SEQ ID NO: 17. In some embodiments, a fusion construct of the application comprises a first heavy chain having the amino acid sequence of SEQ ID NO: 44, two light chains having the amino acid sequence of SEQ ID NO: 24, and a second heavy chain having the amino acid sequence of SEQ ID NO: 17. In some embodiments, a fusion construct of the application comprises a first heavy chain having the amino acid sequence of SEQ ID NO: 45, two light chains having the amino acid sequence of SEQ ID NO: 21, and a second heavy chain having the amino acid sequence of SEQ ID NO: 22. In some embodiments, a fusion construct of the application comprises a first heavy chain having the amino acid sequence of SEQ ID NO: 46, two light chains having the amino acid sequence of SEQ ID NO: 16, and a second heavy chain having the amino acid sequence of SEQ ID NO: 17. In some embodiments, a fusion construct of the application comprises a first heavy chain having the amino acid sequence of SEQ ID NO: 47, two light chains having the amino acid sequence of SEQ ID NO: 16, and a second heavy chain having the amino acid sequence of SEQ ID NO: 17. In some embodiments, a fusion construct of the application comprises a first heavy chain having the amino acid sequence of SEQ ID NO: 46, two light chains having the amino acid sequence of SEQ ID NO: 24, and a second heavy chain having the amino acid sequence of SEQ ID NO: 17. In some embodiments, a fusion construct of the application comprises a first heavy chain having the amino acid sequence of SEQ ID NO: 47, two light chains having the amino acid sequence of SEQ ID NO: 24, and a second heavy chain having the amino acid sequence of SEQ ID NO: 17. In some embodiments, a fusion construct of the application comprises a first heavy chain having the amino acid sequence of SEQ ID NO: 48, two light chains having the amino acid sequence of SEQ ID NO: 21, and a second heavy chain having the amino acid sequence of SEQ ID NO: 22. In some embodiments, a fusion construct of the application comprises a first heavy chain having the amino acid sequence of SEQ ID NO: 49, two light chains having the amino acid sequence of SEQ ID NO: 21, and a second heavy chain having the amino acid sequence of SEQ ID NO: 22. In some embodiments, a fusion construct of the application comprises a first heavy chain having the amino acid sequence of SEQ ID NO: 43, two light chains having the amino acid sequence of SEQ ID NO: 60, and a second heavy chain having the amino acid sequence of SEQ ID NO: 22. In some embodiments, a fusion construct of the application comprises a first heavy chain having the amino acid sequence of SEQ ID NO: 45, two light chains having the amino acid sequence of SEQ ID NO: 60, and a second heavy chain having the amino acid sequence of SEQ ID NO: 22. In some embodiments, a fusion construct of the application comprises a first heavy chain having the amino acid sequence of SEQ ID NO: 48, two light chains having the amino acid sequence of SEQ ID NO: 60, and a second heavy chain having the amino acid sequence of SEQ ID NO: 22. In some embodiments, a fusion construct of the application comprises a first heavy chain having the amino acid sequence of SEQ ID NO: 49, two light chains having the amino acid sequence of SEQ ID NO: 60, and a second heavy chain having the amino acid sequence of SEQ ID NO: 22.

A conjugate, such as a multi-specific antibody or fusion construct, of the application can be produced by any of a number of techniques known in the art in view of the present disclosure. For example, it can be expressed from a recombinant host cells, wherein expression vector(s) encoding the heavy and light chains of the fusion construct or multi-specific antibody is (are) transfected into a host cell by standard techniques. The host cells can be prokaryotic or eukaryotic host cells.

In an exemplary system, one or more recombinant expression vectors encoding the heterodimeric two heavy chains and the light chains of a fusion construct of the application is/are introduced into host cells by transfection or electroporation. The selected transformant host cells are cultured to allow for expression of the heavy and light chains under conditions sufficient to produce the fusion construct, and the fusion construct is recovered from the culture medium. Standard molecular biology techniques are used to prepare the recombinant expression vector, transfect the host cells, select for transformants, culture the host cells and recover the protein construct from the culture medium.

The application provides an isolated nucleic acid encoding the amino acid sequence of an anti-CD98 or anti-TfR antibody or antigen binding fragment thereof as part of a fusion construct or multispecific antibody in any of the embodiments described herein or any of the claims. The isolated nucleic acid can be part of a vector, preferably an expression vector.

In another aspect, the application relates to a host cell transformed with the vector disclosed herein. In an embodiment, the host cell is a prokaryotic cell, for example, E. coli. In another embodiment, the host cell is a eukaryotic cell, for example, a protist cell, an animal cell, a plant cell, or a fungal cell. In an embodiment, the host cell is a mammalian cell including, but not limited to, CHO, COS, NSO, SP2, PER.C6, or a fungal cell, such as Saccharomyces cerevisiae, or an insect cell, such as Sf9.

### Pharmaceutical Compositions and Methods of Treatment

Humanized anti-PHF-tau antibodies of the application or fragments thereof, conjugates thereof, or fusion constructs thereof of the application can be used to treat, reduce or prevent symptoms in patients having a neurodegenerative disease that involves pathological aggregation of tau within the brain, or a tauopathy, such as patients suffering from AD.

Thus, in another general aspect, the application relates to a pharmaceutical composition comprising an isolated humanized antibody or antigen-binding fragment thereof, a conjugate thereof, or a fusion construct thereof of the application and a pharmaceutically acceptable carrier.

In another general aspect, the application relates to a method of blocking tau seeding in a subject in need thereof, comprising administering to the subject a pharmaceutical composition of the application. A “tau seed” as used herein refers to a tau aggregate capable of nucleating or “seeding” intracellular tau aggregation when internalized by a cell, or when exposed to monomeric tau in vitro. Tau seeding activity may be assessed in cellular tau aggregation assays as described herein (see also e.g., U.S. Pat. No. 9,834,596 which is incorporated by reference in its entirety).

In another general aspect, the application relates to a method of treating or reducing symptoms of a disease, disorder or condition, such as a tauopathy, in a subject in need thereof, comprising administering to the subject a pharmaceutical composition of the application.

In another general aspect, the application relates to a method of reducing pathological tau aggregation or spreading of tauopathy in a subject in need thereof, comprising administering to the subject a pharmaceutical composition of the application.

According to embodiments of the application, the pharmaceutical composition comprises a therapeutically effective amount of the humanized anti-PHF-tau antibody or antigen-binding fragment thereof, conjugate thereof, or fusion construct thereof. As used herein with reference to humanized anti-PHF-tau antibodies or antigen-binding fragments thereof, conjugates thereof, or fusion constructs thereof, a therapeutically effective amount means an amount of the humanized anti-PHF-tau antibody or antigen-binding fragment thereof, conjugate thereof, or fusion construct thereof that results in treatment of a disease, disorder, or condition; prevents or slows the progression of the disease, disorder, or condition; or reduces or completely alleviates symptoms associated with the immune disease, disorder, or condition.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

According to particular embodiments, the disease, disorder or condition to be treated is a tauopathy. According to more particular embodiments, the disease, disorder or condition to be treated, includes, but is not limited to, familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Sträussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, or dementia pugulistica (boxing disease).

A tauopathy-related behavioral phenotype includes, but is not limited to, cognitive impairments, early personality change and disinhibition, apathy, abulia, mutism, apraxia, perseveration, stereotyped movements/behaviors, hyperorality, disorganization, inability to plan or organize sequential tasks, selfishness/callousness, antisocial traits, a lack of empathy, halting, agrammatic speech with frequent paraphasic errors but relatively preserved comprehension, impaired comprehension and word-finding deficits, slowly progressive gait instability, retropulsions, freezing, frequent falls, non-levodopa responsive axial rigidity, supranuclear gaze palsy, square wave jerks, slow vertical saccades, pseudobulbar palsy, limb apraxia, dystonia, cortical sensory loss, and tremor.

Patients amenable to treatment include, but are not limited to, asymptomatic individuals at risk of AD or other tauopathy, as well as patients presently showing symptoms. Patients amenable to treatment include individuals who have a known genetic risk of AD, such as a family history of AD or presence of genetic risk factors in the genome. Exemplary risk factors are mutations in the amyloid precursor protein (APP), especially at position 717 and positions 670 and 671 (Hardy and Swedish mutations, respectively). Other risk factors are mutations in the presenilin genes PS1 and PS2 and in ApoE4, family history of hypercholesterolemia or atherosclerosis. Individuals presently suffering from AD can be recognized from characteristic dementia by the presence of risk factors described above. In addition, a number of diagnostic tests are available to identify individuals who have AD. These include measurement of cerebrospinal fluid tau and Abeta 42 levels. Elevated tau and decreased Abeta 42 levels signify the presence of AD. Individuals suffering from AD can also be diagnosed by AD and Related Disorders Association criteria.

Anti-PHF-tau antibodies of the application are suitable both as therapeutic and prophylactic agents for treating or preventing neurodegenerative diseases that involve pathological aggregation of tau, such as AD or other tauopathies. In asymptomatic patients, treatment can begin at any age (e.g., at about 10, 15, 20, 25, 30 years). Usually, however, it is not necessary to begin treatment until a patient reaches about 40, 50, 60, or 70 years. Treatment typically entails multiple dosages over a period of time. Treatment can be monitored by assaying antibody or activated T-cell or B-cell responses to the therapeutic agent over time. If the response falls, a booster dosage can be indicated.

In prophylactic applications, pharmaceutical compositions or medicaments are administered to a patient susceptible to, or otherwise at risk of, AD in an amount sufficient to eliminate or reduce the risk, lessen the severity, or delay the outset of the disease, including biochemical, histologic and/or behavioral symptoms of the disease, its complications and intermediate pathological phenotypes presented during development of the disease. In therapeutic applications, compositions or medicaments are administered to a patient suspected of, or already suffering from, such a disease in an amount sufficient to reduce, arrest, or delay any of the symptoms of the disease (biochemical, histologic and/or behavioral). Administration of a therapeutic can reduce or eliminate mild cognitive impairment in patients that have not yet developed characteristic Alzheimer's pathology.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

In some embodiments, the application relates to a method of transporting a humanized anti-PHF-tau antibody or antigen-binding fragment thereof across the blood-brain barrier (BBB) comprising exposing an anti-CD98 or anti-TfR antibody or antigen binding fragment thereof coupled to the humanized anti-PHF-tau antibody or antigen-binding fragment thereof to the blood-brain barrier such that the anti-CD98 or anti-TfR antibody or antigen binding fragment thereof transports the humanized anti-PHF-tau antibody or antigen-binding fragment thereof coupled thereto across the blood-brain barrier. Preferably, the anti-CD98 or anti-TfR antibody or antigen binding fragment thereof, conjugate thereof, or fusion construct thereof, does not interfere with amino acid transport. The antibody specifically binds to CD98 or TfR in such a manner that it does not interfere with amino acid transport. In some embodiments, the BBB is in a mammal, preferably a primate, such as a human, more preferably a human having a neurological disorder. In one embodiment, the neurological disorder is selected from the group consisting of Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, and traumatic brain injury.

The antibodies of the application can be prepared as pharmaceutical compositions containing a therapeutically effective amount of the antibody or antibodies, conjugate, or fusion construct, as an active ingredient in a pharmaceutically acceptable carrier. The carrier can be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They can be sterilized by conventional, well-known sterilization techniques (e.g., filtration). The compositions can contain pharmaceutically acceptable auxiliary substances as required to approximate physiological conditions such as pH adjusting and buffering agents, stabilizing, thickening, lubricating and coloring agents, etc. The concentration of the antibodies of the application in such pharmaceutical formulation can vary widely, i.e., from less than about 0.5%, usually at or at least about 1% to as much as 15 or 20% by weight and will be selected primarily based on required dose, fluid volumes, viscosities, etc., according to the particular mode of administration selected.

The mode of administration for therapeutic use of the antibodies of the application can be any suitable route that delivers the agent to the host. For example, the compositions described herein can be formulated to be suitable for parenteral administration, e.g., intradermal, intramuscular, intraperitoneal, intravenous, subcutaneous, intranasal or intracranial administration, or they can be administered into the cerebrospinal fluid of the brain or spine.

The treatment can be given in a single dose schedule, or as a multiple dose schedule in which a primary course of treatment can be with 1-10 separate doses, followed by other doses given at subsequent time intervals required to maintain and or reinforce the response, for example, at 1-4 months for a second dose, and if needed, a subsequent dose(s) after several months. Examples of suitable treatment schedules include: (i) 0, 1 month and 6 months, (ii) 0, 7 days and 1 month, (iii) 0 and 1 month, (iv) 0 and 6 months, or other schedules sufficient to elicit the desired responses expected to reduce disease symptoms or reduce severity of disease.

The antibodies of the application can be lyophilized for storage and reconstituted in a suitable carrier prior to use. This technique has been shown to be effective with antibody and other protein preparations and art-known lyophilization and reconstitution techniques can be employed.

According to particular embodiments, a composition used in the treatment of a tauopathy can be used in combination with other agents that are effective for treatment of related neurodegenerative diseases. In the case of AD, antibodies of the application can be administered in combination with agents that reduce or prevent the deposition of amyloid-beta (Abeta). It is possible that PHF-tau and Abeta pathologies are synergistic. Therefore, combination therapy targeting the clearance of both PHF-tau and Abeta and Abeta-related pathologies at the same time can be more effective than targeting each individually. In the case of Parkinson's Disease and related neurodegenerative diseases, immune modulation to clear aggregated forms of the alpha-synuclein protein is also an emerging therapy. A combination therapy which targets the clearance of both tau and alpha-synuclein proteins simultaneously can be more effective than targeting either protein individually.

In another general aspect, the application relates to a method of producing a pharmaceutical composition comprising a humanized anti-PHF-tau antibody or antigen-binding fragment thereof of the application, comprising combining a humanized anti-PHF-tau antibody or antigen-binding fragment thereof with a pharmaceutically acceptable carrier to obtain the pharmaceutical composition.

In another embodiment, the application relates to the use of a humanized anti-PHF-tau antibody or antigen-binding fragment thereof, conjugate thereof, or fusion construct thereof of the application in the manufacture or preparation of a medicament. In one embodiment, the medicament is for treatment of neurological disease or disorder. In a further embodiment, the medicament is for use in a method of treating neurological disease or disorder comprising administering to an individual having neurological disease or disorder an effective amount of the medicament.

Another general aspect of the application relates to a method of inducing antibody dependent phagocytosis (ADP) without stimulating secretion of a pro-inflammatory cytokine in a subject in need thereof, comprising administering to the subject a complex comprising a humanized anti-PHF-tau antibody or antigen-binding fragment thereof coupled to, preferably covalently conjugated to, the antigen-binding fragment thereof of an anti-CD98 or anti-TfR antibody binding fragment according to an embodiment of the application, wherein the humanized anti-PHF-tau antibody or antigen-binding fragment thereof does not have effector function. For example, the humanized anti-PHF-tau antibody or antigen-binding fragment thereof can comprise one or more amino acid modifications that reduces or eliminates the effector function, such as the ADCC or CDC, such as mutations that reduce or abolish the binding to Fc gamma receptor. Such mutations can be at positions L234, L235, D270, N297, E318, K320, K322, P331, and P329, such as one, two or three mutations of L234A, L235A and P331S, wherein the numbering of amino acid residues is according to the EU index as set forth in Kabat.

In some embodiments, the method further comprises administering to the subject an effective amount of at least one additional therapeutic agent. In certain embodiments, an additional therapeutic agent is a therapeutic agent effective to treat the same or a different neurological disorder as the humanized anti-PHF-tau antibody or antigen-binding fragment thereof, conjugate thereof, or fusion construct thereof, is being employed to treat. Exemplary additional therapeutic agents include, but are not limited to: the various neurological drugs described above, cholinesterase inhibitors (such as donepezil, galantamine, rovastigmine, and tacrine), NMDA receptor antagonists (such as memantine), amyloid beta peptide aggregation inhibitors, antioxidants, γ-secretase modulators, nerve growth factor (NGF) mimics or NGF gene therapy, PPARy agonists, HMS-CoA reductase inhibitors (statins), ampakines, calcium channel blockers, GABA receptor antagonists, glycogen synthase kinase inhibitors, intravenous immunoglobulin, muscarinic receptor agonists, nicrotinic receptor modulators, active or passive amyloid beta peptide immunization, phosphodiesterase inhibitors, serotonin receptor antagonists and anti-amyloid beta peptide antibodies. In certain embodiments, the at least one additional therapeutic agent is selected for its ability to mitigate one or more side effects of the neurological drug. The additional therapeutic agent can be administered in the same or separate formulations and administered together or separately with the humanized anti-PHF-tau antibody or antigen-binding fragment thereof, conjugate thereof, or fusion construct thereof. The humanized anti-PHF-tau antibody or antigen-binding fragment thereof, conjugate thereof, or fusion construct thereof of the application can be administered prior to, simultaneously with, and/or following, the administration of the additional therapeutic agent and/or adjuvant. The humanized anti-PHF-tau antibody or antigen-binding fragment thereof, conjugate thereof, or fusion construct thereof, of the application can also be used in combination with other interventional therapies such as, but not limited to, radiation therapy, behavioral therapy, or other therapies known in the art and appropriate for the neurological disorder to be treated or prevented.

In another aspect, the application relates to an article of manufacture (such as a kit) containing materials useful for the treatment or prevention of the disorders described above. The article of manufacture comprises a container and a label or package insert on or associated with the container. Suitable containers include, for example, bottles, vials, syringes, IV solution bags, etc. The containers can be formed from a variety of materials such as glass or plastic. The container holds a composition which is by itself or combined with another composition effective for treating or preventing the condition and may have a sterile access port (for example the container may be an intravenous solution bag or a vial having a stopper pierceable by a hypodermic injection needle). At least one active agent in the composition is an antibody, antigen binding fragment thereof, conjugate thereof, or fusion construct thereof, of the application. The label or package insert indicates that the composition is used for treating the condition of choice. Moreover, the article of manufacture can include (a) a first container with a composition contained therein, wherein the composition comprises an antibody, antigen binding fragment thereof or a conjugate of the application; and (b) a second container with a composition contained therein, wherein the composition comprises a further cytotoxic or otherwise therapeutic agent. The article of manufacture in this embodiment of the application can further include a package insert indicating that the compositions can be used to treat a particular condition. Optionally, the article of manufacture can further comprise a second (or third) container comprising a pharmaceutically acceptable buffer, such as bacteriostatic water for injection (BWFI), phosphate-buffered saline, Ringer's solution and dextrose solution. It can further include other materials desirable from a commercial and user standpoint, including other buffers, diluents, filters, needles, and syringes.

### Diagnostic Methods and Kits

Humanized anti-PHF-tau antibodies of the application can be used in methods of diagnosing AD or other tauopathies in a subject.

Thus, in another general aspect, the application relates to methods of detecting the presence of PHF-tau in a subject and methods of diagnosing tauopathies in a subject by detecting the presence of PHF-tau in the subject using a humanized antibody or antigen-binding fragment thereof of the application.

In one embodiment, a humanized anti-PHF-tau antibody or antigen-binding fragment thereof, conjugate thereof, or fusion construct thereof of the application, is used to detect a neurological disorder before the onset of symptoms and/or to assess the severity or duration of the disease or disorder. The antibody, antigen binding fragment, conjugate thereof, or fusion construct thereof, permits detection and/or imaging of the neurological disorder, including imaging by radiography, tomography, or magnetic resonance imaging (MRI).

Phosphorylated tau can be detected in a biological sample from a subject (e.g., blood, serum, plasma, interstitial fluid, or cerebral spinal fluid sample) by contacting the biological sample with the diagnostic antibody reagent and detecting binding of the diagnostic antibody reagent to phosphorylated tau in the sample from the subject. Assays for carrying out the detection include well-known methods such as ELISA, immunohistochemistry, western blot, or in vivo imaging.

Diagnostic antibodies or similar reagents can be administered by intravenous injection into the body of the patient, or directly into the brain by any suitable route that delivers the agent to the host. The dosage of antibody should be within the same ranges as for treatment methods. Typically, the antibody is labeled, although in some methods, the primary antibody with affinity for phosphorylated tau is unlabeled, and a secondary labeling agent is used to bind to the primary antibody. The choice of label depends on the means of detection. For example, a fluorescent label is suitable for optical detection. Use of paramagnetic labels is suitable for tomographic detection without surgical intervention. Radioactive labels can also be detected using PET or SPECT.

Diagnosis is performed by comparing the number, size, and/or intensity of labeled PHF-tau, tau aggregates, and/or neurofibrillary tangles in a sample from the subject or in the subject, to corresponding baseline values. The baseline values can represent the mean levels in a population of healthy individuals. Baseline values can also represent previous levels determined in the same subject.

The diagnostic methods described above can also be used to monitor a subject's response to therapy by detecting the presence of phosphorylated tau in a subject before, during or after the treatment. A decrease in values relative to baseline signals a positive response to treatment. Values can also increase temporarily in biological fluids as pathological tau is being cleared from the brain.

The present application is further directed to a kit for performing the above described diagnostic and monitoring methods. Typically, such kits contain a diagnostic reagent such as the antibodies of the application, and optionally a detectable label. The diagnostic antibody itself can contain the detectable label (e.g., fluorescent molecule, biotin, etc.) which is directly detectable or detectable via a secondary reaction (e.g., reaction with streptavidin). Alternatively, a second reagent containing the detectable label cab be used, where the second reagent has binding specificity for the primary antibody. In a diagnostic kit suitable for measuring PHF-tau in a biological sample, the antibodies of the kit can be supplied pre-bound to a solid phase, such as to the wells of a microtiter dish.

### Embodiments

The application provides also the following non-limiting embodiments.

Embodiment 1 is an isolated humanized antibody or antigen-binding fragment thereof that binds to a tau protein at an epitope of the tau protein having or within the amino acid sequence of SEQ ID NO: 1, wherein the antibody or antigen-binding fragment thereof binds paired helical filament (PHF)-tau, preferably human PHF-tau, wherein the epitope of the tau protein comprises one or more of phosphorylated T427, phosphorylated S433 and phosphorylated S435 of the tau protein, but does not comprise all of phosphorylated T427, phosphorylated S433 and phosphorylated S435.

Embodiment 2 is the isolated humanized antibody or antigen-binding fragment thereof of embodiment 1 comprising immunoglobulin heavy chain complementarity determining regions (HCDRs) HCDR1, HCDR2 and HCDR3 having the polypeptide sequences of SEQ ID NOs: 4, 5 and 6, respectively; and immunoglobulin light chain complementarity determining regions (LCDRs) LCDR1, LCDR2 and LCDR3 having the polypeptide sequences of SEQ ID NOs: 7 or 14, 8 and 9, respectively; wherein the isolated humanized antibody or antigen-binding fragment thereof comprises a heavy chain variable region having a polypeptide sequence at least 90% identical to SEQ ID NO: 12 or 18, or a light chain variable region having a polypeptide sequence at least 90% identical to SEQ ID NO: 13, 19, 23 or 59.

Embodiment 2a is the isolated humanized antibody or antigen-binding fragment thereof of embodiment 2, comprising immunoglobulin heavy chain complementarity determining regions (HCDRs) HCDR1, HCDR2 and HCDR3 having the polypeptide sequences of SEQ ID NOs: 4, 5 and 6, respectively; and immunoglobulin light chain complementarity determining regions (LCDRs) LCDR1, LCDR2 and LCDR3 having the polypeptide sequences of SEQ ID NOs: 7, 8 and 9, respectively.

Embodiment 2b is the isolated humanized antibody or antigen-binding fragment thereof of embodiment 2, comprising immunoglobulin heavy chain complementarity determining regions (HCDRs) HCDR1, HCDR2 and HCDR3 having the polypeptide sequences of SEQ ID NOs: 4, 5 and 6, respectively; and immunoglobulin light chain complementarity determining regions (LCDRs) LCDR1, LCDR2 and LCDR3 having the polypeptide sequences of SEQ ID NOs: 14, 8 and 9, respectively.

Embodiment 3 is the isolated humanized antibody or antigen-binding fragment thereof of embodiment 2, comprising a heavy chain variable region having the polypeptide sequence of SEQ ID NO: 12 or 18, or a light chain variable region having the polypeptide sequence of SEQ ID NO: 13, 19, 23 or 59.

Embodiment 4 is the isolated humanized antibody or antigen-binding fragment thereof of any one of embodiments 1 to 3, comprising:
(a) a heavy chain variable region having the polypeptide sequence of SEQ ID NO: 12, and a light chain variable region having the polypeptide sequence of SEQ ID NO: 13;
(b) a heavy chain variable region having the polypeptide sequence of SEQ ID NO: 18, and a light chain variable region having the polypeptide sequence of SEQ ID NO: 19;
(c) a heavy chain variable region having the polypeptide sequence of SEQ ID NO: 12, and a light chain variable region having the polypeptide sequence of SEQ ID NO: 23; or
(d) a heavy chain variable region having the polypeptide sequence of SEQ ID NO: 18, and a light chain variable region having the polypeptide sequence of SEQ ID NO: 59.

Embodiment 6a is the isolated humanized antibody or antigen-binding fragment thereof of embodiment 6 comprising a heavy chain variable region having the polypeptide sequence of SEQ ID NO: 12, and a light chain variable region having the polypeptide sequence of SEQ ID NO: 13.

Embodiment 4b is the isolated humanized antibody or antigen-binding fragment thereof of embodiment 4 comprising a heavy chain variable region having the polypeptide sequence of SEQ ID NO: 18, and a light chain variable region having the polypeptide sequence of SEQ ID NO: 19.

Embodiment 4c is the isolated humanized antibody or antigen-binding fragment thereof of embodiment 4 comprising a heavy chain variable region having the polypeptide sequence of SEQ ID NO: 12, and a light chain variable region having the polypeptide sequence of SEQ ID NO: 23.

Embodiment 4d is the isolated humanized antibody or antigen-binding fragment thereof of embodiment 4 comprising a heavy chain variable region having the polypeptide sequence of SEQ ID NO: 18, and a light chain variable region having the polypeptide sequence of SEQ ID NO: 59.

Embodiment 5 is the isolated humanized antibody or antigen-binding fragment thereof of any one of embodiments 1 to 4, comprising:
(a) a first heavy chain having the polypeptide sequence of SEQ ID NO: 15 or 20;
(b) two light chains each independently having the polypeptide sequence of SEQ ID NO: 16, 21, 24 or 60; and
(c) a second heavy chain having the polypeptide sequence of SEQ ID NO: 17 or 22.

Embodiment 5a is the isolated humanized antibody or antigen-binding fragment thereof of embodiment 5, comprising a first heavy chain having the polypeptide sequence of SEQ ID NO: 15, two light chains each having the polypeptide sequence of SEQ ID NO: 16, and a second heavy chain having the polypeptide sequence of SEQ ID NO: 17.

Embodiment 5b is the isolated humanized antibody or antigen-binding fragment thereof of embodiment 5, comprising a first heavy chain having the polypeptide sequence of SEQ ID NO: 20, two light chains each having the polypeptide sequence of SEQ ID NO: 21, and a second heavy chain having the polypeptide sequence of SEQ ID NO: 22.

Embodiment 5c is the isolated humanized antibody or antigen-binding fragment thereof of embodiment 5, comprising a first heavy chain having the polypeptide sequence of SEQ ID NO: 15, two light chains each having the polypeptide sequence of SEQ ID NO: 24, and a second heavy chain having the polypeptide sequence of SEQ ID NO: 17.

Embodiment 5d is the isolated humanized antibody or antigen-binding fragment thereof of embodiment 5, comprising a first heavy chain having the polypeptide sequence of SEQ ID NO: 20, two light chains each having the polypeptide sequence of SEQ ID NO: 60, and a second heavy chain having the polypeptide sequence of SEQ ID NO: 22.

Embodiment 5e is the isolated humanized antibody or antigen-binding fragment thereof of any one of embodiments 1 to 4, comprising:
(a) a heavy chain having the polypeptide sequence of SEQ ID NO: 61 or 62; and
(b) a light chain having the polypeptide sequence of SEQ ID NO: 16 or 24, or 21 or 60.

Embodiment 5f is the isolated humanized antibody or antigen-binding fragment thereof of embodiment 5e, comprising a heavy chain having the polypeptide sequence of SEQ ID NO: 61, and a light chain having the polypeptide sequence of SEQ ID NO: 16 or 24.

Embodiment 5g is the isolated humanized antibody or antigen-binding fragment thereof of embodiment 5e, comprising a heavy chain having the polypeptide sequence of SEQ ID NO: 62, and a light chain having the polypeptide sequence of SEQ ID NO: 21 or 60.

Embodiment 6 is a conjugate comprising the isolated humanized antibody or antigen-binding fragment thereof of any one of embodiments 1 to 5g coupled to an anti-CD98 or anti-TfR antibody or antigen-binding fragment thereof.

Embodiment 7 is the conjugate of embodiment 6, wherein the anti-CD98 or anti-TfR antibody or antigen-binding fragment thereof binds to a CD98, preferably human CD98hc, or a TfR, preferably human TfR1, respectively, with a dissociation constant K_{D} of at least 1 nM, preferably 1-500 nM, at neutral pH and an off-rate constant k_{d} of at least 10⁻⁴ sec⁻¹, preferably 10-4 to 10⁻¹ sec⁻¹, at an acidic pH, preferably pH 5.

Embodiment 8 is the conjugate of embodiment 6 or 7, wherein the anti-CD98 or anti-TfR antibody or antigen-binding fragment thereof has an off-rate constant k_{d} of 2×10⁻² to 2×10⁻⁴ sec⁻¹, preferably 8×10⁻³ sec⁻¹ at the neutral pH.

Embodiment 9 is the conjugate of any one of embodiments 6-8, wherein the anti-CD98 or anti-TfR antibody or antigen-binding fragment thereof comprises a heavy chain variable region comprising HCDR1, HCDR2 and HCDR3, and a light chain variable region comprising LCDR1, LCDR2 and LCDR3, wherein:
(a) the HCDR1, HCDR2, HCDR3, LCDR1, LCDR2 and LCDR3 of the anti-CD98 antibody or antigen-binding fragment thereof have the amino acid sequences of SEQ ID NOs: 26, 27, 28 or 33, 29, 30 and 31, respectively; or
(b) the HCDR1, HCDR2, HCDR3, LCDR1, LCDR2 and LCDR3 of the anti-TfR antibody or antigen-binding fragment thereof have the amino acid sequences of SEQ ID NOs: 35, 36, 37, 38, 39 and 401, respectively.

Embodiment 9a is the conjugate of embodiment 9, wherein the anti-CD98 antibody or antigen-binding fragment thereof comprises a heavy chain variable region comprising HCDR1, HCDR2 and HCDR3 having the amino acid sequences of SEQ ID NOs: 26, 27 and 28, respectively, and a light chain variable region comprising LCDR1, LCDR2 and LCDR3 having the amino acid sequences of SEQ ID NOs: 29, 30 and 31, respectively.

Embodiment 9b is the conjugate of embodiment 9, wherein the anti-CD98 antibody or antigen-binding fragment thereof comprises a heavy chain variable region comprising HCDR1, HCDR2 and HCDR3 having the amino acid sequences of SEQ ID NOs: 26, 27 and 33, respectively, and a light chain variable region comprising LCDR1, LCDR2 and LCDR3 having the amino acid sequences of SEQ ID NOs: 29, 30 and 31, respectively.

Embodiment 9c is the conjugate of embodiment 9, wherein the anti-TfR antibody or antigen-binding fragment thereof comprises a heavy chain variable region comprising HCDR1, HCDR2 and HCDR3 having the amino acid sequences of SEQ ID NOs: 36, 36 and 37, respectively, and a light chain variable region comprising LCDR1, LCDR2 and LCDR3 having the amino acid sequences of SEQ ID NOs: 38, 39 and 40, respectively.

Embodiment 10 is the conjugate of any one of embodiments 6-9c, wherein the anti-CD98 or anti-TfR antibody or antigen-binding fragment thereof is a single-chain variable fragment (scFv) comprising the heavy chain variable region covalently linked to the light chain variable region via a linker, preferably, the linker has the amino acid sequence of SEQ ID NO: 50 or 51, more preferably, the scFv comprises an amino acid sequence having at least 80%, such as at least 85%, 90%, 95% or 100%, sequence identity to the amino acid sequences of SEQ ID NO: 25, SEQ ID NO: 32, SEQ ID NO: 34, or SEQ ID NO: 41.

Embodiment 10a is the conjugate of embodiment 10, wherein the anti-CD98 antibody or antigen-binding fragment thereof is a scFv comprising an amino acid sequence having at least 80%, such as at least 85%, 90%, 95% or 100%, sequence identity to the amino acid sequence of SEQ ID NO: 25.

Embodiment 10b is the conjugate of embodiment 10, wherein the anti-CD98 antibody or antigen-binding fragment thereof is a scFv comprising an amino acid sequence having at least 80%, such as at least 85%, 90%, 95% or 100%, sequence identity to the amino acid sequence of SEQ ID NO: 32.

Embodiment 10c is the conjugate of embodiment 10, wherein the anti-TfR antibody or antigen-binding fragment thereof is a scFv comprising an amino acid sequence having at least 80%, such as at least 85%, 90%, 95% or 100%, sequence identity to the amino acid sequence of SEQ ID NO: 34.

Embodiment 10d is the conjugate of embodiment 10, wherein the anti-TfR antibody or antigen-binding fragment thereof is a scFv comprising an amino acid sequence having at least 80%, such as at least 85%, 90%, 95% or 100%, sequence identity to the amino acid sequence of SEQ ID NO: 41.

Embodiment 11 is a fusion construct comprising the conjugate of any one of embodiments 6-10, wherein the anti-CD98 or anti-TfR antibody or antigen-binding fragment thereof is covalently linked to the carboxy terminus of only one of the two heavy chains of the isolated humanized antibody or antigen-binding fragment thereof via a linker, preferably wherein the linker has the amino acid sequence of SEQ ID NO: 52.

Embodiment 12 is the fusion construct of embodiment 11, wherein each of the two heavy chains of the isolated humanized antibody or antigen binding fragment thereof comprises one or more heterodimeric mutations, such as a modified heterodimeric CH3 domain, or one or more knob and hole mutations, as compared to a wild-type CH3 domain polypeptide.

Embodiment 13 is the fusion construct of embodiment 12, wherein the modified heterodimeric CH3 domain of the first heavy chain comprises amino acid modifications at positions T350, L351, F405, and Y407, and the modified heterodimeric CH3 domain of the second heavy chain comprises amino acid modifications at positions T350, T366, K392 and T394, wherein the amino acid modification at position T350 is T350V, T350I, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V or T366M, the amino acid modification at position K392 is K392F, K392L or K392M, and the amino acid modification at position T394 is T394W, and wherein the numbering of amino acid residues is according to the EU index as set forth in Kabat.

Embodiment 13a is the fusion construct of embodiment 13, wherein the modified heterodimeric CH3 domain of the first heavy chain comprises mutations T350V, L351Y, F405A and Y407V, and the modified heterodimeric CH3 domain of the second heavy chain comprises mutations T350V, T366L, K392L and T394W.

Embodiment 14 is the fusion construct of embodiment 12, wherein the modified heterodimeric CH3 domain of the first heavy chain comprises mutation T366W, and the modified heterodimeric CH3 domain of the second heavy chain comprises mutations T366S, L368A and Y407V.

Embodiment 14a is the fusion construct of embodiment 14, wherein the modified heterodimeric CH3 domain of the second heavy chain further comprises mutations H435R and Y436F.

Embodiment 15 is the fusion construct of any one of embodiments 11-14a, wherein the isolated humanized antibody or antigen-binding fragment thereof comprises one or more mutations in the Fc domain that enhance binding of the fusion to the neonatal Fc receptor (RcRn), preferably the one or more mutations enhance the binding at an acidic pH, more preferably the Fc has the M252Y/S254T/T256E (YTE) mutations, wherein the numbering of amino acid residues is according to the EU index as set forth in Kabat.

Embodiment 16 is the fusion construct of any one of embodiments 11-15, wherein the isolated humanized antibody or antigen binding fragment thereof comprises one or more mutations in the Fc domain that reduce or eliminate the effector function, preferably the Fc has one or more amino acid modifications at positions L234, L235, D270, N297, E318, K320, K322, P331, and P329, such as one, two or three mutations of L234A, L235A and P331S, wherein the numbering of amino acid residues is according to the EU index as set forth in Kabat.

Embodiment 17 is a fusion construct comprising:
(a) a first heavy chain having at least 80%, such as at least 85%, 90%, 95% or 100%, sequence identity to an amino acid sequence selected from the group consisting of SEQ ID NOs: 42-49;
(b) two light chains each independently having at least 80%, such as at least 85%, 90%, 95% or 100%, sequence identity to an amino acid sequence selected from the group consisting of SEQ ID NOs: 16, 21, 24, and 60; and
(c) a second heavy chain having at least 80%, such as at least 85%, 90%, 95% or 100%, sequence identity to an amino acid sequence selected from the group consisting of SEQ ID NOs: 17 and 22.

Embodiment 17a is the fusion construct of embodiment 17, comprising a first heavy chain having the amino acid sequence of SEQ ID NO: 42, two light chains each having the amino acid sequence of SEQ ID NO: 16, and a second heavy chain having the amino acid sequence of SEQ ID NO: 17.

Embodiment 17b is the fusion construct of embodiment 17, comprising a first heavy chain having the amino acid sequence of SEQ ID NO: 42, two light chains each having the amino acid sequence of SEQ ID NO: 24, and a second heavy chain having the amino acid sequence of SEQ ID NO: 17.

Embodiment 17c is the fusion construct of embodiment 17, comprising a first heavy chain having the amino acid sequence of SEQ ID NO: 43, two light chains each having the amino acid sequence of SEQ ID NO: 21, and a second heavy chain having the amino acid sequence of SEQ ID NO: 22.

Embodiment 17d is the fusion construct of embodiment 17, comprising a first heavy chain having the amino acid sequence of SEQ ID NO: 44, two light chains each having the amino acid sequence of SEQ ID NO: 16, and a second heavy chain having the amino acid sequence of SEQ ID NO: 17.

Embodiment 17e is the fusion construct of embodiment 17, comprising a first heavy chain having the amino acid sequence of SEQ ID NO: 44, two light chains each having the amino acid sequence of SEQ ID NO: 24, and a second heavy chain having the amino acid sequence of SEQ ID NO: 17.

Embodiment 17f is the fusion construct of embodiment 17, comprising a first heavy chain having the amino acid sequence of SEQ ID NO: 45, two light chains each having the amino acid sequence of SEQ ID NO: 21, and a second heavy chain having the amino acid sequence of SEQ ID NO: 22.

Embodiment 17g is the fusion construct of embodiment 17, comprising a first heavy chain having the amino acid sequence of SEQ ID NO: 46, two light chains each having the amino acid sequence of SEQ ID NO: 16, and a second heavy chain having the amino acid sequence of SEQ ID NO: 17.

Embodiment 17h is the fusion construct of embodiment 17, comprising a first heavy chain having the amino acid sequence of SEQ ID NO: 47, two light chains each having the amino acid sequence of SEQ ID NO: 16, and a second heavy chain having the amino acid sequence of SEQ ID NO: 17.

Embodiment 17i is the fusion construct of embodiment 17, comprising a first heavy chain having the amino acid sequence of SEQ ID NO: 46, two light chains each having the amino acid sequence of SEQ ID NO: 24, and a second heavy chain having the amino acid sequence of SEQ ID NO: 17.

Embodiment 17j is the fusion construct of embodiment 17, comprising a first heavy chain having the amino acid sequence of SEQ ID NO: 47, two light chains each having the amino acid sequence of SEQ ID NO: 24, and a second heavy chain having the amino acid sequence of SEQ ID NO: 17.

Embodiment 17k is the fusion construct of embodiment 17, comprising a first heavy chain having the amino acid sequence of SEQ ID NO: 48, two light chains each having the amino acid sequence of SEQ ID NO: 21, and a second heavy chain having the amino acid sequence of SEQ ID NO: 22.

Embodiment 17l is the fusion construct of embodiment 17, comprising a first heavy chain having the amino acid sequence of SEQ ID NO: 49, two light chains each having the amino acid sequence of SEQ ID NO: 21, and a second heavy chain having the amino acid sequence of SEQ ID NO: 22.

Embodiment 17m is the fusion construct of embodiment 17, comprising a first heavy chain having the amino acid sequence of SEQ ID NO: 43, two light chains each having the amino acid sequence of SEQ ID NO: 60, and a second heavy chain having the amino acid sequence of SEQ ID NO: 22.

Embodiment 17n is the fusion construct of embodiment 17, comprising a first heavy chain having the amino acid sequence of SEQ ID NO: 45, two light chains each having the amino acid sequence of SEQ ID NO: 60, and a second heavy chain having the amino acid sequence of SEQ ID NO: 22.

Embodiment 17o is the fusion construct of embodiment 17, comprising a first heavy chain having the amino acid sequence of SEQ ID NO: 48, two light chains each having the amino acid sequence of SEQ ID NO: 60, and a second heavy chain having the amino acid sequence of SEQ ID NO: 22.

Embodiment 17p is the fusion construct of embodiment 17, comprising a first heavy chain having the amino acid sequence of SEQ ID NO: 49, two light chains each having the amino acid sequence of SEQ ID NO: 60, and a second heavy chain having the amino acid sequence of SEQ ID NO: 22.

Embodiment 18 is an isolated nucleic acid encoding the isolated humanized antibody or antigen-binding fragment thereof of any of embodiments 1-5g, the conjugate of any one of embodiments 6-10d, or the fusion construct of any one of embodiments 11-17p.

Embodiment 19 is a vector comprising the isolated nucleic acid of embodiment 18.

Embodiment 20 is a host cell comprising the isolated nucleic acid of embodiment 18 or the vector of embodiment 19.

Embodiment 21 is a method of producing the humanized antibody or antigen-binding fragment thereof of any one of embodiments 1-5g, the conjugate of any one of embodiments 6-10, or the fusion construct of any one of embodiments 11-17p, comprising culturing a cell comprising a nucleic acid encoding the humanized antibody or antigen-binding fragment thereof, the conjugate, or the fusion construct under conditions to produce the humanized antibody or antigen-binding fragment thereof, the conjugate, or the fusion construct, and recovering the humanized antibody or antigen-binding fragment thereof, the conjugate, or the fusion construct from the cell or cell culture.

Embodiment 22 is a pharmaceutical composition comprising the isolated humanized antibody or antigen-binding fragment thereof of any one of embodiments 1-5g, the conjugate of any one of embodiments 6-10d, or the fusion construct of any one of embodiments 11-17p, and a pharmaceutically acceptable carrier.

Embodiment 23 is a method of blocking tau seeding in a subject in need thereof, comprising administering to the subject the pharmaceutical composition of embodiment 22.

Embodiment 24 is a method of inducing antibody dependent phagocytosis (ADP) without stimulating secretion of a pro-inflammatory cytokine in a subject in need thereof, comprising administering to the subject the pharmaceutical composition of embodiment 22.

Embodiment 25 is a method of treating a tauopathy in a subject in need thereof, comprising administering to the subject the pharmaceutical composition of embodiment 22.

Embodiment 26 is a method of reducing pathological tau aggregation or spreading of tauopathy in a subject in need thereof, comprising administering to the subject the pharmaceutical composition of embodiment 22.

Embodiment 27 is the method of embodiment 25 or 26, wherein the tauopathy is selected from the group consisting of familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Sträussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

Embodiment 28 is the method of any one of embodiments 23-27, wherein the pharmaceutical composition is administered intravenously.

Embodiment 29 is the method of any one of embodiments 23-28, wherein the pharmaceutical composition is delivered across the blood-brain barrier (BBB) of the subject.

Embodiment 30 is the method of any one of embodiments 23-29, wherein the administration reduces Fc-mediated effector function and/or does not induce rapid reticulocyte depletion.

Embodiment 31 is a method of detecting the presence of PHF-tau in a biological sample from a subject, comprising contacting the biological sample with the humanized antibody or antigen-binding fragment thereof of any one of embodiments 1-5g, and detecting binding of the humanized antibody or antigen-binding fragment thereof to PHF-tau in the sample from the subject.

Embodiment 32 is the method of embodiment 31, wherein the biological sample is a blood, serum, plasma, interstitial fluid, or cerebral spinal fluid sample.

Embodiment 33 is a method of producing a pharmaceutical composition comprising the humanized antibody or antigen-binding fragment thereof of any one of embodiments 1-5g, comprising combining the humanized antibody or antigen-binding fragment thereof with a pharmaceutically acceptable carrier to obtain the pharmaceutical composition.

Embodiment 34 is a humanized antibody or antigen-binding fragment thereof of any one of embodiments 1-5g or the pharmaceutical composition of embodiment 22 for use in treating a tauopathy in a subject in need thereof.

Embodiment 35 is the use of embodiment 34, wherein the tauopathy is selected from the group consisting of familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Sträussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

Embodiment 36 is a use of an isolated humanized antibody or antigen-binding fragment thereof of any one of embodiments 1 to 5g, the conjugate of any one of embodiments 6-10d, or the fusion construct of any one of embodiments 11-17p for manufacturing a medicament for treating a tauopathy in a subject in need thereof.

### EXAMPLES

The following examples of the application are to further illustrate the nature of the invention. It should be understood that the following examples do not limit the application and that the scope of the application is to be determined by the appended claims.

### Example 1—Antibody Generation, Expression and Purification

### Humanization of Anti-Tau Murine mAb PT/66: PT1B1142

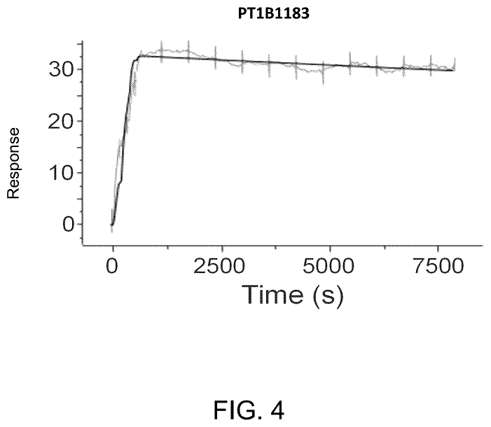

The V-regions of the light chain (LC) and the heavy chain (HC) of PT/66 (PT1B585) were humanized in the Fab format. Briefly, the V_{L} CDRs (AbM definition) from PT/66 were grafted onto the human IGKV2-24*01 germline and two positions, F41L and S28P (according to Kabat numbering system), were identified as human to mouse back mutations. The V_{H} CDRs from PT/66 were grafted onto the human IGHV1-18*01 germline and one position, A40R, was identified as human to mouse back mutation. Additionally, the V_{L} and V_{H} CDRs were grafted onto the human germlines mentioned above without human to mouse back mutations (PT1B995). One Fab variant containing all three mutations is PT1B1142 and its full human IgG version is PT1B1153. The 2^{nd} framework region in the light chain of PT1B1153 was modified to obtain another humanized antibody PT1B1183. The SPR sensorgram of PT1B1183 binding to full-length recombinant Tau protein is shown in FIG. 4.

### Binding of Humanized PT/66 Fab Variant PTIB1142 to Recombinant Tau

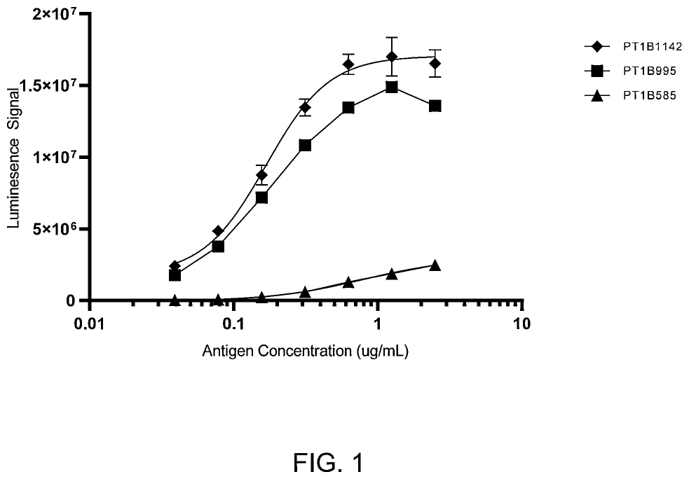

The humanized PT/66 variants were expressed in E. coli, and then screened for binding to recombinant full-length Tau protein and thermostability. Briefly, the Fab-coding sequences were cloned into a vDR001246 vector (Antibody Design Labs, San Diego, CA) having a PelB and an OmpA leader sequences for secretion of the heavy and light chains, respectively. E. coli cells (strain MC1061) were transformed with the plasmids and grown overnight at 37° C. in 2×YT microbial growth medium supplemented with 100 μg/mL Carbenicillin. Overnight cultures were used to inoculate 0.5 mL expression cultures and grown at 37° C. until OD600 ˜2.0. Protein expression was induced by addition of 1 mM IPTG and cultures were grown overnight at 30° C. After expression, cells were pelleted by centrifugation at 3,500×g for 10 min and supernatants were collected and tested directly in an ELISA assay. For ELISA, biotinylated full-length Tau protein was immobilized on the plate in concentrations ranging from 0.039 μg/mL to 2.5 μg/mL in 2-fold dilutions followed by incubation at room temperature for 45 min. Plates were blocked with 1×PBS-Tween supplemented with 3% milk. Plates were washed with 1×PBS-Tween. Supernatant was added to each plate and incubated for 45 min at room temperature. Bound Fab was detected using GoatAnitFab′2-horseradish peroxidase diluted 1:5,000 at 50 uL per well and then detected with chemiluminescence substate (Sigma cat. #11582950001). All tested Fab molecules derived from PT/66 bound the full-length Tau protein. The binding curves for PT1B1142 in comparison with PT/66 and PT1B995 are shown in FIG. 1.

### Determination of the Thermal Stability of Humanized Fab Variants

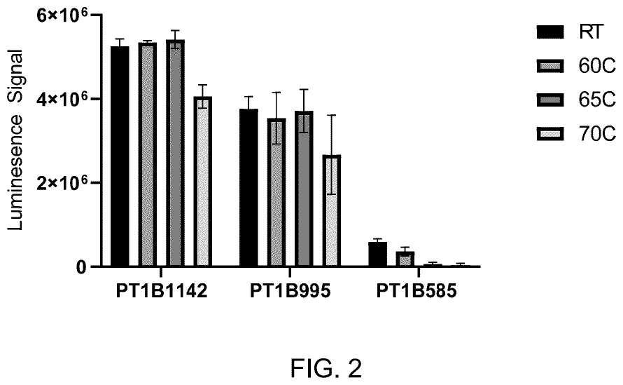

The thermal stability of the humanized PT/66 variants were determined in a heat treatment ELISA assay. The expression and ELISA assay were carried out as above (Binding). However, the expression supernatant of each variant was split into four (4) aliquots. One aliquot was kept at RT and the other three were heated to 60° C., 65° C., and 70° C., respectively, and then cooled to room temperature. ELISA binding signals (in triplicate) of all four samples were obtained. The binding signals at elevated temperatures, expressed as ratios over the RT signal, represent the thermal stability of the variants (Table 1 and FIG. 2).

TABLE 1
The binding signals at elevated temperatures, expressed as ratios over
the RT signal, represent the thermal stability of the variants.

| | | Percent Binding Retained at | | |
|---|---|---|---|---|
| Protein AA Name | RT Average | 60° C. | 65° C. | 70° C. |
| PT1B1141 | 3681333 | 98 | 107 | 84 |
| PT1B1142 | 5251067 | 102 | 103 | 77 |
| PT1B995 | 3761333 | 94 | 99 | 71 |
| PT1B585 (PT/66) | 586800 | 62 | 11 | 6 |

### Humanization of Anti-Tau Murine mAb PT/66: PT1B635 and PTIB901

In a separate attempt, PT/66 was humanized using different acceptor human frameworks. Briefly, the V_{L} CDRs from PT/66 were grafted onto the human IGKV2D-40*01 germline and the V_{H} CDRs were grafted onto the human IGHV1-18*01 germline. There were no back mutations in the light chain. However, two positions in V_{L}, M70L and T72V (Kabat numbering system), were identified as human to mouse back mutations. The humanized variant is named PT1B635. In addition, the LC from PT/66 contained a DG (positions 43-44 in CDR1) motif which presents a risk for isomerization. This risk was mitigated by making a variant containing a mutation G44L. This variant is PT1B901. PT1B635 and PT1B901 were tested for binding to recombinant tau protein by SPR.

### Antibody Expression and Purification

Research grade material was expressed at small scale (2 mL) in ExpiCHO cells by transient transfection with purified plasmid DNAs encoding PT1B635 and PT1B901. Transfected cells were incubated at 37° C. for seven days prior to harvesting the culture supernatant. The harvested supernatant was clarified by centrifugation and followed by filtration. The cell culture supernatant was purified using MabSelectSuRe Protein-A affinity chromatography and the final eluent containing monomeric protein (in Tris-Acetate buffer) was dialyzed into dPBS 7.2. The concentration of purified protein was determined by absorbance at 280 nm on a NanoDrop spectrophotometer and the quality was assessed by analytical SE-HPLC. The purity of the final was >98% and was used for follow-up studies. The following humanized antibodies were expressed and purified.

TABLE 1a
Humanized anti-Tau antibodies and their sequences.

| | | PT1B1153 | | |
|---|---|---|---|---|
| | PT1B901 | (PT1B1142) | PT1B635 | PT1B1183 |
| HCDR1-3 | SEQ ID NOs: 4, 5 & 6 | SEQ ID NOs: 4, 5 & 6 | SEQ ID NOs: 4, 5 & 6 | SEQ ID NOs: 4, 5 & 6 |
| LCDR1-3 | SEQ ID NOs: 14, 8 & 9 | SEQ ID NOs: 7, 8 & 9 | SEQ ID NOs: 7, 8 & 9 | SEQ ID NOs: 7, 8, & 9 |
| VH & VL | SEQ ID NOs: 12 & 13 | SEQ ID NOs: 18 & 19 | SEQ ID NOs: 12 & 23 | SEQ ID NOs: 18 & 59 |
| HC & LC | SEQ ID NOs: 61 & 16 | SEQ ID NOs: 62 & 21 | SEQ ID NOs: 61 & 24 | SEQ ID NOs: 62 & 60 |
| HC1, LC & | SEQ ID NOs: 15, 16 | SEQ ID NOs: 20, 21 & | SEQ ID NOs: 15, 24 & | SEQ ID NOs: 20, 60 & |
| HC2 | & 17 | 22 | 17 | 22 |

### Example 2—Binding Characterization of Anti-Tau Antibodies to Recombinant Tau by SPR

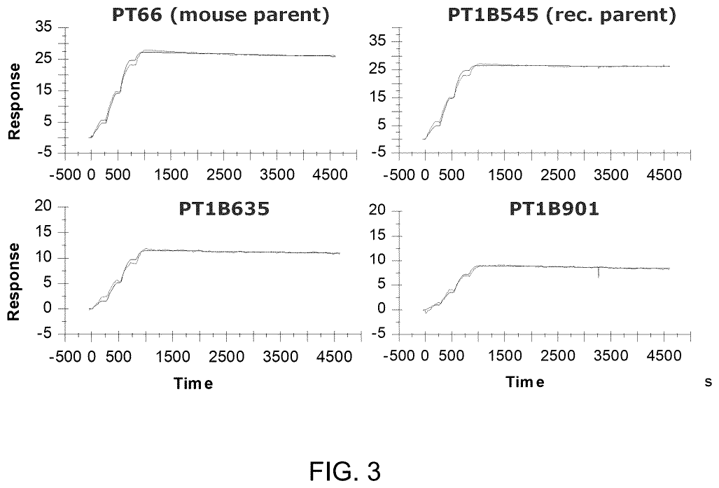

The interactions of anti-tau antibodies with human full-length recombinant tau (2N4R) were studied by SPR using a Biacore T200 at 25° C. with HBS pH 7.4 buffer, supplemented with 3 mM EDTA, and 0.05% Tween 20. Briefly, a biosensor surface was prepared by coupling a mixture of anti-mouse/anti-human IgG Fcγ-fragment specific antibody to the surface of a CM4 sensor chip using manufacturer's instructions for amine-coupling chemistry (>4000 response units (RU)). The coupling buffer was 10 mM sodium acetate, pH 4.5. The anti-tau antibodies were diluted in the running buffer and injected to obtain enough capture to enable detection of antigen binding. Capture of anti-tau mAbs was followed by injection of recombinantly expressed tau solutions ranging from 0.12 nM to 75 nM at 5-fold dilutions. The association and dissociation were monitored for 3 minutes and 60 min respectively at 50 μL/min flow rate. Regeneration of the sensor surface was performed with 0.85% H₃PO₄. The binding sensorgrams were fitted using to a 1:1 Langmuir binding model to obtain on-rates, off-rates, and affinities. PT1B635 and PT1B901 bound to full length tau protein with similar tight affinities as the parent mAbs, PT/66 or PT1B545 (FIG. 3 and Table 2 below).

TABLE 2
SPR binding kinetics and affinities of humanized
PT66 antibodies to recombinant Tau

| mAb | Description | kₒₙ (1/Ms) | k_{off} (1/s) | K_{D} (pM) |
|---|---|---|---|---|
| PT/66 | mouse parent | 4.5E+05 | <1.42E−05* | <31 |
| PT1B545 | rec. parent | 5.1E+05 | <1.42E−05* | <28 |
| PT1B635 | humanized | 3.7E+05 | <1.42E−05* | <41 |
| PT1B901 | humanized | 3.0E+05 | 2.0E−05 | 59 |
| PT1B1183 | Humanized | 1.99E+06 | 1.19E−05** | <6.18 |

k_{off} based on 1-hour limit* or 2-hour limit** and assuming 5% dissociation. Corresponding affinities are reported as “tighter than”.

### Example 3—Functional Testing by Immunodepletion

To investigate if the maximum percentage inhibition value is related to the density of epitopes on AD tau seeds or to the number of seeds that contain the PT/66 epitope, humanized variants of PT/66 will be tested for inhibition of tau seeding in an immunodepletion assay. In this assay, the mouse parent PT/66 antibody has been shown to deplete >90% of the tau seeds (see U.S. 63/166,439, the contents of which are incorporated herein in their entirety). Homogenates containing tau seeds for immunodepletion can be generated from spinal cords from 22- to 23-week old P301S transgenic animals or from cryopreserved human AD brain tissue. Biochemical analysis of the immunodepleted samples will be done by a hTau60/hTau60 and/or PT/66/PT/66 self-sandwich MSD assays which correlate very well with the cellular seeding assay (see, e.g., U.S. 63/166,439). The latter makes use of HEK cells expressing two chromophore tagged K18 tau fragments that generate a signal when in proximity by aggregation. When the cells are treated with seeds of aggregated and phosphorylated full-size tau derived from different sources, a K18 aggregate is induced that can be quantified by counting fluorescence resonance energy transfer (FRET)-positive cells using fluorescence-activated cell sorting (FACS) (Holmes et al., 2014, PNAS. 111(41): E4376-85).

AD tau seeds will be incubated with test antibody and removed from the solution with protein G beads. The depleted supernatant will be tested for residual tau seeds using PT/66/PT/66 self-sandwich MSD assays. This will confirm whether the humanized versions of PT/66 are as efficient as the parent PT/66 mAb in depleting AD tau seeds.

### Example 4—Fusion Constructs

### Fusion Construct Design

Fusion constructs containing a fusion of an antigen binding fragment (scFv) of an antibody against CD98hc or huTfR and a humanized anti-PHF-tau antibody (Tau Ab) are developed to enable the Tau Ab to penetrate the BBB, resulting in substantially higher brain concentrations compared to the Tau Ab alone.

For example, a fusion construct containing a humanized Tau Ab and a CD98hc or huTfR binding scFv appended to the C-terminus of one antibody heavy chain of the humanized Tau using a short, flexible linker. Fusion constructs are analysed for characteristics that have been previously described to enhance transcytosis (reviewed in: Goulatis and Shusta 2017): valency, binding affinity, pH dependent binding, and rapid internalization in brain endothelial cells.

Heavy and light chain variable sequences of an antibody against CD98hc or huTfR are fused in a single genetic construct as a single-chain variable fragment (scFv) using the following format:

(SEQ ID NO: 50)_Lc
Hc_GGGSGGSGGCPPCGGSGG
or
(SEQ ID NO: 51_Lc
Hc_GTEGKSSGSGSESKST

The scFv against CD98hc or huTfR is then fused to the C-terminus of one heavy chain (Hc) of a humanized Tau Ab using GGAGGA (SEQ ID NO: 52) linker. The knob and hole heterodimerization mutations in CH3 are utilized in the antibody Hc (Hc A knob: T366W; Hc B hole: T366S_L368A_Y407V) to generate the fusion construct. In addition, the RF double mutation (H435R_Y436F) in CH3 is utilized on Hc B to disrupt Protein A binding and remove hole-hole homodimers during purification. A fusion construct contains two light chains with identical amino acid sequence and two heavy chains with different amino acid sequences. Only one of the two heavy chains is fused to a scFv of a CD98 or TfR antibody and the two heavy chains also differ in their constant regions to facilitate heterodimerization between the two heavy chains. Thus, each fusion construct according to an embodiment of the application is associated with three amino acid sequences: the amino acid sequence of the first heavy chain fused to the antigen binding fragment of a CD98 or TfR antibody, the amino acid sequence of the light chain, and the amino acid sequence of the second heavy chain not fused to the antigen binding fragment of a CD98 or TfR antibody.

### Fusion Expression, Purification, and Characterization

Fusion constructs are expressed in CHO-Expi cells and purified using Protein A affinity chromatography followed by Size Exclusion chromatography or Ion-exchange chromatography.

Examples of the fusion constructs made are provided in Table 3:

TABLE 3
Tau-BBB molecules

| Name | anti-Tau mAb | BBB scFv | HC1, LC &HC2 Sequences |
|---|---|---|---|
| BBBB1910 | PT1B1183 | TFRB321 (anti-TfR) | SEQ ID NOs: 43, 60 & 22 |
| BBBB1909 | PT1B1183 | TFRB320 (anti-TfR) | SEQ ID NOs: 45, 60 & 22 |
| BBBB1626 | PT1B901 | TFRB321 (anti-TfR) | SEQ ID NOs: 42, 16 & 17 |
| BBBB1625 | PT1B635 | TFRB321 (anti-TfR) | SEQ ID NOs: 42, 24 & 17 |
| BBBB1624 | PT1B1153 | TFRB321 (anti-TfR) | SEQ ID NOs: 43, 21 & 22 |
| BBBB1622 | PT1B901 | TFRB320 (anti-TfR) | SEQ ID NOs: 44, 16 & 17 |
| BBBB1621 | PT1B635 | TFRB320 (anti-TfR) | SEQ ID NOs: 44, 24 & 17 |
| BBBB1620 | PT1B1153 | TFRB320 (anti-TfR) | SEQ ID NOs: 45, 21 & 22 |
| BBBB1548 | PT1B901 | C98B72 (anti-CD98hc) | SEQ ID NOs: 46, 16 & 17 |
| BBBB1546 | PT1B901 | C98B73 (anti-CD98hc) | SEQ ID NOs: 47, 16 & 17 |
| BBBB1545 | PT1B635 | C98B72 (anti-CD98hc) | SEQ ID NOs: 46, 24 & 17 |
| BBBB1543 | PT1B635 | C98B73 (anti-CD98hc) | SEQ ID NOs: 47, 24 & 17 |
| BBBB1508 | PT1B1153 | C98B72 (anti-CD98hc) | SEQ ID NOs: 48, 21 & 22 |
| BBBB1506 | PT1B1153 | C98B73 (anti-CD98hc) | SEQ ID NOs: 49, 21 & 22 |
| BBBB1505 | PT1B1183 | C98B72 (anti-CD98hc) | SEQ ID NOs: 48, 60 & 22 |
| BBBB1503 | PT1B1183 | C98B73 (anti-CD98hc) | SEQ ID NOs: 49, 60 & 22 |

Binding characteristics and functional testing by immunodepletion are determined for the fusion constructs using methods similar to those described in Examples 2 and 3 above.

### Internalization

Human brain endothelial cells (hCMECD3) are plated at 10,000 cells/well in Collagen-coated 384-well Cell Carrier Ultra plates (Perkin Elmer) and allowed to attach for 16 hours at 37° C. in a humidified incubator. The cells (50,000 cells) are then incubated with 200 ug/mL purified fusion construct and allowed to incubate at 37° C. for one hour. The cells are fixed, washed and incubated with a fluorescently labeled secondary antibody for one hour. The cells are then washed again and incubated with fluorescently labeled actin stain, Phalloidin, and nuclear stain, Hoeschst 33342. Cells are washed again and imaged using the ImageXpress Micro (Molecular Devices) with a 40× objective. Internalizing constructs are identified on the basis of colocalization with Phalloidin using MetaXpress 6.0.

To assess the potential of the fusion construct to promote uptake of Tau aggregates in microglia cell, human microglia derived from Induced Pluripotent Stem Cells (iPSC) are plated onto 384 well Perkin Elmer Cell Carrier Ultra plates at a dilution of 7000 cells per plate and maintained in advanced DMEM/F12 media with Glutamax+, Penicillin/Streptomycin, IL34 (100 ng/ml) and GMCSF (10 ng/ml). On the day of the assay, biotinylated tau oligomers are allowed to complex with streptavidin Alexa Flour 488 (AF488) at 15-fold molar excess. Labelled tau oligomers are then allowed to bind test constructs at approximately 2× molar excess at room temperature for 30 minutes. The Ab:tau oligomer complex is then delivered to microglia at 20 μl/well. At 2, 4 and 8 hours post incubation, cells are washed twice with phosphate buffered saline (PBS) and fixed in the presence of 4% paraformaldehyde for 15 minutes at room temperature. Following fixation, cells are once again washed twice in PBS and incubated overnight with LAMP1 primary antibody, a marker for lysosomes, at a concentration of 4 μg/ml in permeabilization buffer (0.1% saponin+1% Fish skin gelatin) at 4° C. Post incubation, cells are washed twice with PBS and stained with 1 μg/ml secondary antibody conjugated to Alexa flour 647 in permeabilization buffer for 1 hour at 4° C. Post incubation, cells are washed twice with PBS, counter stained with Hoechst DNA stain at 1 μg/ml for 10 minutes at room temperature in PBS. The cells are then washed one final time in PBS, resuspended in 20 μl of PBS per well, and imaged on the Opera Phenix confocal high content microscope. Acquired images are analyzed using Harmony and ImageJ analysis software. Approximately 500 cells per condition are scored for the presence of Tau oligomers within phagolysosomal structures, labelled with LAMP1 antibody. Fusion constructs are compared with Tau Abs for uptake into phagosomes.

While embodiments of the invention have been described in detail, and with reference to specific embodiments thereof, it will be apparent to one of ordinary skill in the art that various changes and modifications can be made therein without departing from the spirit and scope of the invention.

SEQUENCES
PHF-Tau epitope
SEQ ID NO: 1
SPQLATLADEVSASLAK
Anti-PHF-Tau antibodies
CDRs underlined
PT/66 anti-PHF-Tau monoclonal Ab
PT1B545 VH
SEQ ID NO: 2
QVQLQQPGAELVKPGASVKMSCKASGYTFTSYWITWV
KQRPGQGLEWIGDIHPGRGSTKSNEKFKSKATLTVDT
SSSTAYMQFSSLTSEDSAVYYCARRWGFDYWGQGTT
LTVSS
PT1B545 VL
SEQ ID NO: 3
DIVITQDELSNPVTSGESVSISCRSSKSLLYKDGKTYLNW
FLQRPGQSPQLLIYLMSTRASGVSDRFSGSGSGTDFTLEI
SRVKAEDVGVYYCQQLVDYPLTFGAGTKLELK
PT1B545 HCDR1
SEQ ID NO: 4
GYTFTSYWIT
PT1B545 HCDR2
SEQ ID NO: 5
DIHPGRGSTK
PT1B545 HCDR3
SEQ ID NO: 6
RWGFDY
PT1B545 LCDR1
SEQ ID NO: 7
RSSKSLLYKDGKTYLN
PT1B545 LCDR2
SEQ ID NO: 8
LMSTRAS
PT1B545 LCDR3
SEQ ID NO: 9
QQLVDYPLT
PT1B545 HC
SEQ ID NO: 10
QVQLQQPGAELVKPGASVKMSCKASGYTFTSYWITWVKQRPGQGL
EWIGDIHPGRGSTKSNEKFKSKATLTVDTSSSTAYMQFSSLTSED
SAVYYCARRWGFDYWGQGTTLTVSSAKTTAPSVYPLAPVCGDTTG
SSVTLGCLVKGYFPEPVTLTWNSGSLSSGVHTFPAVLQSDLYTLS
SSVTVTSSTWPSQSITCNVAHPASSTKVDKKIEPRGPTIKPCPPC
KCPAPNLLGGPSVFIFPPKIKDVLMISLSPIVTCVVVDVSEDDPD
VQISWFVNNVEVHTAQTQTHREDYNSTLRVVSALPIQHQDWMSGK
EFKCKVNNKDLPAPIERTISKPKGSVRAPQVYVLPPPEEEMTKKQ
VTLTCMVTDFMPEDIYVEWTNNGKTELNYKNTEPVLDSDGSYFMY
SKLRVEKKNWVERNSYSCSVVHEGLHNHHTTKSFSRTPGK
PT1B545 LC
SEQ ID NO: 11
DIVITQDELSNPVTSGESVSISCRSSKSLLYKDGKTYLNWFLQRP
GQSPQLLIYLMSTRASGVSDRFSGSGSGTDFTLEISRVKAEDVGV
YYCQQLVDYPLTFGAGTKLELKRADAAPTVSIFPPSSEQLTSGGA
SVVCFLNNFYPKDINVKWKIDGSERQNGVLNSWTDQDSKDSTYSM
SSTLTLTKDEYERHNSYTCEATHKTSTSPIVKSFNRNEC
PT1B901 humanized Ab
PT1B901 VH
SEQ ID NO: 12
QVQLVQSGAEVKKPGASVKVSCKASGYTFTSYWITWVRQ
APGQGLEWMGDIHPGRGSTKYAQKLQGRVTLTTDTSTST
AYMELRSLRSDDTAVYYCARRWGFDYWGQGTLVTVSS
PT1B901 VL
SEQ ID NO: 13
DIVITQTPLSLPVTPGEPASISCRSSKSLLYKDLKTYLNW
FLQKPGQSPQLLIYLMSTRASGVPDRFSGSGSGTDFTLKI
SRVEAEDVGVYYCQQLVDYPLTFGQGTKLEIK
PT1B901 HCDR1
SEQ ID NO: 4
GYTFTSYWIT
PT1B901 HCDR2
SEQ ID NO: 5
DIHPGRGSTK
PT1B901 HCDR3
SEQ ID NO: 6
RWGFDY
PT1B901 LCDR1
SEQ ID NO: 14
RSSKSLLYKDLKTYLN
PT1B901 LCDR2
SEQ ID NO: 8
LMSTRAS
PT1B901 LCDR3
SEQ ID NO: 9
QQLVDYPLT
PT1B901 HC1
SEQ ID NO: 15
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLWCLVKGFYPSDIAVEWESNGQPENNYKTTPPVLDSDGSFFLY
SKLTVDKSRWQQGNVFSCSVMHEALHNHYTQKSLSLSPGK
PT1B901 LC1/2
SEQ ID NO: 16
DIVITQTPLSLPVTPGEPASISCRSSKSLLYKDLKTYLNWFLQKP
GQSPQLLIYLMSTRASGVPDRFSGSGSGTDFTLKISRVEAEDVGV
YYCQQLVDYPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
PT1B901 HC2
SEQ ID NO: 17
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTS
GGTAALGCLVKDYFPEPVTVSWNSGALTSGVHTFPAVLQSSGLY
SLSSVVTVPSSSLGTQTYICNVNHKPSNTKVDKKVEPKSCDKTH
TCPPCPAPEAAGGPSVFLFPPKPKDTLYITREPEVTCVVVSVSH
EDPEVKFNWYVDGVEVHNAKTKPREEQYNSTYRVVSVLTVLHQDW
LNGKEYKCKVSNKALPAPIEKTISKAKGQPREPQVYTLPPSREEM
TKNQVSLSCAVKGFYPSDIAVEWESNGQPENNYKTTPPVLDSDGS
FFLVSKLTVDKSRWQQGNVFSCSVMHEALHNRFTQKSLS
LSPGK
PT1B901 HC
SEQ ID NO: 61
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPELLGGPSVFLFPPKPKDTLMISRTPEVTCVVVDVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLTCLVKGFYPSDIAVEWESNGQPENNYKTTPPVLDSDGSFFLY
SKLTVDKSRWQQGNVFSCSVMHEALHNHYTQKSLSLSPGK
PT1B1153 humanized Ab
PT1B1153 VH
SEQ ID NO: 18
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSS
PT1B1153 VL
SEQ ID NO: 19
DIVMTQTPLSSPVTLGQPASISCRSSKSLLYKDGKTYLNWLQQRP
GQPPRLLIYLMSTRASGVPDRFSGSGAGTDFTLKISRVEAEDVGV
YYCQQLVDYPLTFGQGTKLEIK
PT1B1153 HCDR1
SEQ ID NO: 4
GYTFTSYWIT
PT1B1153 HCDR2
SEQ ID NO: 5
DIHPGRGSTK
PT1B1153 HCDR3
SEQ ID NO: 6
RWGFDY
PT1B1153 LCDR1
SEQ ID NO: 7
RSSKSLLYKDGKTYLN
PT1B1153 LCDR2
SEQ ID NO: 8
LMSTRAS
PT1B1153 LCDR3
SEQ ID NO: 9
QQLVDYPLT
PT1B1153 HC1
SEQ ID NO: 20
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLWCLVKGFYPSDIAVEWESNGQPENNYKTTPPVLDSDGSFFLY
SKLTVDKSRWQQGNVFSCSVMHEALHNHYTQKSLSLSPGK
PT1B1153 LC1/2
SEQ ID NO: 21
DIVMTQTPLSSPVTLGQPASISCRSSKSLLYKDGKTYLNWLQQRP
GQPPRLLIYLMSTRASGVPDRFSGSGAGTDFTLKISRVEAEDVGV
YYCQQLVDYPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
PT1B1153 HC2
SEQ ID NO: 22
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLSCAVKGFYPSDIAVEWESNGQPENNYKTTPPVLDSDGSFFLV
SKLTVDKSRWQQGNVFSCSVMHEALHNRFTQKSLSLSPGK
PT1B1153 HC
SEQ ID NO: 62
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPELLGGPSVFLFPPKPKDTLMISRTPEVTCVVVDVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLTCLVKGFYPSDIAVEWESNGQPENNYKTTPPVLDSDGSFFLY
SKLTVDKSRWQQGNVFSCSVMHEALHNHYTQKSLSLSPGK
PT1B635 humanized Ab
PT1B635 VH
SEQ ID NO: 12
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSS
PT1B635 VL
SEQ ID NO: 23
DIVITQTPLSLPVTPGEPASISCRSSKSLLYKDGKTYLNWFLQKP
GQSPQLLIYLMSTRASGVPDRFSGSGSGTDFTLKISRVEAEDVGV
YYCQQLVDYPLTFGQGTKLEIK
PT1B635 HCDR1
SEQ ID NO: 4
GYTFTSYWIT
PT1B635 HCDR2
SEQ ID NO: 5
DIHPGRGSTK
PT1B635 HCDR3
SEQ ID NO: 6
RWGFDY
PT1B635 LCDR1
SEQ ID NO: 7
RSSKSLLYKDGKTYLN
PT1B635 LCDR2
SEQ ID NO: 8
LMSTRAS
PT1B635 LCDR3
SEQ ID NO: 9
QQLVDYPLT
PT1B635 HC1
SEQ ID NO: 15
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLWCLVKGFYPSDIAVEWESNGQPENNYKTTPPVLDSDGSFFLY
SKLTVDKSRWQQGNVFSCSVMHEALHNHYTQKSLSLSPGK
PT1B635 LC1/2
SEQ ID NO: 24
DIVITQTPLSLPVTPGEPASISCRSSKSLLYKDGKTYLNWFLQKP
GQSPQLLIYLMSTRASGVPDRFSGSGSGTDFTLKISRVEAEDVGV
YYCQQLVDYPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
PT1B635 HC2
SEQ ID NO: 17
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLSCAVKGFYPSDIAVEWESNGQPENNYKTTPPVLDSDGSFFLV
SKLTVDKSRWQQGNVFSCSVMHEALHNRFTQKSLSLSPGK
PT1B635 HC
SEQ ID NO: 61
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTC
PPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PAPIEKTISKAKGQPREPQVYTLPPSREEMTKNQVSLTCLVKGFY
PSDIAVEWESNGQPENNYKTTPPVLDSDGSFFLYSKLTVDKSRWQ
QGNVFSCSVMHEALHNHYTQKSLSLSPGK
Anti-CD98 and anti-TfR scFv sequences
CDRs underlined; scFv linker in italics
C98B72 (anti-CD98hc scFv)
>TPP000231088|C98B72|mature_scFv| C98B72
(anti-CD98hc)
scFv-HL [16-aa modified Bird linker]
SEQ ID NO: 25
QVQLVESGGGVVQPGRSLRLSCAASGFTESSYGMHWVRQAPGKGL
EWVAIISYDGSNKHYADSVKGRFTISRDNSKNTLYLQMNSLRAED
TAVYYCARAPSSFYFDYWGQGTLVTVSSGTEGKSSGSGSESKSTD
IVMTQSPDSLAVSLGERATINCKSSQSVLESSNNKNYLAWYQQKP
GQPPKLLIYWASTRESGVPDRFSGSGSGTDFTLTISSLQAEDVAV
YYCQQYYSTPPTFGQGTKVVIK
C98B72 HCDR1
SEQ ID NO: 26
GFTFSSYGMH
C98B72 HCDR2
SEQ ID NO: 27
IISYDGSNKH
C98B72 HCDR3
SEQ ID NO: 28
APSSFYFDY
C98B72 LCDR1
SEQ ID NO: 29
KSSQSVLFSSNNKNYLA
C98B72 LCDR2
SEQ ID NO: 30
WASTRES
C98B72 LCDR3
SEQ ID NO: 31
QQYYSTPPT
C98B73 (anti-CD98hc scFv)
>TPP000231089|C98B73|mature_scFv| C98B73
(anti-CD98hc)
scFv-HL [16-aa modified Bird linker]
SEQ ID NO: 32
QVQLVESGGGVVQPGRSLRLSCAASGFTESSYGMHWVRQAPGKGL
EWVAIISYDGSNKHYADSVKGRFTISRDNSKNTLYLQMNSLRAED
TAVYYCARAPSHFYFDYWGQGTLVTVSSGTEGKSSGSGSESKSTD
IVMTQSPDSLAVSLGERATINCKSSQSVLESSNNKNYLAWYQQKP
GQPPKLLIYWASTRESGVPDRFSGSGSGTDFTLTISSLQAEDVAV
YYCQQYYSTPPTFGQGTKVVIK
C98B73 HCDR1
SEQ ID NO: 26
GFTFSSYGMH
C98B73 HCDR2
SEQ ID NO: 27
IISYDGSNKH
C98B73 HCDR3
SEQ ID NO: 33
APSHFYFDY
C98B73 LCDR1
SEQ ID NO: 29
KSSQSVLFSSNNKNYLA
C98B73 LCDR2
SEQ ID NO: 30
WASTRES
C98B73 LCDR3
SEQ ID NO: 31
QQYYSTPPT
TFRB321 (anti-TfR scFv)
>TPP000231087I|TFRB321|mature_scFv| TFRB321
(anti-TfR) spFv-HL [stapled linker]
SEQ ID NO: 34
EVQLLESGGGLVQPGGSLRLSCADSGFTESSYAMNWVRQAPGCGL
EWVSGISGSGGHTYYADSVKGRFTVSRDNSKNTLYLQMNSLRAED
TAVYYCAREGYDSSGYNPFDYWGQGTQVTVSSGGGSGGSGGCPPC
GGSGGSYELTQPPSVSVSPGQTASITCSGDKLGDKYASWYQQKPG
QSPVLVIYQDSKRPSGIPERFSGSNSGNTATLTISGTQAMDEADY
YCQAWDSSTVVFGCGTKLTVL
TFRB321 HCDR1
SEQ ID NO: 35
GFTFSSYAMN
TFRB321 HCDR2
SEQ ID NO: 36
GISGSGGHTY
TFRB321 HCDR3
SEQ ID NO: 37
EGYDSSGYNPFDY
TFRB321 LCDR1
SEQ ID NO: 38
SGDKLGDKYAS
TFRB321 LCDR2
SEQ ID NO: 39
QDSKRPS
TFRB321 LCDR3
SEQ ID NO: 40
QAWDSSTVV
TFRB320 (anti-TfR scFv)
>TPP000231086ITFRB320|mature_scFv| TFRB320
(anti-TfR)
scFv-HL [16-aa modified Bird linker]
SEQ ID NO: 41
EVQLLESGGGLVQPGGSLRLSCADSGFTFSSYAMNWVRQAPGKGL
EWVSGISGSGGHTYYADSVKGRFTVSRDNSKNTLYLQMNSLRAED
TAVYYCAREGYDSSGYNPFDYWGQGTQVTVSSGTEGKSSGSGSES
KSTSYELTQPPSVSVSPGQTASITCSGDKLGDKYASWYQQKPGQS
PVLVIYQDSKRPSGIPERFSGSNSGNTATLTISGTQAMDEADYYC
QAWDSSTVVFGGGTKLTVL
TFRB320 HCDR1
SEQ ID NO: 35
GFTFSSYAMN
TFRB320 HCDR2
SEQ ID NO: 36
GISGSGGHTY
TFRB320 HCDR3
SEQ ID NO: 37
EGYDSSGYNPFDY
TFRB320 LCDR1
SEQ ID NO: 38
SGDKLGDKYAS
TFRB320 LCDR2
SEQ ID NO: 39
QDSKRPS
TFRB320 LCDR3
SEQ ID NO: 40
QAWDSSTVV
Anti-Tau-BBB conjugates
ScFV underlined; Hc_scFv tether
linker is in italics
BBBB1910
>BBBB1910|heavy_chain_1|
SEQ ID NO: 43
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLWCLVKGFYPSDIAVEWESNGQPENNYKTTPPVLDSDGSFFLY
SKLTVDKSRWQQGNVFSCSVMHEALHNHYTQKSLSLSPGKGGAGG
AEVQLLESGGGLVQPGGSLRLSCADSGFTESSYAMNWVRQAPGCG
LEWVSGISGSGGHTYYADSVKGRFTVSRDNSKNTLYLQMNSLRAE
DTAVYYCAREGYDSSGYNPFDYWGQGTQVTVSSGGGSGGSGGCPP
CGGSGGSYELTQPPSVSVSPGQTASITCSGDKLGDKYASWYQQKP
GQSPVLVIYQDSKRPSGIPERFSGSNSGNTATLTISGTQAMDEAD
YYCQAWDSSTVVFGCGTKLTVL
>BBBB1910|light_chain_1/2|
SEQ ID NO: 60
DIVMTQTPLSSPVTLGQPASISCRSSKSLLYKDGKTYLNWFQQRP
GQSPRLLIYLMSTRASGVPDRFSGSGAGTDFTLKISRVEAEDVGV
YYCQQLVDYPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
>BBBB1910|heavy_chain_2|
SEQ ID NO: 22
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLSCAVKGFYPSDIAVEWESNGQPENNYKTTPPVLDSDGSFFLV
SKLTVDKSRWQQGNVFSCSVMHEALHNRFTQKSLSLSPGK
BBBB1909
>BBBB1909|heavy_chain_1|
SEQ ID NO: 45
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLWCLVKGFYPSDIAVEWESNGQPENNYKTTPPVLDSDGSFFLY
SKLTVDKSRWQQGNVFSCSVMHEALHNHYTQKSLSLSPGKGGAGG
AEVQLLESGGGLVQPGGSLRLSCADSGFTFSSYAMNWVRQAPGKG
LEWVSGISGSGGHTYYADSVKGRFTVSRDNSKNTLYLQMNSLRAE
DTAVYYCAREGYDSSGYNPFDYWGQGTQVTVSSGTEGKSSGSGSE
SKSTSYELTQPPSVSVSPGQTASITCSGDKLGDKYASWYQQKPGQ
SPVLVIYQDSKRPSGIPERFSGSNSGNTATLTISGTQAMDEADYY
CQAWDSSTVVFGGGTKLTVL
>BBBB1909|light_chain_1/2|
SEQ ID NO: 60
DIVMTQTPLSSPVTLGQPASISCRSSKSLLYKDGKTYLNWFQQRP
GQSPRLLIYLMSTRASGVPDRFSGSGAGTDFTLKISRVEAEDVGV
YYCQQLVDYPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
>BBBB1909|heavy_chain_2|
SEQ ID NO: 22
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLSCAVKGFYPSDIAVEWESNGQPENNYKTTPPVLDSDGSFFLV
SKLTVDKSRWQQGNVFSCSVMHEALHNRFTQKSLSLSPGK
BBBB1626
>BBBB1626|heavy_chain_1|
SEQ ID NO: 42
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLWCLVKGFYPSDIAVEWESNGQPENNYKTTPPVLDSDGSFFLY
SKLTVDKSRWQQGNVFSCSVMHEALHNHYTQKSLSLSPGKGGAGG
AEVQLLESGGGLVQPGGSLRLSCADSGFTESSYAMNWVRQAPGCG
LEWVSGISGSGGHTYYADSVKGRFTVSRDNSKNTLYLQMNSLRAE
DTAVYYCAREGYDSSGYNPFDYWGQGTQVTVSSGGGSGGSGGCPP
CGGSGGSYELTQPPSVSVSPGQTASITCSGDKLGDKYASWYQQKP
GQSPVLVIYQDSKRPSGIPERFSGSNSGNTATLTISGTQAMDEAD
YYCQAWDSSTVVFGCGTKLTVL
>BBBB1626|light_chain_1/2|
SEQ ID NO: 16
DIVITQTPLSLPVTPGEPASISCRSSKSLLYKDLKTYLNWFLQKP
GQSPQLLIYLMSTRASGVPDRFSGSGSGTDFTLKISRVEAEDVGV
YYCQQLVDYPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
>BBBB1626|heavy_chain_2|
SEQ ID NO: 17
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLSCAVKGFYPSDIAVEWESNGQPENNYKTTPPVLDSDGSFFLV
SKLTVDKSRWQQGNVFSCSVMHEALHNRFTQKSLSLSPGK
BBBB1625
>BBBB1625|heavy_chain_1|
SEQ ID NO: 42
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLWCLVKGFYPSDIAVEWESNGQPENNYKTTPPVLDSDGSFFLY
SKLTVDKSRWQQGNVFSCSVMHEALHNHYTQKSLSLSPGKGGAGG
AEVQLLESGGGLVQPGGSLRLSCADSGFTFSSYAMNWVRQAPGCG
LEWVSGISGSGGHTYYADSVKGRFTVSRDNSKNTLYLQMNSLRAE
DTAVYYCAREGYDSSGYNPFDYWGQGTQVTVSSGGGSGGSGGCPP
CGGSGGSYELTQPPSVSVSPGQTASITCSGDKLGDKYASWYQQKP
GQSPVLVIYQDSKRPSGIPERFSGSNSGNTATLTISGTQAMDEAD
YYCQAWDSSTVVFGCGTKLTVL
>BBBB1625|light_chain_1/2|
SEQ ID NO: 24
DIVITQTPLSLPVTPGEPASISCRSSKSLLYKDGKTYLNWFLQKP
GQSPQLLIYLMSTRASGVPDRFSGSGSGTDFTLKISRVEAEDVGV
YYCQQLVDYPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
>BBBB1625|heavy_chain_2|
SEQ ID NO: 17
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLSCAVKGFYPSDIAVEWESNGQPENNYKTTPPVLDSDGSFFLV
SKLTVDKSRWQQGNVFSCSVMHEALHNRFTQKSLSLSPGK
BBBB1624
>BBBB1624|heavy_chain_1|
SEQ ID NO: 43
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLWCLVKGFYPSDIAVEWESNGQPENNYKTTPPVLDSDGSFFLY
SKLTVDKSRWQQGNVFSCSVMHEALHNHYTQKSLSLSPGKGGAGG
AEVQLLESGGGLVQPGGSLRLSCADSGFTFSSYAMNWVRQAPGCG
LEWVSGISGSGGHTYYADSVKGRFTVSRDNSKNTLYLQMNSLRAE
DTAVYYCAREGYDSSGYNPFDYWGQGTQVTVSSGGGSGGSGGCPP
CGGSGGSYELTQPPSVSVSPGQTASITCSGDKLGDKYASWYQQKP
GQSPVLVIYQDSKRPSGIPERFSGSNSGNTATLTISGTQAMDEAD
YYCQAWDSSTVVFGCGTKLTVL
>BBBB1624|light_chain_1/2|
SEQ ID NO: 21
DIVMTQTPLSSPVTLGQPASISCRSSKSLLYKDGKTYLNWLQQRP
GQPPRLLIYLMSTRASGVPDRFSGSGAGTDFTLKISRVEAEDVGV
YYCQQLVDYPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
>BBBB1624|heavy_chain_2|
SEQ ID NO: 22
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLSCAVKGFYPSDIAVEWESNGQPENNYKTTPPVLDSDGSFFLV
SKLTVDKSRWQQGNVFSCSVMHEALHNRFTQKSLSLSPGK
BBBB1622
>BBBB1622|heavy_chain_1|
SEQ ID NO: 44
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLWCLVKGFYPSDIAVEWESNGQPENNYKTTPPVLDSDGSFFLY
SKLTVDKSRWQQGNVFSCSVMHEALHNHYTQKSLSLSPGKGGAGG
AEVQLLESGGGLVQPGGSLRLSCADSGFTESSYAMNWVRQAPGKG
LEWVSGISGSGGHTYYADSVKGRFTVSRDNSKNTLYLQMNSLRAE
DTAVYYCAREGYDSSGYNPFDYWGQGTQVTVSSGTEGKSSGSGSE
SKSTSYELTQPPSVSVSPGQTASITCSGDKLGDKYASWYQQKPGQ
SPVLVIYQDSKRPSGIPERFSGSNSGNTATLTISGTQAMDEADYY
CQAWDSSTVVFGGGTKLTVL
>BBBB1622|light_chain_1/2|
SEQ ID NO: 16
DIVITQTPLSLPVTPGEPASISCRSSKSLLYKDLKTYLNWFLQKP
GQSPQLLIYLMSTRASGVPDRFSGSGSGTDFTLKISRVEAEDVGV
YYCQQLVDYPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
>BBBB1622|heavy_chain_2|
SEQ ID NO: 17
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLSCAVKGFYPSDIAVEWESNGQPENNYKTTPPVLDSDGSFFLV
SKLTVDKSRWQQGNVFSCSVMHEALHNRFTQKSLSLSPGK
BBBB1621
>BBBB1621|heavy_chain_1|
SEQ ID NO: 44
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLWCLVKGFYPSDIAVEWESNGQPENNYKTTPPVLDSDGSFFLY
SKLTVDKSRWQQGNVFSCSVMHEALHNHYTQKSLSLSPGKGGAGG
AEVQLLESGGGLVQPGGSLRLSCADSGFTESSYAMNWVRQAPGKG
LEWVSGISGSGGHTYYADSVKGRFTVSRDNSKNTLYLQMNSLRAE
DTAVYYCAREGYDSSGYNPFDYWGQGTQVTVSSGTEGKSSGSGSE
SKSTSYELTQPPSVSVSPGQTASITCSGDKLGDKYASWYQQKPGQ
SPVLVIYQDSKRPSGIPERFSGSNSGNTATLTISGTQAMDEADYY
CQAWDSSTVVFGGGTKLTVL
>BBBB1621|light_chain_1/2|
SEQ ID NO: 24
DIVITQTPLSLPVTPGEPASISCRSSKSLLYKDGKTYLNWFLQKP
GQSPQLLIYLMSTRASGVPDRFSGSGSGTDFTLKISRVEAEDVGV
YYCQQLVDYPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
>BBBB1621|heavy_chain_2|
SEQ ID NO: 17
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLSCAVKGFYPSDIAVEWESNGQPENNYKTTPPVLDSDGSFFLV
SKLTVDKSRWQQGNVFSCSVMHEALHNRFTQKSLSLSPGK
BBBB1620
>BBBB1620|heavy_chain_1|
SEQ ID NO: 45
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLWCLVKGFYPSDIAVEWESNGQPENNYKTTPPVLDSDGSFFLY
SKLTVDKSRWQQGNVFSCSVMHEALHNHYTQKSLSLSPGKGGAGG
AEVQLLESGGGLVQPGGSLRLSCADSGFTFSSYAMNWVRQAPGKG
LEWVSGISGSGGHTYYADSVKGRFTVSRDNSKNTLYLQMNSLRAE
DTAVYYCAREGYDSSGYNPFDYWGQGTQVTVSSGTEGKSSGSGSE
SKSTSYELTQPPSVSVSPGQTASITCSGDKLGDKYASWYQQKPGQ
SPVLVIYQDSKRPSGIPERFSGSNSGNTATLTISGTQAMDEADYY
CQAWDSSTVVFGGGTKLTVL
>BBBB1620|light_chain_1/2|
SEQ ID NO: 21
DIVMTQTPLSSPVTLGQPASISCRSSKSLLYKDGKTYLNWLQQRP
GQPPRLLIYLMSTRASGVPDRFSGSGAGTDFTLKISRVEAEDVGV
YYCQQLVDYPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
>BBBB1620|heavy_chain_2|
SEQ ID NO: 22
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLSCAVKGFYPSDIAVEWESNGQPENNYKTTPPVLDSDGSFFLV
SKLTVDKSRWQQGNVFSCSVMHEALHNRFTQKSLSLSPGKBBBB1548
>BBBB1548|heavy_chain_1|
SEQ ID NO: 46
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLWCLVKGFYPSDIAVEWESNGQPENNYKTTPPVLDSDGSFFLY
SKLTVDKSRWQQGNVFSCSVMHEALHNHYTQKSLSLSPGKGGAGG
AQVQLVESGGGVVQPGRSLRLSCAASGFTESSYGMHWVRQAPGKG
LEWVAIISYDGSNKHYADSVKGRFTISRDNSKNTLYLQMNSLRAE
DTAVYYCARAPSSFYFDYWGQGTLVTVSSGTEGKSSGSGSESKST
DIVMTQSPDSLAVSLGERATINCKSSQSVLFSSNNKNYLAWYQQK
PGQPPKLLIYWASTRESGVPDRESGSGSGTDFTLTISSLQAEDVA
VYYCQQYYSTPPTFGQGTKVVIK
>BBBB1548|light_chain_1/2|
SEQ ID NO: 16
DIVITQTPLSLPVTPGEPASISCRSSKSLLYKDLKTYLNWFLQKP
GQSPQLLIYLMSTRASGVPDRFSGSGSGTDFTLKISRVEAEDVGV
YYCQQLVDYPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
>BBBB1548|heavy_chain_2|
SEQ ID NO: 17
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLSCAVKGFYPSDIAVEWESNGQPENNYKTTPPVLDSDGSFFLV
SKLTVDKSRWQQGNVFSCSVMHEALHNRFTQKSLSLSPGK
BBBB1546
>BBBB1546|heavy_chain_1|
SEQ ID NO: 47
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLWCLVKGFYPSDIAVEWESNGQPENNYKTTPPVLDSDGSFFLY
SKLTVDKSRWQQGNVFSCSVMHEALHNHYTQKSLSLSPGKGGAGG
AQVQLVESGGGVVQPGRSLRLSCAASGFTESSYGMHWVRQAPGKG
LEWVAIISYDGSNKHYADSVKGRFTISRDNSKNTLYLQMNSLRAE
DTAVYYCARAPSHFYFDYWGQGTLVTVSSGTEGKSSGSGSESKST
DIVMTQSPDSLAVSLGERATINCKSSQSVLFSSNNKNYLAWYQQK
PGQPPKLLIYWASTRESGVPDRESGSGSGTDFTLTISSLQAEDVA
VYYCQQYYSTPPTFGQGTKVVIK
>BBBB1546|light_chain_1/2|
SEQ ID NO: 16
DIVITQTPLSLPVTPGEPASISCRSSKSLLYKDLKTYLNWFLQKP
GQSPQLLIYLMSTRASGVPDRFSGSGSGTDFTLKISRVEAEDVGV
YYCQQLVDYPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
>BBBB1546|heavy_chain_2|
SEQ ID NO: 17
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLSCAVKGFYPSDIAVEWESNGQPENNYKTTPPVLDSDGSFFLV
SKLTVDKSRWQQGNVFSCSVMHEALHNRFTQKSLSLSPGK
BBBB1545
>BBBB1545|heavy_chain_1|
SEQ ID NO: 46
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLWCLVKGFYPSDIAVEWESNGQPENNYKTTPPVLDSDGSFFLY
SKLTVDKSRWQQGNVFSCSVMHEALHNHYTQKSLSLSPGKGGAGG
AQVQLVESGGGVVQPGRSLRLSCAASGFTFSSYGMHWVRQAPGKG
LEWVAIISYDGSNKHYADSVKGRFTISRDNSKNTLYLQMNSLRAE
DTAVYYCARAPSSFYFDYWGQGTLVTVSSGTEGKSSGSGSESKST
DIVMTQSPDSLAVSLGERATINCKSSQSVLFSSNNKNYLAWYQQK
PGQPPKLLIYWASTRESGVPDRESGSGSGTDFTLTISSLQAEDVA
VYYCQQYYSTPPTFGQGTKVVIK
>BBBB1545|light_chain_1/2|
SEQ ID NO: 24
DIVITQTPLSLPVTPGEPASISCRSSKSLLYKDGKTYLNWFLQKP
GQSPQLLIYLMSTRASGVPDRFSGSGSGTDFTLKISRVEAEDVGV
YYCQQLVDYPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
>BBBB1545|heavy_chain_21
SEQ ID NO: 17
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLSCAVKGFYPSDIAVEWESNGQPENNYKTTPPVLDSDGSFFLV
SKLTVDKSRWQQGNVFSCSVMHEALHNRFTQKSLSLSPGK
BBBB1543
>BBBB1543|heavy_chain_1|
SEQ ID NO: 47
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLWCLVKGFYPSDIAVEWESNGQPENNYKTTPPVLDSDGSFFLY
SKLTVDKSRWQQGNVFSCSVMHEALHNHYTQKSLSLSPGKGGAGG
AQVQLVESGGGVVQPGRSLRLSCAASGFTFSSYGMHWVRQAPGKG
LEWVAIISYDGSNKHYADSVKGRFTISRDNSKNTLYLQMNSLRAE
DTAVYYCARAPSHFYFDYWGQGTLVTVSSGTEGKSSGSGSESKST
DIVMTQSPDSLAVSLGERATINCKSSQSVLESSNNKNYLAWYQQK
PGQPPKLLIYWASTRESGVPDRESGSGSGTDFTLTISSLQAEDVA
VYYCQQYYSTPPTFGQGTKVVIK
>BBBB1543|light_chain_1/2|
SEQ ID NO: 24
DIVITQTPLSLPVTPGEPASISCRSSKSLLYKDGKTYLNWFLQKP
GQSPQLLIYLMSTRASGVPDRFSGSGSGTDFTLKISRVEAEDVGV
YYCQQLVDYPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
>BBBB1543|heavy_chain_2|
SEQ ID NO: 17
QVQLVQSGAEVKKPGASVKVSCKASGYTFTSYWITWVRQAPGQGL
EWMGDIHPGRGSTKYAQKLQGRVTLTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLSCAVKGFYPSDIAVEWESNGQPENNYKTTPPVLDSDGSFFLV
SKLTVDKSRWQQGNVFSCSVMHEALHNRFTQKSLSLSPGKBBBB1508
>BBBB1508|heavy_chain_1|
SEQ ID NO: 48
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLWCLVKGFYPSDIAVEWESNGQPENNYKTTPPVLDSDGSFFLY
SKLTVDKSRWQQGNVFSCSVMHEALHNHYTQKSLSLSPGKGGAGG
AQVQLVESGGGVVQPGRSLRLSCAASGFTFSSYGMHWVRQAPGKG
LEWVAIISYDGSNKHYADSVKGRFTISRDNSKNTLYLQMNSLRAE
DTAVYYCARAPSSFYFDYWGQGTLVTVSSGTEGKSSGSGSESKST
DIVMTQSPDSLAVSLGERATINCKSSQSVLFSSNNKNYLAWYQQK
PGQPPKLLIYWASTRESGVPDRFSGSGSGTDFTLTISSLQAEDVA
VYYCQQYYSTPPTFGQGTKVVIK
>BBBB1508|light_chain_1/2|
SEQ ID NO: 21
DIVMTQTPLSSPVTLGQPASISCRSSKSLLYKDGKTYLNWLQQRP
GQPPRLLIYLMSTRASGVPDRFSGSGAGTDFTLKISRVEAEDVGV
YYCQQLVDYPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
>BBBB1508|heavy_chain_2|
SEQ ID NO: 22
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLSCAVKGFYPSDIAVEWESNGQPENNYKTTPPVLDSDGSFFLV
SKLTVDKSRWQQGNVFSCSVMHEALHNRFTQKSLSLSPGK
BBBB1506
>BBBB1506|heavy_chain_1|
SEQ ID NO: 49
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLWCLVKGFYPSDIAVEWESNGQPENNYKTTPPVLDSDGSFFLY
SKLTVDKSRWQQGNVFSCSVMHEALHNHYTQKSLSLSPGKGGAGG
AQVQLVESGGGVVQPGRSLRLSCAASGFTFSSYGMHWVRQAPGKG
LEWVAIISYDGSNKHYADSVKGRFTISRDNSKNTLYLQMNSLRAE
DTAVYYCARAPSHFYFDYWGQGTLVTVSSGTEGKSSGSGSESKST
DIVMTQSPDSLAVSLGERATINCKSSQSVLESSNNKNYLAWYQQK
PGQPPKLLIYWASTRESGVPDRESGSGSGTDFTLTISSLQAEDVA
VYYCQQYYSTPPTFGQGTKVVIK
>BBBB1506|light_chain_1/2|
SEQ ID NO: 21
DIVMTQTPLSSPVTLGQPASISCRSSKSLLYKDGKTYLNWLQQRP
GQPPRLLIYLMSTRASGVPDRFSGSGAGTDFTLKISRVEAEDVGV
YYCQQLVDYPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
>BBBB1506|heavy_chain_2|
SEQ ID NO: 22
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLSCAVKGFYPSDIAVEWESNGQPENNYKTTPPVLDSDGSFFLV
SKLTVDKSRWQQGNVFSCSVMHEALHNRFTQKSLSLSPGKBBBB1505
>BBBB1505|heavy_chain_1|
SEQ ID NO: 48
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLWCLVKGFYPSDIAVEWESNGQPENNYKTTPPVLDSDGSFFLY
SKLTVDKSRWQQGNVFSCSVMHEALHNHYTQKSLSLSPGKGGAGG
AQVQLVESGGGVVQPGRSLRLSCAASGFTESSYGMHWVRQAPGKG
LEWVAIISYDGSNKHYADSVKGRFTISRDNSKNTLYLQMNSLRAE
DTAVYYCARAPSSFYFDYWGQGTLVTVSSGTEGKSSGSGSESKST
DIVMTQSPDSLAVSLGERATINCKSSQSVLFSSNNKNYLAWYQQK
PGQPPKLLIYWASTRESGVPDRESGSGSGTDFTLTISSLQAEDVA
VYYCQQYYSTPPTFGQGTKVVIK
>BBBB1505|light_chain_1/2|
SEQ ID NO: 60
DIVMTQTPLSSPVTLGQPASISCRSSKSLLYKDGKTYLNWFQQRP
GQSPRLLIYLMSTRASGVPDRFSGSGAGTDFTLKISRVEAEDVGV
YYCQQLVDYPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
>BBBB1505|heavy_chain_2|
SEQ ID NO: 22
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLSCAVKGFYPSDIAVEWESNGQPENNYKTTPPVLDSDGSFFLV
SKLTVDKSRWQQGNVFSCSVMHEALHNRFTQKSLSLSPGK
BBBB1503
>BBBB1503|heavy_chain_1|
SEQ ID NO: 49
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLWCLVKGFYPSDIAVEWESNGQPENNYKTTPPVLDSDGSFFLY
SKLTVDKSRWQQGNVFSCSVMHEALHNHYTQKSLSLSPGKGGAGG
AQVQLVESGGGVVQPGRSLRLSCAASGFTFSSYGMHWVRQAPGKG
LEWVAIISYDGSNKHYADSVKGRFTISRDNSKNTLYLQMNSLRAE
DTAVYYCARAPSHFYFDYWGQGTLVTVSSGTEGKSSGSGSESKST
DIVMTQSPDSLAVSLGERATINCKSSQSVLESSNNKNYLAWYQQK
PGQPPKLLIYWASTRESGVPDRESGSGSGTDFTLTISSLQAEDVA
VYYCQQYYSTPPTFGQGTKVVIK
>BBBB1503|light_chain_1/2|
SEQ ID NO: 60
DIVMTQTPLSSPVTLGQPASISCRSSKSLLYKDGKTYLNWFQQRP
GQSPRLLIYLMSTRASGVPDRFSGSGAGTDFTLKISRVEAEDVGV
YYCQQLVDYPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
>BBBB1503|heavy_chain_2|
SEQ ID NO: 22
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
`
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLSCAVKGFYPSDIAVEWESNGQPENNYKTTPPVLDSDGSFFLV
SKLTVDKSRWQQGNVFSCSVMHEALHNRFTQKSLSLSPGK
>PT1B1183 VH
SEQ ID NO: 18
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSS
>PT1B1183 VL
SEQ ID NO: 59
DIVMTQTPLSSPVTLGQPASISCRSSKSLLYKDGKTYLNWFQQRP
GQSPRLLIYLMSTRASGVPDRFSGSGAGTDFTLKISRVEAEDVGV
YYCQQLVDYPLTFGQGTKLEIK
>PT1B1183 HCDR1
SEQ ID NO: 4
GYTFTSYWIT
>PT1B1183 HCDR2
SEQ ID NO: 5
DIHPGRGSTK
>PT1B1183 HCDR3
SEQ ID NO: 6
RWGFDY
>PT1B1183 LCDR1
SEQ ID NO: 7
RSSKSLLYKDGKTYLN
>PT1B1183 LCDR2
SEQ ID NO: 8
LMSTRAS
>PT1B1183 LCDR3
SEQ ID NO: 9
QQLVDYPLT
>PT1B1183 HC1
SEQ ID NO: 20
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLWCLVKGFYPSDIAVEWESNGQPENNYKTTPPVLDSDGSFFLY
SKLTVDKSRWQQGNVFSCSVMHEALHNHYTQKSLSLSPGK
>PT1B1183 LC
SEQ ID NO: 60
DIVMTQTPLSSPVTLGQPASISCRSSKSLLYKDGKTYLNWFQQRP
GQSPRLLIYLMSTRASGVPDRFSGSGAGTDFTLKISRVEAEDVGV
YYCQQLVDYPLTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
>PT1B1183 HC2
SEQ ID NO: 22
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPEAAGGPSVFLFPPKPKDTLYITREPEVTCVVVSVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLSCAVKGFYPSDIAVEWESNGQPENNYKTTPPVLDSDGSFFLV
SKLTVDKSRWQQGNVFSCSVMHEALHNRFTQKSLSLSPGK
>PT1B1183 HC
SEQ ID NO: 62
QVQLVQSGAEVKKPGASVKVSCKASGYTFTSYWITWVRQRPGQGL
EWMGDIHPGRGSTKYAQKLQGRVTMTTDTSTSTAYMELRSLRSDD
TAVYYCARRWGFDYWGQGTLVTVSSASTKGPSVFPLAPSSKSTSG
GTAALGCLVKDYFPEPVTVSWNSGALTSGVHTFPAVLQSSGLYSL
SSVVTVPSSSLGTQTYICNVNHKPSNTKVDKKVEPKSCDKTHTCP
PCPAPELLGGPSVFLFPPKPKDTLMISRTPEVTCVVVDVSHEDPE
VKFNWYVDGVEVHNAKTKPREEQYNSTYRVVSVLTVLHQDWLNGK
EYKCKVSNKALPAPIEKTISKAKGQPREPQVYTLPPSREEMTKNQ
VSLTCLVKGFYPSDIAVEWESNGQPENNYKTTPPVLDSDGSFFLY
SKLTVDKSRWQQGNVFSCSVMHEALHNHYTQKSLSLSPGK

1. An isolated humanized antibody or antigen-binding fragment thereof that binds to a tau protein at an epitope of the tau protein consisting of or within the amino acid sequence of SEQ ID NO: 1, wherein the antibody or antigen-binding fragment thereof binds paired helical filament (PHF)-tau, preferably human PHF-tau, wherein the epitope of the tau protein comprises one or more of phosphorylated T427, phosphorylated S433 and phosphorylated S435 of the tau protein, but does not comprise all of phosphorylated T427, phosphorylated S433 and phosphorylated S435.

2. The isolated humanized antibody or antigen-binding fragment thereof of claim 1 comprising immunoglobulin heavy chain complementarity determining regions (HCDRs) HCDR1, HCDR2 and HCDR3 having the polypeptide sequences of SEQ ID NOs: 4, 5 and 6, respectively; and immunoglobulin light chain complementarity determining regions (LCDRs) LCDR1, LCDR2 and LCDR3 having the polypeptide sequences of SEQ ID NOs: 7 or 14, 8 and 9, respectively; wherein the isolated humanized antibody or antigen-binding fragment thereof comprises a heavy chain variable region having a polypeptide sequence at least 90% identical to SEQ ID NO: 12 or 18, or a light chain variable region having a polypeptide sequence at least 90% identical to SEQ ID NO: 13, 19, 23 or 59.

3. The isolated humanized antibody or antigen-binding fragment thereof of claim 2, comprising a heavy chain variable region having the polypeptide sequence of SEQ ID NO: 12 or 18, and a light chain variable region having the polypeptide sequence of SEQ ID NO: 13, 19, 23 or 59.

4. The isolated humanized antibody or antigen-binding fragment thereof of claim 1, comprising:
(a) a heavy chain variable region having the polypeptide sequence of SEQ ID NO: 12, and a light chain variable region having the polypeptide sequence of SEQ ID NO: 13;
(b) a heavy chain variable region having the polypeptide sequence of SEQ ID NO: 18, and a light chain variable region having the polypeptide sequence of SEQ ID NO: 19;
(c) a heavy chain variable region having the polypeptide sequence of SEQ ID NO: 12, and a light chain variable region having the polypeptide sequence of SEQ ID NO: 23; or
(d) a heavy chain variable region having the polypeptide sequence of SEQ ID NO: 18, and a light chain variable region having the polypeptide sequence of SEQ ID NO: 59.

5. The isolated humanized antibody or antigen-binding fragment thereof of claim 1, comprising:
(a) a first heavy chain having the polypeptide sequence of SEQ ID NOs: 15 or 20;
(b) two light chains each independently having the polypeptide sequence of SEQ ID NOs: 16, 21, 24 or 60; and
(c) a second heavy chain having the polypeptide sequence of SEQ ID NO: 17 or 22.

6. The isolated humanized antibody or antigen-binding fragment thereof of claim 1, comprising:
(a) a heavy chain having the polypeptide sequence of SEQ ID NO: 61 or 62; and
(b) a light chain having the polypeptide sequence of SEQ ID NO: 16 or 24, or 21 or 60, respectively.

7. A conjugate comprising the isolated humanized antibody or antigen-binding fragment thereof of claim 1 coupled to an anti-CD98 or anti-TfR antibody or antigen-binding fragment thereof.

8. The conjugate of claim 7, wherein the anti-CD98 or anti-TfR antibody or antigen-binding fragment thereof binds to a CD98, preferably human CD98hc, or a TfR, preferably human TfR1, respectively, with a dissociation constant K_{D} of at least 1 nM, preferably 1-500 nM, at neutral pH and an off-rate constant k_{d} of at least 10⁻⁴ sec⁻¹, preferably 10⁻⁴ to 10⁻¹ sec⁻¹, at an acidic pH, preferably pH 5.

9. The conjugate of claim 7, wherein the anti-CD98 or anti-TfR antibody or antigen-binding fragment thereof has an off-rate constant k_{d} of 2×10⁻² to 2×10⁻⁴ sec⁻¹, preferably 8×10⁻³ sec⁻¹ at the neutral pH.

10. The conjugate of claim 7, wherein the anti-CD98 or anti-TfR antibody or antigen-binding fragment thereof comprises a heavy chain variable region comprising HCDR1, HCDR2 and HCDR3, and a light chain variable region comprising LCDR1, LCDR2 and LCDR3, wherein:
(a) the HCDR1, HCDR2, HCDR3, LCDR1, LCDR2 and LCDR3 of the anti-CD98 antibody or antigen-binding fragment thereof have the amino acid sequences of SEQ ID NOs: 26, 27, 28 or 33, 29, 30 and 31, respectively; or
(b) the HCDR1, HCDR2, HCDR3, LCDR1, LCDR2 and LCDR3 of the anti-TfR antibody or antigen-binding fragment thereof have the amino acid sequences of SEQ ID NOs: 35, 36, 37, 38, 39 and 40, respectively.

11. The conjugate of claim 7, wherein the anti-CD98 or anti-TfR antibody or antigen-binding fragment thereof is a single-chain variable fragment (scFv) comprising the heavy chain variable region covalently linked to the light chain variable region via a linker, preferably, the linker has the amino acid sequence of SEQ ID NO: 55 or 56, more preferably, the scFv comprises an amino acid sequence having at least 80%, such as at least 85%, 90%, 95% or 100%, sequence identity to the amino acid sequence of SEQ ID NO: 25, SEQ ID NO: 32, SEQ ID NO: 34, or SEQ ID NO: 41.

12. A fusion construct comprising the conjugate of claim 7, wherein the anti-CD98 or anti-TfR antibody or antigen-binding fragment thereof is covalently linked to the carboxy terminus of only one of the two heavy chains of the isolated humanized antibody or antigen-binding fragment thereof via a linker, preferably wherein the linker has the amino acid sequence of SEQ ID NO: 52.

13. The fusion construct of claim 12, wherein each of the two heavy chains of the isolated humanized antibody or antigen binding fragment thereof comprises one or more heterodimeric mutations, such as a modified heterodimeric CH3 domain, or one or more knob and hole mutations, as compared to a wild-type CH3 domain polypeptide.

14. The fusion construct of claim 13, wherein the modified heterodimeric CH3 domain of the first heavy chain comprises amino acid modifications at positions T350, L351, F405, and Y407, and the modified heterodimeric CH3 domain of the second heavy chain comprises amino acid modifications at positions T350, T366, K392 and T394, wherein the amino acid modification at position T350 is T350V, T350I, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V or T366M, the amino acid modification at position K392 is K392F, K392L or K392M, and the amino acid modification at position T394 is T394W, and wherein the numbering of amino acid residues is according to the EU index as set forth in Kabat.

15. The fusion construct of claim 13, wherein the modified heterodimeric CH3 domain of the first heavy chain comprises mutation T366W, and the modified heterodimeric CH3 domain of the second heavy chain comprises mutations T366S, L368A and Y407V.

16. The fusion construct of claim 12, wherein the isolated humanized antibody or antigen-binding fragment thereof comprises one or more mutations in the Fc domain that enhance binding of the fusion to the neonatal Fc receptor (RcRn), preferably the one or more mutations enhance the binding at an acidic pH, more preferably the Fc has the M252Y/S254T/T256E (YTE) mutations, wherein the numbering of amino acid residues is according to the EU index as set forth in Kabat.

17. The fusion construct of claim 12, wherein the isolated humanized antibody or antigen binding fragment thereof comprises one or more mutations in the Fc domain that reduce or eliminate the effector function, preferably the Fc has one or more amino acid modifications at positions L234, L235, D270, N297, E318, K320, K322, P331, and P329, such as one, two or three mutations of L234A, L235A and P331S, wherein the numbering of amino acid residues is according to the EU index as set forth in Kabat.

18. A fusion construct comprising:
(a) a first heavy chain having an amino acid sequence selected from the group consisting of SEQ ID NOs: 42-49;
(b) two light chains each independently having an amino acid sequence selected from the group consisting of SEQ ID NOs: 16, 21, 24 and 60; and
(c) a second heavy chain having an amino acid sequence selected from the group consisting of SEQ ID NOs: 17 and 22.

19. An isolated nucleic acid encoding the isolated humanized antibody or antigen-binding fragment thereof of claim 1.

20. A vector comprising the isolated nucleic acid of claim 19.

21-33. (canceled)


SEQUENCE LISTING (62 sequences)

SEQ ID NO: 1 - protein, 17 aa, Homo sapiens
SPQLATLADEVSASLAK

SEQ ID NO: 2 - protein, 115 aa, Artificial Sequence
  PT1B545 VH
QVQLQQPGAELVKPGASVKMSCKASGYTFTSYWITWVKQRPGQGLEWIGDIHPGRGSTKSNEKFKSKATLTVDTSSSTAYMQFSSLTSEDSAVYYCARRWGFDYWGQGTTLTVSS

SEQ ID NO: 3 - protein, 112 aa, Artificial Sequence
  PT1B545 VL
DIVITQDELSNPVTSGESVSISCRSSKSLLYKDGKTYLNWFLQRPGQSPQLLIYLMSTRASGVSDRFSGSGSGTDFTLEISRVKAEDVGVYYCQQLVDYPLTFGAGTKLELK

SEQ ID NO: 4 - protein, 10 aa, Artificial Sequence
  PT1B545 HCDR1
GYTFTSYWIT

SEQ ID NO: 5 - protein, 10 aa, Artificial Sequence
  PT1B545 HCDR2
DIHPGRGSTK

SEQ ID NO: 6 - protein, 6 aa, Artificial Sequence
  PT1B545 HCDR3
RWGFDY

SEQ ID NO: 7 - protein, 16 aa, Artificial Sequence
  PT1B545 LCDR1
RSSKSLLYKDGKTYLN

SEQ ID NO: 8 - protein, 7 aa, Artificial Sequence
  PT1B545 LCDR2
LMSTRAS

SEQ ID NO: 9 - protein, 9 aa, Artificial Sequence
  PT1B545 LCDR3
QQLVDYPLT

SEQ ID NO: 10 - protein, 445 aa, Artificial Sequence
  PT1B545 HC
QVQLQQPGAELVKPGASVKMSCKASGYTFTSYWITWVKQRPGQGLEWIGDIHPGRGSTKSNEKFKSKATLTVDTSSSTAYMQFSSLTSEDSAVYYCARRWGFDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 11 - protein, 219 aa, Artificial Sequence
  PT1B545 LC
DIVITQDELSNPVTSGESVSISCRSSKSLLYKDGKTYLNWFLQRPGQSPQLLIYLMSTRASGVSDRFSGSGSGTDFTLEISRVKAEDVGVYYCQQLVDYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - protein, 115 aa, Artificial Sequence
  PT1B901 VH
QVQLVQSGAEVKKPGASVKVSCKASGYTFTSYWITWVRQAPGQGLEWMGDIHPGRGSTKYAQKLQGRVTLTTDTSTSTAYMELRSLRSDDTAVYYCARRWGFDYWGQGTLVTVSS

SEQ ID NO: 13 - protein, 112 aa, Artificial Sequence
  PT1B901 VL
DIVITQTPLSLPVTPGEPASISCRSSKSLLYKDLKTYLNWFLQKPGQSPQLLIYLMSTRASGVPDRFSGSGSGTDFTLKISRVEAEDVGVYYCQQLVDYPLTFGQGTKLEIK

SEQ ID NO: 14 - protein, 16 aa, Artificial Sequence
  PT1B901 LCDR1
RSSKSLLYKDLKTYLN

SEQ ID NO: 15 - protein, 445 aa, Artificial Sequence
  PT1B901 HC1
QVQLVQSGAEVKKPGASVKVSCKASGYTFTSYWITWVRQAPGQGLEWMGDIHPGRGSTKYAQKLQGRVTLTTDTSTSTAYMELRSLRSDDTAVYYCARRWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 219 aa, Artificial Sequence
  PT1B901 LC1/2
DIVITQTPLSLPVTPGEPASISCRSSKSLLYKDLKTYLNWFLQKPGQSPQLLIYLMSTRASGVPDRFSGSGSGTDFTLKISRVEAEDVGVYYCQQLVDYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 445 aa, Artificial Sequence
  PT1B901 HC2
QVQLVQSGAEVKKPGASVKVSCKASGYTFTSYWITWVRQAPGQGLEWMGDIHPGRGSTKYAQKLQGRVTLTTDTSTSTAYMELRSLRSDDTAVYYCARRWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 18 - protein, 115 aa, Artificial Sequence
  PT1B1153 VH
QVQLVQSGAEVKKPGASVKVSCKASGYTFTSYWITWVRQRPGQGLEWMGDIHPGRGSTKYAQKLQGRVTMTTDTSTSTAYMELRSLRSDDTAVYYCARRWGFDYWGQGTLVTVSS

SEQ ID NO: 19 - protein, 112 aa, Artificial Sequence
  PT1B1153 VL
DIVMTQTPLSSPVTLGQPASISCRSSKSLLYKDGKTYLNWLQQRPGQPPRLLIYLMSTRASGVPDRFSGSGAGTDFTLKISRVEAEDVGVYYCQQLVDYPLTFGQGTKLEIK

SEQ ID NO: 20 - protein, 445 aa, Artificial Sequence
  PT1B1153 HC1
QVQLVQSGAEVKKPGASVKVSCKASGYTFTSYWITWVRQRPGQGLEWMGDIHPGRGSTKYAQKLQGRVTMTTDTSTSTAYMELRSLRSDDTAVYYCARRWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 219 aa, Artificial Sequence
  PT1B1153 LC1/2
DIVMTQTPLSSPVTLGQPASISCRSSKSLLYKDGKTYLNWLQQRPGQPPRLLIYLMSTRASGVPDRFSGSGAGTDFTLKISRVEAEDVGVYYCQQLVDYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 445 aa, Artificial Sequence
  PT1B1153 HC2
QVQLVQSGAEVKKPGASVKVSCKASGYTFTSYWITWVRQRPGQGLEWMGDIHPGRGSTKYAQKLQGRVTMTTDTSTSTAYMELRSLRSDDTAVYYCARRWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 23 - protein, 112 aa, Artificial Sequence
  PT1B635 VL
DIVITQTPLSLPVTPGEPASISCRSSKSLLYKDGKTYLNWFLQKPGQSPQLLIYLMSTRASGVPDRFSGSGSGTDFTLKISRVEAEDVGVYYCQQLVDYPLTFGQGTKLEIK

SEQ ID NO: 24 - protein, 219 aa, Artificial Sequence
  PT1B635 LC1/2
DIVITQTPLSLPVTPGEPASISCRSSKSLLYKDGKTYLNWFLQKPGQSPQLLIYLMSTRASGVPDRFSGSGSGTDFTLKISRVEAEDVGVYYCQQLVDYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 247 aa, Artificial Sequence
  C98B72 scFv
QVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSSFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIK

SEQ ID NO: 26 - protein, 10 aa, Artificial Sequence
  C98B72 HCDR1
GFTFSSYGMH

SEQ ID NO: 27 - protein, 10 aa, Artificial Sequence
  C98B72 HCDR2
IISYDGSNKH

SEQ ID NO: 28 - protein, 9 aa, Artificial Sequence
  C98B72 HCDR3
APSSFYFDY

SEQ ID NO: 29 - protein, 17 aa, Artificial Sequence
  C98B72 LCDR1
KSSQSVLFSSNNKNYLA

SEQ ID NO: 30 - protein, 7 aa, Artificial Sequence
  C98B72 LCDR2
WASTRES

SEQ ID NO: 31 - protein, 9 aa, Artificial Sequence
  C98B72 LCDR3
QQYYSTPPT

SEQ ID NO: 32 - protein, 247 aa, Artificial Sequence
  C98B73 scFv
QVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSHFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIK

SEQ ID NO: 33 - protein, 9 aa, Artificial Sequence
  C98B73 HCDR3
APSHFYFDY

SEQ ID NO: 34 - protein, 246 aa, Artificial Sequence
  TFRB321 scFv
EVQLLESGGGLVQPGGSLRLSCADSGFTFSSYAMNWVRQAPGCGLEWVSGISGSGGHTYYADSVKGRFTVSRDNSKNTLYLQMNSLRAEDTAVYYCAREGYDSSGYNPFDYWGQGTQVTVSSGGGSGGSGGCPPCGGSGGSYELTQPPSVSVSPGQTASITCSGDKLGDKYASWYQQKPGQSPVLVIYQDSKRPSGIPERFSGSNSGNTATLTISGTQAMDEADYYCQAWDSSTVVFGCGTKLTVL

SEQ ID NO: 35 - protein, 10 aa, Artificial Sequence
  TFRB321 HCDR1
GFTFSSYAMN

SEQ ID NO: 36 - protein, 10 aa, Artificial Sequence
  TFRB321 HCDR2
GISGSGGHTY

SEQ ID NO: 37 - protein, 13 aa, Artificial Sequence
  TFRB321 HCDR3
EGYDSSGYNPFDY

SEQ ID NO: 38 - protein, 11 aa, Artificial Sequence
  TFRB321 LCDR1
SGDKLGDKYAS

SEQ ID NO: 39 - protein, 7 aa, Artificial Sequence
  TFRB321 LCDR2
QDSKRPS

SEQ ID NO: 40 - protein, 9 aa, Artificial Sequence
  TFRB321 LCDR3
QAWDSSTVV

SEQ ID NO: 41 - protein, 244 aa, Artificial Sequence
  TFRB320 scFv
EVQLLESGGGLVQPGGSLRLSCADSGFTFSSYAMNWVRQAPGKGLEWVSGISGSGGHTYYADSVKGRFTVSRDNSKNTLYLQMNSLRAEDTAVYYCAREGYDSSGYNPFDYWGQGTQVTVSSGTEGKSSGSGSESKSTSYELTQPPSVSVSPGQTASITCSGDKLGDKYASWYQQKPGQSPVLVIYQDSKRPSGIPERFSGSNSGNTATLTISGTQAMDEADYYCQAWDSSTVVFGGGTKLTVL

SEQ ID NO: 42 - protein, 697 aa, Artificial Sequence
  BBBB1626 HC1
QVQLVQSGAEVKKPGASVKVSCKASGYTFTSYWITWVRQAPGQGLEWMGDIHPGRGSTKYAQKLQGRVTLTTDTSTSTAYMELRSLRSDDTAVYYCARRWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGAGGAEVQLLESGGGLVQPGGSLRLSCADSGFTFSSYAMNWVRQAPGCGLEWVSGISGSGGHTYYADSVKGRFTVSRDNSKNTLYLQMNSLRAEDTAVYYCAREGYDSSGYNPFDYWGQGTQVTVSSGGGSGGSGGCPPCGGSGGSYELTQPPSVSVSPGQTASITCSGDKLGDKYASWYQQKPGQSPVLVIYQDSKRPSGIPERFSGSNSGNTATLTISGTQAMDEADYYCQAWDSSTVVFGCGTKLTVL

SEQ ID NO: 43 - protein, 697 aa, Artificial Sequence
  BBBB1624 HC1
QVQLVQSGAEVKKPGASVKVSCKASGYTFTSYWITWVRQRPGQGLEWMGDIHPGRGSTKYAQKLQGRVTMTTDTSTSTAYMELRSLRSDDTAVYYCARRWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGAGGAEVQLLESGGGLVQPGGSLRLSCADSGFTFSSYAMNWVRQAPGCGLEWVSGISGSGGHTYYADSVKGRFTVSRDNSKNTLYLQMNSLRAEDTAVYYCAREGYDSSGYNPFDYWGQGTQVTVSSGGGSGGSGGCPPCGGSGGSYELTQPPSVSVSPGQTASITCSGDKLGDKYASWYQQKPGQSPVLVIYQDSKRPSGIPERFSGSNSGNTATLTISGTQAMDEADYYCQAWDSSTVVFGCGTKLTVL

SEQ ID NO: 44 - protein, 695 aa, Artificial Sequence
  BBBB1622 HC1
QVQLVQSGAEVKKPGASVKVSCKASGYTFTSYWITWVRQAPGQGLEWMGDIHPGRGSTKYAQKLQGRVTLTTDTSTSTAYMELRSLRSDDTAVYYCARRWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGAGGAEVQLLESGGGLVQPGGSLRLSCADSGFTFSSYAMNWVRQAPGKGLEWVSGISGSGGHTYYADSVKGRFTVSRDNSKNTLYLQMNSLRAEDTAVYYCAREGYDSSGYNPFDYWGQGTQVTVSSGTEGKSSGSGSESKSTSYELTQPPSVSVSPGQTASITCSGDKLGDKYASWYQQKPGQSPVLVIYQDSKRPSGIPERFSGSNSGNTATLTISGTQAMDEADYYCQAWDSSTVVFGGGTKLTVL

SEQ ID NO: 45 - protein, 695 aa, Artificial Sequence
  BBBB1620 HC1
QVQLVQSGAEVKKPGASVKVSCKASGYTFTSYWITWVRQRPGQGLEWMGDIHPGRGSTKYAQKLQGRVTMTTDTSTSTAYMELRSLRSDDTAVYYCARRWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGAGGAEVQLLESGGGLVQPGGSLRLSCADSGFTFSSYAMNWVRQAPGKGLEWVSGISGSGGHTYYADSVKGRFTVSRDNSKNTLYLQMNSLRAEDTAVYYCAREGYDSSGYNPFDYWGQGTQVTVSSGTEGKSSGSGSESKSTSYELTQPPSVSVSPGQTASITCSGDKLGDKYASWYQQKPGQSPVLVIYQDSKRPSGIPERFSGSNSGNTATLTISGTQAMDEADYYCQAWDSSTVVFGGGTKLTVL

SEQ ID NO: 46 - protein, 698 aa, Artificial Sequence
  BBBB1548 HC1
QVQLVQSGAEVKKPGASVKVSCKASGYTFTSYWITWVRQAPGQGLEWMGDIHPGRGSTKYAQKLQGRVTLTTDTSTSTAYMELRSLRSDDTAVYYCARRWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGAGGAQVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSSFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIK

SEQ ID NO: 47 - protein, 698 aa, Artificial Sequence
  BBBB1546 HC1
QVQLVQSGAEVKKPGASVKVSCKASGYTFTSYWITWVRQAPGQGLEWMGDIHPGRGSTKYAQKLQGRVTLTTDTSTSTAYMELRSLRSDDTAVYYCARRWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGAGGAQVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSHFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIK

SEQ ID NO: 48 - protein, 698 aa, Artificial Sequence
  BBBB1508 HC1
QVQLVQSGAEVKKPGASVKVSCKASGYTFTSYWITWVRQRPGQGLEWMGDIHPGRGSTKYAQKLQGRVTMTTDTSTSTAYMELRSLRSDDTAVYYCARRWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGAGGAQVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSSFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIK

SEQ ID NO: 49 - protein, 698 aa, Artificial Sequence
  BBBB1506 HC1
QVQLVQSGAEVKKPGASVKVSCKASGYTFTSYWITWVRQRPGQGLEWMGDIHPGRGSTKYAQKLQGRVTMTTDTSTSTAYMELRSLRSDDTAVYYCARRWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGAGGAQVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSHFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIK

SEQ ID NO: 50 - protein, 18 aa, Artificial Sequence
  Linker
GGGSGGSGGCPPCGGSGG

SEQ ID NO: 51 - protein, 16 aa, Artificial Sequence
  Linker
GTEGKSSGSGSESKST

SEQ ID NO: 52 - protein, 6 aa, Artificial Sequence
  Linker
GGAGGA

SEQ ID NO: 53 - protein, 352 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 54 - protein, 381 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 55 - protein, 410 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 56 - protein, 383 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 57 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 58 - protein, 441 aa, Homo sapiens; identical to SEQ ID NO: 57

SEQ ID NO: 59 - protein, 112 aa, Artificial Sequence
  PT1B1183 VL
DIVMTQTPLSSPVTLGQPASISCRSSKSLLYKDGKTYLNWFQQRPGQSPRLLIYLMSTRASGVPDRFSGSGAGTDFTLKISRVEAEDVGVYYCQQLVDYPLTFGQGTKLEIK

SEQ ID NO: 60 - protein, 219 aa, Artificial Sequence
  PT1B1183 LC
DIVMTQTPLSSPVTLGQPASISCRSSKSLLYKDGKTYLNWFQQRPGQSPRLLIYLMSTRASGVPDRFSGSGAGTDFTLKISRVEAEDVGVYYCQQLVDYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 445 aa, Artificial Sequence
  PR1B901 & PT1B635 HC
QVQLVQSGAEVKKPGASVKVSCKASGYTFTSYWITWVRQAPGQGLEWMGDIHPGRGSTKYAQKLQGRVTLTTDTSTSTAYMELRSLRSDDTAVYYCARRWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 445 aa, Artificial Sequence
  PT1B1153 & PT1B1183 HC
QVQLVQSGAEVKKPGASVKVSCKASGYTFTSYWITWVRQRPGQGLEWMGDIHPGRGSTKYAQKLQGRVTMTTDTSTSTAYMELRSLRSDDTAVYYCARRWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK